(19) United States
(12) Patent Application Publication
(10) Pub. No.: US 2012/0082977 A1
(43) Pub. Date: Apr. 5, 2012

(54) ARTICLES WITH MATRIX COMPRISING A CELL EXTRACTANT AND BIODETECTION METHODS THEREOF

(76) Inventors: Raj Rajagopal, Woodbury, MN (US); Robin E. Wright, Inver Grove Heights, MN (US); Caroline M. Ylitalo, Stillwater, MN (US); Smarajit Mitra, West St. Paul, MN (US); Matthew D. Reier, Beaveton, OR (US)

(21) Appl. No.: 13/266,496

(22) PCT Filed: May 6, 2010
(86) PCT No.: PCT/US10/33803

§ 371 Date: Dec. 21, 2011

Related U.S. Application Data
(60) Provisional application No. 61/175,980, filed on May 6, 2009.

(51) Int. Cl. C12Q 1/68; G01N 33/566; C12Q 1/06; C12Q 1/66; C12M 1/34; C12Q 1/04

(52) U.S. Cl. 435/6.1; 435/287.1; 435/287.2; 435/288.7; 435/287.6; 435/34; 435/39; 435/8; 435/7.1

(57) ABSTRACT

Articles are provided for the detection of cells in a sample. The articles include a release element. The release element comprises an encapsulating agent and a cell extractant. The release element controls the release of the cell extractant into a liquid mixture containing the sample. Methods of use are also disclosed.

### CROSS REFERENCE TO RELATED APPLICATION

This application claims the benefit of U.S. Provisional Patent Application No. 61/175,980, filed May 6, 2009, which is incorporated herein by reference in its entirety.

### BACKGROUND

Various tests are available that can be used to assess the presence of biological analytes in a sample (e.g. surface, water, air, etc). Such tests include those based on the detection of ATP using the firefly luciferase reaction (see, for example, U.S. Pat. Nos. 3,971,703 and 4,144,134 and PCT International Publication No. WO2007/061293, each of which is incorporated herein by reference in its entirety), tests based on the detection of protein using colorimetry, tests based on the detection of microorganisms using microbiological culture techniques, and tests based on detection of microorganisms using immunochemical techniques. Surfaces can be sampled using either a swab device or by direct contact with a culture device such as an agar plate. The sample can be analyzed for the presence of live cells and, in particular, live microorganisms.

Results from these tests are often used to make decisions about the cleanliness of a surface. For example, the test may be used to decide whether food-processing equipment has been cleaned well enough to use for production. Although the above tests are useful in the detection of a contaminated surface, they can require numerous steps to perform the test, they may not be able to distinguish quickly and/or easily the presence of live cells from dead cells and, in some cases, they can require long periods of time (e.g., hours or days) before the results can be determined.

The tests may be used to indicate the presence of live microorganisms. For such tests, a cell extractant is often used to release a biological analyte (e.g., ATP) associated with living cells. The presence of extracellular material (e.g., non-cellular ATP released into the environment from dead or stressed animal cells, plant cells, and/or microorganisms) can create a high “background” level of ATP that can complicate the detection of live cells.

In spite of the availability of a number of methods and devices to detect live cells, there remains a need for a simple, reliable test for detecting live cells and, in particular, live microbial cells.

### SUMMARY

In general, the present disclosure relates to articles and methods for detecting live cells in a sample. The articles and methods make possible the rapid detection (e.g., through fluorescence, chemiluminescence, or a color reaction) of the presence of cells such as bacteria on a surface. In some embodiments, the inventive articles are “sample-ready”, i.e., the articles contain all of the necessary features to detect living cells in a sample. The methods feature the use of a cell extractant to facilitate the release of biological analytes from biological cells. The inventive articles and methods include a release element, which controls the release of an effective amount of cell extractant into a liquid mixture comprising a sample. In some aspects, the inventive articles and methods provide a means to distinguish a biological analyte, such as ATP or an enzyme, that is associated with eukaryotic cells (e.g., plant or animal cells) from a similar or identical biological analyte associated with prokaryotic cells (e.g., bacterial cells). Furthermore, the inventive articles and methods provide a means to distinguish a biological analyte that is free in the environment (i.e., an acellular biological analyte) from a similar or identical biological analyte associated with a living cell.

Thus, in one aspect, the present disclosure provides an article for detecting cells in a sample. The article can comprise a housing with an opening configured to receive a sample acquisition device, a sample acquisition device, and a release element comprising a cell extractant. In some embodiments, the release element can be disposed in the housing. In some embodiments, the release element can be disposed on the sample acquisition device. In some embodiments, the sample acquisition device can further comprise a reagent chamber.

In another aspect, the present disclosure provides an article for detecting cells in a sample. The article can comprise a housing with an opening configured to receive a sample, a sample acquisition device comprising a reagent chamber, a cell extractant, and a release element comprising the cell extractant. The release element can be disposed in the reagent chamber.

In any one of the above embodiments, the article can further comprise a frangible barrier that forms a compartment in the housing. In some embodiments, the frangible barrier can comprise the release element comprising the cell extractant. In some embodiments, the compartment can comprise the release element.

In another aspect, the present disclosure provides an article for detecting cells in a sample. The article can comprise a housing with an opening configured to receive a sample, a release element comprising a cell extractant; a delivery element comprising a detection reagent. In some embodiments, the release element and the delivery element are disposed in the housing.

In any one of the above embodiments, the housing can further comprise a compartment. In any one of the above embodiments, the compartment can further comprise a detection reagent.

In any one of the above embodiments, the detection reagent is selected from the group consisting of an enzyme, an enzyme substrate, an indicator dye, a stain, an antibody, and a polynucleotide.

In another aspect, the present disclosure provides a sample acquisition device with a release element disposed thereon. The release element can comprise a cell extractant. In some embodiments, the cell extractant can comprise a microbial cell extractant. In some embodiments, the cell extractant can comprise a somatic cell extractant.

In another aspect, the present disclosure provides a kit. The kit can comprise a housing with an opening configured to receive a sample, a release element comprising a cell extractant, and a detection system. Optionally, the kit can further comprise a sample acquisition device and the opening in the housing can be configured to receive the sample acquisition device. In some embodiments, the detection system can further comprise a delivery element comprising a detection reagent. In some embodiments, the detection reagent can be selected from the group consisting of an enzyme, an enzyme substrate, an indicator dye, a stain, an antibody, and a polynucleotide.

In another aspect, the present disclosure provides a method of detecting cells in a sample. The method can comprise providing a release element comprising a cell extractant, and a sample suspected of containing cells. The method further can comprise forming a liquid mixture comprising the sample and the release element. The method further can comprise detecting an analyte in the liquid mixture.

In another aspect, the present disclosure provides a method of detecting cells in a sample. The method can comprise providing a sample acquisition device and a housing. The housing can include an opening configured to receive the sample acquisition device and a release element comprising the cell extractant. The release element can be disposed in the housing. The method further can comprise obtaining sample material with the sample acquisition device, forming a liquid mixture comprising the sample material and the release element, and detecting an analyte in the liquid mixture.

In any one of the above embodiments, the release element can comprise an encapsulating agent. In any one of the above embodiments, the release element can comprise a matrix. The matrix can comprise a pre-formed matrix, a formed matrix, or an admixture comprising an excipient. In any one of the above embodiments, the cell extractant is selected from the group consisting of a quaternary amine, a biguanide, a nonionic surfactant, a cationic surfactant, a phenolic, a cytolytic peptide, and an enzyme.

In any one of the above embodiments, the method further can comprise detecting the analyte using a detection system. In any one of the above embodiments, the method further can comprise quantifying an amount of the analyte. In any one of the above embodiments, the method further can comprise quantifying an amount of the analyte two or more times. In any one of the above embodiments, the method further can comprise releasing the cell extractant from the release element using a release factor.

### GLOSSARY

“Biological analytes”, as used herein, refers to molecules, or derivatives thereof, that occur in or are formed by an organism. For example, a biological analyte can include, but is not limited to, at least one of an amino acid, a nucleic acid, a polypeptide, a protein, a polynucleotide, a lipid, a phospholipid, a saccharide, a polysaccharide, and combinations thereof. Specific examples of biological analytes can include, but are not limited to, a metabolite (e.g., staphylococcal enterotoxin), an allergen (e.g., peanut allergen(s), a hormone, a toxin (e.g., Bacillus diarrheal toxin, aflatoxin, etc.), RNA (e.g., mRNA, total RNA, tRNA, etc.), DNA (e.g., plasmid DNA, plant DNA, etc.), a tagged protein, an antibody, an antigen, and combinations thereof.

“Sample acquisition device” is used herein in the broadest sense and refers to an implement used to collect a liquid, semisolid, or solid sample material. Nonlimiting examples of sample acquisition devices include swabs, wipes, sponges, scoops, spatulas, pipettes, pipette tips, and siphon hoses.

As used herein, “chromonic materials” (or “chromonic compounds”) refers to large, multi-ring molecules typically characterized by the presence of a hydrophobic core surrounded by various hydrophilic groups (see, for example, Attwood, T. K., and Lydon, J. E., Molec. Crystals Liq. Crystals, 108, 349 (1984)). The hydrophobic core can contain aromatic and/or non-aromatic rings. When in solution, these chromonic materials tend to aggregate into a nematic ordering characterized by a long-range order.

As used herein, “release element” refers to a structure that holds a cell extractant. The release element includes physical and/or chemical components selected to limit the diffusion of a cell extractant from a region of relatively high concentration to a region of relatively low concentration.

“Encapsulating agent” refers to a type of release element. An encapsulating agent, as used herein, is a material that substantially surrounds the cell extractant.

As used herein, “matrix” refers to a solid or semisolid material into which cell extractant can be substantially interfused.

As used herein, the term “hydrogel” refers to a polymeric material that is hydrophilic and that is either swollen or capable of being swollen with a polar solvent. The polymeric material typically swells but does not dissolve when contacted with the polar solvent. That is, the hydrogel is insoluble in the polar solvent. The swollen hydrogel can be dried to remove at least some of the polar solvent.

“Cell extractant”, as used herein, refers to any compound or combination of compounds that alters cell membrane or cell wall permeability or disrupts the integrity of (i.e., lyses or causes the formation of pores in) the membrane and/or cell wall of a cell (e.g., a somatic cell or a microbial cell) to effect extraction or release of a biological analyte normally found in living cells.

“Detection system”, as used herein, refers to the components used to detect a biological analyte and includes enzymes, enzyme substrates, binding partners (e.g. antibodies or receptors), labels, dyes, and instruments for detecting light absorbance or reflectance, fluorescence, and/or luminescence (e.g. bioluminescence or chemiluminescence).

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

As used herein, “a,” “an,” “the,” “at least one,” and “one or more” are used interchangeably. Thus, for example, a housing that comprises “a” detection reagent can be interpreted to mean that the housing can include “one or more” detection reagents.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

The invention will be further explained with reference to the drawing figures listed below, where like structure is referenced by like numerals throughout the several views.

FIG. 1 shows a side view of one embodiment of a sample acquisition device with a release element disposed thereon.

FIG. 2 shows a partial cross-section view of one embodiment of a sample acquisition device comprising an enclosure containing a release element.

FIG. 3 shows a cross-section view of one embodiment of a housing with a release element disposed therein.

FIG. 4 shows a cross-section view of the housing of FIG. 3, further comprising a frangible seal.

FIG. 5 shows a cross-section view of one embodiment of a housing containing a release element, a frangible seal, and a detection reagent.

FIG. 6A shows a cross-section view of one embodiment of a detection device comprising the housing of FIG. 5 and side view of a sample acquisition device disposed in a first position therein.

FIG. 6B shows a partial cross-section view of the detection device of FIG. 6A with the sample acquisition device disposed in a second position therein.

FIG. 7 shows a partial cross-section view of one embodiment of a detection device comprising a housing, a plurality of frangible seals with a release element disposed there between, and a sample acquisition device.

FIG. 8 shows a partial cross-section view of one embodiment of a detection device comprising a housing, a carrier comprising a release element, and a sample acquisition device.

FIG. 9 shows a bottom perspective view of the carrier of FIG. 8.

FIG. 10 shows a top perspective view of one embodiment of a release element with a cell extractant dispersed therein.

### DETAILED DESCRIPTION

All patents, patent applications, government publications, government regulations, and literature references cited in this specification are hereby incorporated herein by reference in their entirety. In case of conflict, the present description, including definitions, will control.

Biological analytes can be used to detect the presence of biological material, such as live cells in a sample. Biological analytes can be detected by various reactions (e.g., binding reactions, catalytic reactions, and the like) in which they can participate.

Chemiluminescent reactions can be used in various forms to detect cells, such as bacterial cells, in fluids and in processed materials. In some embodiments of the present disclosure, a chemiluminescent reaction based on the reaction of adenosine triphosphate (ATP) with luciferin in the presence of the enzyme luciferase to produce light provides the chemical basis for the generation of a signal to detect a biological analyte, ATP. Since ATP is present in all living cells, including all microbial cells, this method can provide a rapid assay to obtain a quantitative or semiquantitative estimate of the number of living cells in a sample. Early discourses on the nature of the underlying reaction, the history of its discovery, and its general area of applicability, are provided by E. N. Harvey (1957), A History of Luminescence: From the Earliest Times Until 1900, Amer. Phil. Soc., Philadelphia, Pa.; and W. D. McElroy and B. L. Strehler (1949), Arch. Biochem. Biophys. 22:420-433.

ATP detection is a reliable means to detect bacteria and other microbial species because all such species contain some ATP. Chemical bond energy from ATP is utilized in the bioluminescent reaction that occurs in the tails of the firefly Photinus pyralis. The biochemical components of this reaction can be isolated free of ATP and subsequently used to detect ATP in other sources. The mechanism of this firefly bioluminescence reaction has been well characterized (DeLuca, M., et al., 1979 Anal. Biochem. 95:194-198).

The inventive articles and methods of the present disclosure provide simple means for conveniently controlling the release of biological analytes from living cells in order to determine the presence, optionally the type (e.g., microbial or nonmicrobial), and optionally the quantity of living cells in an unknown sample. The articles and methods include a release element.

### Release Element:

Release elements, according to the present disclosure, include encapsulating materials. Encapsulating materials generally act as a physical barrier and/or a diffusion barrier to prevent the immediate dissolution, for a period of time, of an effective amount of cell extractant into a liquid mixture (for example, an aqueous mixture comprising a sample).

In some embodiments, the encapsulating materials may be activated to release an effective amount of cell extractant after the encapsulant is exposed to an activating stimulus. Activation may include, for example, dissolution or partial dissolution of the encapsulating material, permeabilization (e.g., by swelling a partially dehydrated polymer) of the encapsulating material, disintegration or partial disintegration of the encapsulating material (e.g., by melting a solid material such as, for example a wax).

In some embodiments, encapsulating material can comprise a chromonic material, as disclosed in U.S. Patent Application No. 61/175,996, filed on May 6, 2009 and entitled ARTICLES WITH SHELL STRUCTURES INCLUDING A CELL EXTRACTANT AND BIODETECTION METHODS THEREOF, which is incorporated herein by reference in its entirety.

In some embodiments, the encapsulating material can comprise a matrix. In some embodiments, the matrix comprises a material (e.g., a polymeric material or a nonpolymer material such as a ceramic) that is substantially insoluble in a liquid (for example, an aqueous liquid comprising a sample). In some embodiments, the matrix comprises an excipient that is substantially soluble and/or dispersible at ambient temperature in an aqueous solution. In some embodiments, the matrix comprises an excipient that is substantially insoluble and nondispersible at ambient temperature in an aqueous solution (i.e., the dissolution or dispersion of the excipient can be triggered by a temperature shift and/or the addition of a chemical trigger).

In some embodiments, the matrixes can be pre-formed matrixes (i.e., matrixes that are formed before the matrixes are infused with a cell extractant). In these embodiments, a cell extractant can be loaded into a matrix by placing the matrix into a liquid containing the cell extractant and allowing the cell extractant to diffuse into the matrix material, as described below in Preparative Examples 5 and 6, for example. In some embodiments, matrix precursors can be mixed in a solution with the cell extractant and the matrix is formed with the cell extractant dispersed within the matrix, such as the polymer matrix described below in Preparative Example 1, for example. In another embodiment, the cell extractant can be dispersed in wax, as described, for example, in U.S. Patent Application Publication No. US2005/0152992, which is incorporated herein by reference in its entirety.

Encapsulating materials can comprise a hydrogel. The use of hydrogels in articles and methods for detecting cells in a sample is disclosed in U.S. Patent Application Nos. 61/101,546 (Attorney Docket No. 64686US002) and 61/101,563 (Attorney Docket No. 64806US002), both filed on Sep. 30, 2008 and respectively entitled BIODETECTION ARTICLES and BIODETECTION METHODS, each of which is incorporated herein by reference in its entirety.

Hydrogels broadly include crosslinked hydrogels, swollen hydrogels, and dried or partially-dried hydrogels. Suitable hydrogels of the present disclosure include, for example, the hydrogels, and polymeric beads made there from, described in International Patent Publication No. WO 2007/146722, which is incorporated herein by reference in its entirety.

Other suitable hydrogels include polymers comprising ethylenically unsaturated carboxyl-containing monomers and comonomers selected from carboxylic acids, vinyl sulfonic acid, cellulosic monomer, polyvinyl alcohol, as described in U.S. Patent Application Publication No. US2004/0157971; polymers comprising starch, cellulose, polyvinyl alcohol, polyethylene oxide, polypropylene glycol, and copolymers thereof, as described in U.S. Patent Application Publication No. US 2006/0062854; polymers comprising multifunctional poly(alkylene oxide) free-radically polymerizable macromonomer with molecular weights less than 2000 daltons, as described in U.S. Pat. No. 7,005,143; polymers comprising silane-functionalized polyethylene oxide that cross-link upon exposure to a liquid medium, as described in U.S. Pat. No. 6,967,261; polymers comprising polyurethane prepolymer with at least one alcohol selected from polyethylene glycol, polypropylene glycol, and propylene glycol, as described in U.S. Pat. No. 6,861,067; and polymers comprising a hydrophilic polymer selected from polysaccharide, polyvinylpyrolidone, polyvinyl alcohol, polyvinyl ether, polyurethane, polyacrylate, polyacrylamide, collagen and gelatin, as described in U.S. Pat. No. 6,669,981, the disclosures of which are all herein incorporated by reference in their entirety. Other suitable hydrogels include agar, agarose, polyacrylamide hydrogels, and derivatives thereof.

The present disclosure provides for articles and methods that include a shaped hydrogel. Shaped hydrogels include hydrogels shaped into, for example, beads, sheets, ribbons, and fibers. Additional examples of shaped hydrogels and exemplary processes by which shaped hydrogels can be produced are disclosed in U.S. Patent Application Publication No. 2008/0207794 A1, entitled POLYMERIC FIBERS AND METHODS OF MAKING and U.S. Patent Application No. 61/013,085 (Attorney Docket No. 63498US002), entitled METHODS OF MAKING SHAPED POLYMERIC MATERIALS, both of which are incorporated herein by reference in their entirety.

Hydrogels of the present disclosure can comprise a cell extractant. Hydrogels comprising a cell extractant can be made by two fundamental processes. In a first process, the cell extractant is incorporated into the hydrogel during the synthesis of the hydrogel polymer. Examples of the first process can be found in International Patent Publication No. WO 2007/146722 and in Preparative Example 1 described herein. In a second process the cell extractant is incorporated into the hydrogel after the synthesis of the hydrogel polymer. For example, the hydrogel is placed in a solution of cell extractant and the cell extractant is allowed to absorb into and/or adsorb to the hydrogel. An example of the second process is described in Preparative Example 5 below. A further example of the second process is the incorporation of an ionic monomer into the hydrogel, such as the incorporation of a cationic monomer into the hydrogel, as described herein in Preparative Example 2.

In some applications, it may be desirable that the release element containing a cell extractant is in a dry or partially-dried state. Certain release elements (e.g., water-swollen hydrogels) can be dried, for example, by methods known to those skilled in the art, including evaporative processes, drying in convection ovens, microwave ovens, and vacuum ovens as well as freeze-drying. When the dried or partially-dried release element is exposed to a liquid or aqueous solution, the cell extractant can diffuse from the release element. The cell extractant can remain essentially dormant in the release element until exposed to a liquid or aqueous solution. That is, the cell extractant can be stored within the dry or partially-dried release element until the release element is exposed to a liquid. This can prevent the waste or loss of the cell extractant when not needed and can improve the stability of many moisture sensitive cell extractants that may degrade by hydrolysis, oxidation, or other mechanisms.

In some embodiments, certain release elements (e.g., water-swollen hydrogels) that do not contain a cell extractant can be dried. Optionally, the dried material can be packaged (e.g., in a vacuum package). The dried material subsequently can be rehydrated in a solution comprising a cell extractant, thereby loading the rehydrated hydrogel with the cell extractant. Advantageously, this process allows a hydrogel to be produced and dried at one location and transported in a dry state to a second location, where the dried hydrogel can be loaded with a cell extractant by rehydrating the dried hydrogel in a solution (e.g., an aqueous solution) comprising a cell extractant. Optionally, after rehydrating the hydrogel with the cell-extractant solution, the swollen hydrogel can be dried with the cell extractant therein and/or thereon, as described above.

In some embodiments, the encapsulating materials may be activated to release an effective amount of cell extractant after the encapsulant is exposed to an activating stimulus such as pressure, shear, heat, light, pH change, exposure to another chemical, ionic strength change and the like. Activation may result in, for example, dissolution or partial dissolution of the encapsulating material, permeabilization of the encapsulating material (e.g. disruption of a lipid bilayer), and/or disintegration or partial disintegration of the encapsulating material (e.g., by fracturing or melting a solid material such as, for example microcrystalline wax).

Release elements, according to the present disclosure, include tablets that encapsulate the cell extractant. Tablets to delay the release of pharmaceutical compositions are known in the art (for example, see International Patent Publication Nos. WO 97/02812 and WO 08/129517). “Tablets” is used broadly and includes microtablets, as disclosed in U.S. Patent Application No. 60/985,941 (Attorney Docket No. 63781US002), filed on Nov. 6, 2007 and entitled PROCESSING DEVICE TABLET, which is incorporated herein by reference in its entirety.

Tablets, according to the present disclosure, comprise a cell extractant admixed with an excipient. “Excipient” is used broadly to include, for example, binders, glidants (e.g., flow aids), lubricants, disintegrants, and any two or more of the foregoing. In some embodiments, tablets can comprise an outer coating, which may influence the release of an active substance (e.g., a cell extractant) when the tablet is contacted with a liquid (e.g., an aqueous liquid comprising a sample). In some embodiments, tablets can comprise fillers (e.g., a sugar such as lactose or sorbitol) as a bulking agent for the tablet. Disintegrants (e.g., a polysaccharide such as starch or cellulose) may promote wetting and/or swelling of the tablet and thereby facilitate release of the active substance when the tablet is contacted with a liquid. Sorbitol and mannitol are excipients that can promote the stability of certain cell extractants (e.g., enzymes). Mannitol can be used to delay the release of the cell extractant. In some embodiments, polyethylene glycol (PEG) is a preferred excipient to control the release of active substances from a tablet. In some embodiments, PEG compounds with molecular weights of 3300 and 8000 daltons can be used to delay the release of an active substance from a tablet.

Methods of making tablets are known in the art and include, for example, direct compression, wet granulation, dry granulation, and fluidized bed granulation.

Release elements, according to the present disclosure, include wax matrixes that encapsulate a cell extractant. In some embodiments, a plurality of bodies of cell extractant can be dispersed in a wax matrix. As the wax disintegrates (e.g., by thermal melting or mechanical disruption), the cell extractant is released from the wax. Nonlimiting examples of suitable waxes include natural or synthetic waxes or wax analogs, including paraffin wax, montan wax, carnuba wax, beeswax, scale wax, ozokerite, Utah wax, microcrystalline wax such as plastic and tank bottom derived microcrystalline waxes, wax substitutes such as Fischer-Tropsch wax, polyaklylenes such as polyethylene, polypropylene, blends and copolymers thereof.

In some embodiments, the cell extractant can be dispersed in the wax as droplets of a solution (e.g., an aqueous solution) that is immiscible with the wax. In some embodiments, the cell extractant can be dispersed in the wax as solid or semi-solid particles or agglomerates. Methods of making such dispersions of liquids or solids in wax are well known in the art.

FIG. 10 shows a perspective view of one embodiment of a release element 1040 comprising a matrix material 1092. In the illustrated embodiment, the matrix material 1092 comprises a film or block of wax. Dispersed in the matrix material 1092 are cavities 1094 comprising a cell extractant. It is recognized that the amount and/or concentration of cell extractant dispersed in the release element 1040 and the shape and dimensions of the release element 1040 can be modified for use within a given detection article.

Release elements, according to the present invention, include substrates coated with a matrix material comprising a cell extractant. Release elements comprising a coated substrate with a cell extractant are disclosed in U.S. Patent Application No. 61/175,987, filed May 6, 2009 and entitled COATED SUBSTRATES COMPRISING A CELL EXTRACTANT AND BIODETECTION METHODS THEREOF, which is incorporated herein by reference in its entirety. The matrix material can be any suitable matrix material as described herein.

Matrix materials can be coated onto a substrate using coating processes that are known in the art such as, for example, dip coating, knife coating, curtain coating, spraying, kiss coating, gravure coating, offset gravure coating, and/or printing methods such as screen printing and inkjet printing. In some embodiments, the coating can be applied in a pre-determined pattern. The choice of the coating process will be influenced by the shape and dimensions of the solid substrate and it is within the grasp of a person of ordinary skill in the appropriate art to recognize the suitable process for coating any given solid substrate.

In some embodiments, matrix material is coated onto the substrate as a pre-formed matrix (e.g., a polymer matrix) comprising a cell extractant. In some embodiments, a mixture comprising matrix precursors and cell extractant are coated onto the substrate and the matrix is formed on the substrate using, for example, polymerization processes known in the art and/or described herein. In some embodiments, a pre-formed matrix is coated onto the substrate or a matrix is formed on the substrate and, subsequently, the cell extractant is loaded into the substrate using processes known in the art and/or described herein.

In some embodiments, the coating mixture comprises an additive (e.g., a binder or viscosifier) to facilitate the coating process and/or to facilitate the adherence of the matrix material to the substrate. Non-limiting examples of additives include gums (e.g., guar gum, xanthan gum, alginates, carrageenan, pectin, agar, gellan, agarose), polysaccharides (e.g., starch, methylcellulose, carboxymethylcellulose, hydroxypropylmethylcellulose), and polypeptides (e.g., gelatin,).

Coating additives should be selected for their compatibility with the detection system used to detect cells in a sample. This compatibility can be tested by combining the additive with the detection system (e.g., luciferase and luciferin) and the analyte to be detected (e.g., ATP), measuring the response, and determining whether the additive substantially inhibits the detection of the analyte, as described herein.

The substrate onto which the matrix material is coated includes a variety of solid substrates. Nonlimiting examples of suitable substrate materials onto which matrixes comprising a cell extractant can be coated include plastic (e.g., polycarbonate, polyalkylenes such as polyethylene and polypropylene, polyesters, polyacrylates, and derivatives and blends thereof), metals (e.g., gold, graphite, platinum, palladium, nickel), glass, cellulose and cellulose derivatives (e.g., filter papers), ceramic materials, open-cell foams (e.g., polyurethane foam), nonwoven materials (e.g., membranes, PTFE membranes), and combinations thereof (e.g., a plastic-coated metal foil). The substrate can be configured in a variety of forms including, for example, fibers, nonwoven materials (e.g. nonwoven materials made from fibrous material comprising cellulose, glass, polyester, polyalkylene, polystyrene, and derivatives or combinations thereof), particles (e.g., beads), sheets, films, and membranes.

In some embodiments, the substrate can be a filter, such as Grade 4, 20-25 μm Qualitative Filter Paper, Grade 30, Glass-Fiber Filter Paper, Grade GB005, a thick (1.5 mm) highly absorbent blotting paper (all obtained from Whatman, Inc, Florham Park, N.J.), Zeta Plus Virosorb 1MDS discs (CUNO, Inc, Meriden, Conn.) and 0.45 μm MF-Millipore membrane (Millipore, Billerica, Mass.). Any one of the above substrates can be loaded a cell extractant solution containing polyvinyl alcohol. Any one of the above substrates can be loaded a cell extractant solution containing VANTOCIL (Arch Chemicals, Norwalk, Conn.). Any one of the above substrates can be loaded a cell extractant solution containing CARBOSHIELD (Lonza, Walkersville, Md.). Any one of the above substrates can be loaded a cell extractant solution containing 5% benzalkonium chloride solution.

Matrix materials, cell extractants, and substrates should be selected for their compatibility with the detection system used to detect cells in a sample. This compatibility can be tested by 1) detecting an amount of analyte in a detection system (e.g., a combining ATP with luciferin and luciferase and measuring the amount of luminescence with a luminometer, as described herein); 2) repeating the detection step with the matrix material, cell extractant, or substrate; and 3) comparing the results of step 1 with the results of step 2 to determine whether the matrix material, cell extractant, or substrate substantially inhibits the detection and/or measurement of the analyte in the reaction.

### Cell Extractants:

In some embodiments, chemical cell extractants include biochemicals, such as proteins (e.g., cytolytic peptides and enzymes). In some embodiments, the cell extractant increases the permeability of the cell, causing the release of biological analytes from the interior of the cell. In some embodiments, the cell extractant can cause or facilitate the lysis (e.g., rupture or partial rupture) of a cell.

In some embodiments, cell extractants include chemicals and mixtures of chemicals that are known in the art and include, for example, surfactants and quaternary amines, biguanides, surfactants, phenolics, cytolytic peptides, and enzymes. Typically, the cell extractant is not avidly bound (either covalently or noncovalently) to the release element and can be released from the release element when the release element is contacted with an aqueous liquid.

Surfactants generally contain both a hydrophilic group and a hydrophobic group. The release element may contain one or more surfactants selected from anionic, nonionic, cationic, ampholytic, amphoteric and zwitterionic surfactants and mixtures thereof. A surfactant that dissociates in water and releases cation and anion is termed ionic. When present, ampholytic, amphoteric and zwitterionic surfactants are generally used in combination with one or more anionic and/or nonionic surfactants. Nonlimiting examples of suitable surfactants and quaternary amines include TRITON X-100, Nonidet P-40 (NP-40), Tergitol, Sarkosyl, Tween, SDS, Igepal, Saponin, CHAPSO, benzalkonium chloride, benzethonium chloride, ‘cetrimide’ (a mixture of dodecyl-, tetradecyl- and hexadecyl-trimethylammoium bromide), cetylpyridium chloride, (meth)acrylamidoalkyltrimethylammonium salts (e.g., 3-methacrylamidopropyltrimethylammonium chloride and 3-acrylamidopropyltrimethylammonium chloride) and (meth)acryloxyalkyltrimethylammonium salts (e.g., 2-acryloxyethyltrimethylammonium chloride, 2-methacryloxyethyltrimethylammonium chloride, 3-methacryloxy-2-hydroxypropyltrimethylammonium chloride, 3-acryloxy-2-hydroxypropyltrimethylammonium chloride, and 2-acryloxyethyltrimethylammonium methyl sulfate). Other suitable monomeric quaternary amino salts include a dimethylalkylammonium group with the alkyl group having 2 to 22 carbon atoms or 2 to 20 carbon atoms. That is, the monomer includes a group of formula

—N(CH₃)₂(CₙH₂ₙ₊₁)⁺ where n is an integer having a value of 2 to 22. Exemplary monomers include, but are not limited to monomers of the following formula

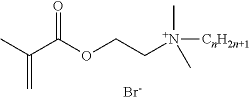

where n is an integer in the range of 2 to 22.

Non-limiting examples of suitable biguanides, which include bis-biguanides, include polyhexamethylene biguanide hydrochloride, p-chlorophenyl biguanide, 4-chloro-benzhydryl biguanide, alexidine, halogenated hexidine such as, but not limited to, chlorhexidine(1,1′-hexamethylene -bis-5-(4-chlorophenyl biguanide), and salts thereof.

Non-limiting examples of suitable phenolics include phenol, salicylic acid, 2-phenylphenol, 4-t-amylphenol, Chloroxylenol, Hexachlorophene, 4-chloro-3,5-dimethylphenol (PCMX), 2-benzyl-4-chlorophenol, triclosan, butylated hydroxytoluene, 2-Isopropyl-5-methyl phenol, 4-Nonylphenol, xylenol, bisphenol A, Orthophenyl phenol, and Phenothiazines, such as chlorpromazine, prochlorperazine and thioridizine.

Non-limiting examples of suitable cytolytic peptides include A-23187 (Calcium ionophore), Dermaseptin, Listerolysin, Ranalexin, Aerolysin, Dermatoxin, Maculatin, Ranateurin, Amphotericin B, Direct lytic factors from animal venoms, Magainin, Rugosin, Ascaphin, Diptheria toxin, Maxymin, Saponin, Aspergillus haemolysin, Distinctin, Melittin, Staphylococcus aureus toxins, (α, β, χ, δ), Alamethicin, Esculetin, Metridiolysin, Streptolysin O, Apolipoproteins, Filipin, Nigericin, Streptolysin S, ATP Translocase, Gaegurin, Nystatin, Synexin, Bombinin, GALA, Ocellatin, Surfactin, Brevinin, Gramicidin, P25, Tubulin, Buforin, Helical erythrocyte lysing peptide, Palustrin, Valinomycin, Caerin, Hemolysins, Phospholipases, Vibriolysin, Cereolysin, Ionomycin, Phylloxin, Colicins, KALA, Polyene Antibiotics, Dermadistinctin, LAGA, Polymyxin B.

Non-limiting examples of suitable enzymes include lysozyme, lysostaphin, bacteriophage lysins, achromopeptidase, labiase, mutanolysin, streptolysin, tetanolysin, a-hemolysin, lyticase, lysing enzymes from fungi, cellulase, pectinase, Driselase® Viscozyme® L, pectolyase.

In some embodiments where the release element is a hydrogel, a precursor composition from which the hydrogel is made can contain an anionic or cationic monomer, such as described in WO 20007/146722, which is incorporated herein by reference in its entirety. The anionic or cationic monomer is incorporated into the hydrogel and, as such can retain cell extractant activity. In some embodiments, the anionic or cationic monomers can be crosslinked to the surface of a hydrogel. Hydrogel beads or fibers can be dipped into a solution of the cationic monomers briefly, then quickly removed and cross-linked using actinic radiation (UV, E-beam, for example). This will result in the cationic monomer chemically bonding to the outer surface of the hydrogel beads or fibers.

In some embodiments, various combinations of cell extractants can be used in the precursor composition (from which the hydrogel is synthesized) or sorbate (which is loaded into the hydrogel after synthesis of the hydrogel). Any other known cell extractants that are compatible with the precursor compositions or the resulting hydrogels can be used. These include, but are not limited to, chlorhexidine salts such as chlorhexidine gluconate (CHG), parachlorometaxylenol (PCMX), triclosan, hexachlorophene, fatty acid monoesters and monoethers of glycerin and propylene glycol such as glycerol monolaurate, Cetyl Trimethylammonium Bromide (CTAB), glycerol monocaprylate, glycerol monocaprate, propylene glycol monolaurate, propylene glycol monocaprylate, propylene glycol moncaprate, phenols, surfactants and polymers that include a (C12-C22) hydrophobe and a quaternary ammonium group or a protonated tertiary amino group, quaternary amino-containing compounds such as quaternary silanes and polyquaternary amines such as polyhexamethylene biguanide, transition metal ions such as copper containing compounds, zinc containing compounds, and silver containing compounds such as silver metal, silver salts such as silver chloride, silver oxide and silver sulfadiazine, methyl parabens, ethyl parabens, propyl parabens, butyl parabens, octenidene, 2-bromo-2-nitropropane-1,3 diol, or mixtures of any two or more of the foregoing.

Suitable cell extractants also include dialkyl ammonium salts, including N-(n-dodecyl)-diethanolamine; cationic ethoxylated amines, including ‘Genaminox K-10’, Genaminox K-12, ‘Genamin TCL030’, and ‘Genamin C100; amidines, including propamidine and dibromopropamidine; peptide antibiotics, including polymyxin B and nisin; polyene antibiotics, including nystatin, amphotericin B, and natamycin; imidazoles, including econazole, clotramizole and miconazole; oxidizing agents, including stabilized forms of chlorine and iodine; and the cell extractants described in U.S. Pat. No. 7,422,868, which is incorporated herein by reference in its entirety.

Cell extractants are preferably chosen not to inactivate the detection system (e.g., a detection reagent such as luciferase enzyme) of the present invention. For microbes requiring harsher cell extractants (e.g., ionic detergents etc.), modified detection systems (such as luciferases exhibiting enhanced stability in the presence of these agents, such as those disclosed in U.S. Patent Application Publication No. 2003/0104507, which is hereby incorporated by reference in its entirety) are particularly preferred.

Methods of the present invention provide for the release of an effective amount of cell extractant from a release element to cause the release of biological analytes from a live cell. The present disclosure includes a variety of cell extractants known in the art and each of which may be released from the release element at a different rate and may exert its effect on living cells at a different concentration than the others. The following will provide guidance concerning the factors to be considered in selecting the cell extractant and the in determining an effective amount to include in the release element.

It is known in the art that the efficacy of any cell extractant is determined primarily by two factors—concentration and exposure time. That is, in general, the higher the concentration of a cell extractant, the greater the effect (e.g., permeabilization of the cell membrane and/or release of biological analytes from the cell) it will have on a living cell. Also, at any given concentration of cell extractant, in general, the longer you expose a living cell to the cell extractant, the greater the effect of the cell extractant. Other extrinsic factors such as, for example, pH, co-solvents, ionic strength, and temperature are known in the art to affect the efficacy of certain cell extractant. It is known that these extrinsic factors can be controlled by, for example, temperature controllers, buffers, sample preparation, and the like. These factors, as well as the cell extractant, can also have effects on the detection systems used to detect biological analytes. It is well within the grasp of a person of ordinary skill to perform a few simple experiments to determine an effective amount of cell extractant to produce the articles and perform the methods of the present disclosure. Further guidance is provided in the Examples described herein.

Initial experiments to determine the effect of various concentrations of the cell extractant on the cells and/ or the detection system can be performed. Initially, a candidate release element can be screened for its effect on the biological analyte detection system. For example, the release element can be infused with a cell extractant as described herein. Subsequently, the release element comprising the cell extractant can be placed into an ATP assay (without bacterial cells) similar to that described herein in Example 30. The assay can be run with solutions of reagent-grade ATP (e.g. from about 0.1 to about 100 picomoles of ATP) and the amount of bioluminescence emitted by the luciferase reaction in the sample with the release element can be compared to the amount of bioluminescence emitted by a sample without the release element. Preferably, the amount of bioluminescence in the sample with the release element is greater than 50% of the amount of bioluminescence in the sample without the release element. More preferably, the amount of bioluminescence in the sample with the release element is greater than 90% of the amount of bioluminescence in the sample without the release element. Most preferably, the amount of bioluminescence in the sample with the release element is greater than 95% of the amount bioluminescence in the sample without the release element.

Additionally, the effect of the cell extractant on the release of the biological analyte from the cells can be determined experimentally, similar to that described in Example 21. For example, liquid suspensions of cells (e.g., microbial cells such as Staphylococcus aureus) are exposed to relatively broad range of concentrations of a cell extractant (e.g., BARDAC 205M) for a period of time (e.g. up to several minutes) in the present of a detection system to detect biological analytes from a cell (e.g., an ATP detection system comprising luciferin, luciferase, and a buffer at about pH 7.6 to 7.8). The biological analyte is measured periodically, with the first measurement usually performed immediately after the cell extractant is added to the mixture, to determine whether the release of the biological analyte (in this example, ATP) from the cells can be detected. The results can indicate the optimal conditions (i.e., liquid concentration of cell extractant and exposure time) to detect the biological analyte released from the cells. As shown in Table 26, the results can also indicate that, at higher concentrations of cell extractant, the cell extractant may be less effective in releasing the biological analyte (e.g., ATP) and/or may interfere with the detection system (i.e., may absorb the light or color generated by the detection reagents).

After the effective amount of cell extractant in liquid mixtures is determined, consideration should be given to the amount of cell extractant to incorporate into the release element by the methods described herein. When the release element contacts a liquid mixture (e.g., a sample suspected of containing live cells in an aqueous suspension) the cell extractant can be released from the release element (e.g., by diffusion) and the concentration of the cell extractant in the liquid mixture increases until an equilibrium is reached. Without being bound by theory, it can be assumed that, until the equilibrium is reached, a concentration gradient of cell extractant will exist in the liquid, with a higher concentration of extractant present in the portion of the liquid proximal the release element. When the concentration of the cell extractant reaches an effective concentration in a portion of the liquid containing a cell, the cell releases biological analytes. The released biological analytes are thereby available for detection by a detection system.

Achieving an effective concentration of cell extractant in the liquid containing the sample can be controlled by several factors. For example, the amount of cell extractant loaded into the release element can affect final concentration of cell extractant in the liquid at equilibrium. Additionally, the amount of release element and, in some embodiments, the amount of surface area of the release element in the liquid mixture can affect the rate of release of the cell extractant from the release element and the final concentration of cell extractant in the liquid at equilibrium. Furthermore, the temperature of the aqueous medium can affect the rate at which the release element releases the cell extractant. Other factors, such as the ionic properties and or hydrophobic properties of the cell extractant and the release element may affect the amount of cell extractant released from the release element and the rate at which the cell extractant is released from the release element. All of these factors can be optimized with routine experimentation by a person of ordinary skill to achieve the desired parameters (e.g., manufacturing considerations for the articles and the time-to-result for the methods) for detection of cells in a sample. In general, it is desirable to incorporate at least enough cell extractant into the release element to achieve the effective amount (determined by the experimentation using the cell extractant without a release element) when the cell extractant reaches equilibrium between the release element and the volume of liquid comprising the sample material. It may be desirable to add a larger amount of cell extractant to the release element (than the amount determined by experimentation using the cell extractant without a release element) to reduce the amount of time it take for the release element to release an effective amount of cell extractant.

In some embodiments wherein the release element comprises a matrix, the cell extractant can diffuse into the matrix, diffuse out of the matrix, or both. The rate of diffusion should be controllable by, for example, varying the matrix material and/or the crosslink density, by varying the polar solvent in which the matrix is made, by varying the solubility of the cell extractant in the polar solvent in which the matrix is made, and/or by varying the molecular weight of the cell extractant. The rate of diffusion can also be modified by varying the shape, size, and surface topography of the matrix.

Without being bound by theory, it is believed that migration of the cell extractant out of the release element can occur spontaneously (e.g., by diffusion) upon contact of the release element and a liquid (e.g., an aqueous liquid comprising a sample). In some embodiments, migration of the cell extractant out of the release element can be facilitated.

In some embodiments, migration of the cell extractant out of the release element is facilitated by providing a chemical facilitator. The chemical facilitator can be, for example, an acid or a base. Changing the pH of the mixture may disrupt ionic interaction between the release element and the cell extractant, thereby facilitating the migration of the cell extractant out of the release element. PCT International Publication No. WO2005/094792 entitled ANIONIC HYDROGEL MATRICES WITH PH DEPENDENT MODIFIED RELEASE AS DRUG CARRIERS, which is incorporated herein by reference in its entirety, discloses hydrogel compositions with pH dependent modified release of drugs or disinfectants. In some embodiments, migration of the cell extractant can be facilitated by changing the ionic strength of the liquid (e.g., by adding or removing a salt).

In some embodiments, migration of the cell extractant out of the release element is facilitated by a mechanical process. Non-limiting examples of suitable mechanical processes include vibrating, stirring, or compressing the release element.

The release element can be contacted with the liquid sample material either statically, dynamically (i.e., with mixing by vibration, stirring, aeration or compressing, for example), or a combination thereof. Example 16 shows that mixing can cause a faster release of an effective amount of cell extractant from a release element. Example 17 shows that compressing the release element can, in some embodiments, cause a faster release of an effective amount of cell extractant from release element. Compressing the release element can include, for example, pressing the release element against a surface and/or crushing the release element. Thus, in some embodiments, mixing can advantageously provide a faster release of cell extractant and thereby a faster detection of biological analytes (e.g., from live cells) in a sample. In some embodiments, compressing the release element (e.g., by exerting pressure against the release element using a sample acquisition device such as a swab or a spatula, a carrier (described below) or some other suitable implement) can advantageously provide a faster release of cell extractant and thereby a faster detection of biological analytes in a sample. Additionally, the step of compressing the release element can be performed to accelerate the release of the cell extractant at a time that is convenient for the operator.

In some embodiments, static contact can delay the release of an effective amount of cell extractant and thereby provide additional time for the operator to carry out other procedures (e.g., reagent additions, instrument calibration, and/or specimen transport) before detecting the biological analytes. In some embodiments, it may be advantageous to hold the mixture statically until a first biological analyte measurement is taken and then dynamically mix the sample to reduce the time necessary to release an effective amount of cell extractant.

It is fully anticipated that the most preferred concentration(s) or concentration range(s) functional in the methods of the invention will vary for different microbes and for different cell extractants and may be empirically determined using the methods described herein or commonly known to those skilled in the art.

### Samples and Sample Acquisition Devices:

Articles and methods of the present disclosure provide for the detection of biological analytes in a sample. In some embodiments, the articles and methods provide for the detection of biological analytes from live cells in a sample. In certain preferred embodiments, the articles and methods provide for the detection of live microbial cells in a sample. In certain preferred embodiments, the articles and methods provide for the detection of live bacterial cells in a sample.

The term “sample” as used herein, is used in its broadest sense. A sample is a composition suspected of containing a biological analyte (e.g., ATP) that is analyzed using the invention. While often a sample is known to contain or suspected of containing a cell or a population of cells, optionally in a growth media, or a cell lysate, a sample may also be a solid surface, (e.g., a swab, membrane, filter, particle), suspected of containing an attached cell or population of cells. It is contemplated that for such a solid sample, an aqueous sample is made by contacting the solid with a liquid (e.g., an aqueous solution) which can be mixed with hydrogels of the present. Filtration of the sample is desirable in some cases to generate a sample, e.g., in testing a liquid or gaseous sample by a process of the invention. Filtration is preferred when a sample is taken from a large volume of a dilute gas or liquid. The filtrate can be contacted with hydrogels of the present disclosure, for example after the filtrate has been suspended in a liquid.

Suitable samples include samples of solid materials (e.g., particulates, filters), semisolid materials (e.g., a gel, a liquid suspension of solids, or a slurry), a liquid, or combinations thereof. Suitable samples further include surface residues comprising solids, liquids, or combinations thereof. Non-limiting examples of surface residues include residues from environmental surfaces (e.g., floors, walls, ceilings, fomites, equipment, water, and water containers, air filters), food surfaces (e.g., vegetable, fruit, and meat surfaces), food processing surfaces (e.g., food processing equipment and cutting boards), and clinical surfaces (e.g., tissue samples, skin and mucous membranes).

The collection of sample materials, including surface residues, for the detection of biological analytes is known in the art. Various sample acquisition devices, including spatulas, sponges, swabs and the like have been described. The present disclosure provides sample acquisition devices with unique features and utility, as described herein.

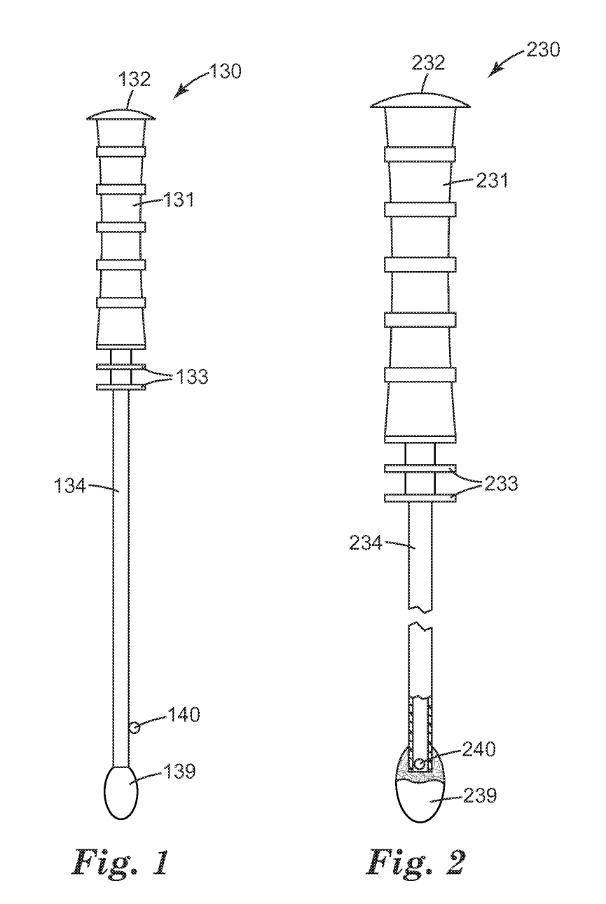

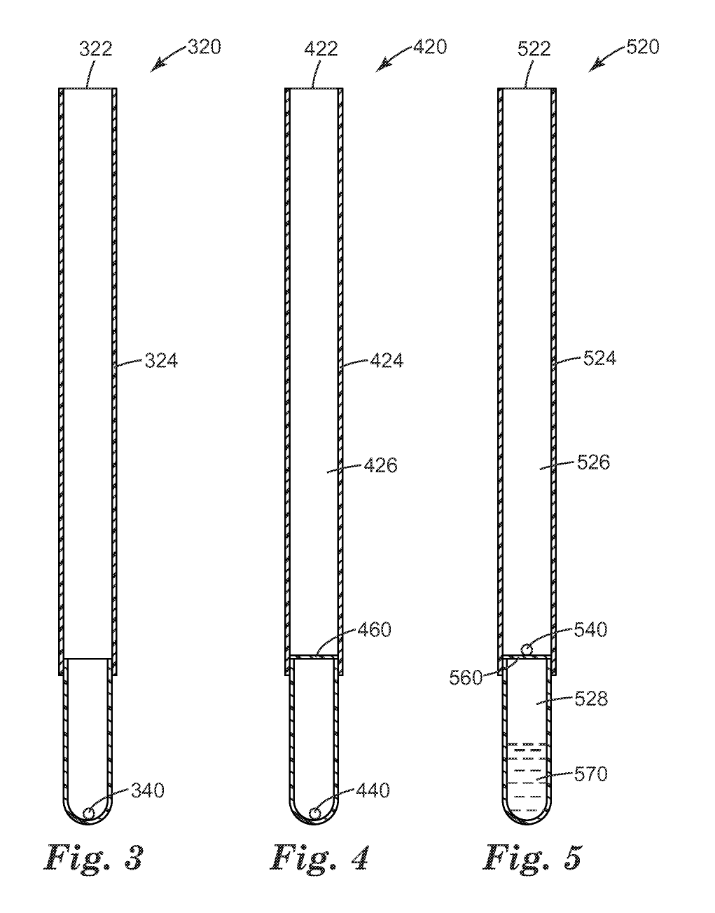

Turning now to the Figures, FIG. 1 shows a side view of one embodiment of a sample acquisition device 130 according to the present disclosure. The sample acquisition device 130 comprises a handle 131 which can be grasped by the operator while collecting a sample. The handle comprises an end 132 and, optionally, a plurality of securing members 133. Securing members 133 can be proportioned to slideably fit into a housing (such as housing 320 or housing 420 shown in FIGS. 3 and 4, for example). In some embodiments, the securing members 133 can form a liquid-resistant seal to resist the leakage of fluids from a housing.

The sample acquisition device 130 further comprises an elongated shaft 134 and a tip 139. In some embodiments, the shaft 134 can be hollow. The shaft 134 comprises a tip 139, positioned near the end of the shaft 134 opposite the handle 131. The tip 139 can be used to collect sample material and can be constructed from porous materials, such as fibers (e.g., rayon or Dacron fibers) or foams (e.g., polyurethane foam) which can be affixed to the shaft 134. In some embodiments, the tip 139 can be a molded tip as described in U.S. Patent Application No. 61/029,063, filed on Dec. 5, 2007 and entitled, “SAMPLE ACQUISITION DEVICE”, which is incorporated herein by reference in its entirety. The construction of sample acquisition devices 130 is known in the art and can be found, for example, in U.S. Pat. No. 5,266,266, which is incorporated herein by reference in their entirety.

Optionally, the sample acquisition device 130 can further comprise a release element 140. In some embodiments, the release element 140 is positioned in or on the sample acquisition device 130 at a location other than the tip 139 that is used to collect the sample (e.g., on the shaft 134, as shown in FIG. 1). In some embodiments, the release element 140 can be positioned on an exterior surface of the sample acquisition device 130 (as shown in FIG. 1). In some embodiments, the release element 140 can be positioned on an interior surface of the sample acquisition device 130 (as shown in FIG. 2). The release element 140 can be coated onto shaft 134 as described herein or it can be adhered to the shaft 134 by, for example, a pressure-sensitive adhesive or a water-soluble adhesive (not shown). The adhesive should be selected for its compatibility with the detection system used to detect a biological analyte from live cells (i.e., the adhesive should not significantly impair the accuracy or sensitivity of the detection system).

In use, the tip 139 of a sample acquisition device 130 is contacted with a sample material (e.g., a solid, a semisolid, a liquid suspension, a slurry, a liquid, a surface, and the like) to obtain a sample suspected of containing cells. The sample acquisition device 130 can be used to transfer the sample to a detection system as described herein.

FIG. 2 shows a partial cross-sectional view of another embodiment of a sample acquisition device 230 according to the present disclosure. In this embodiment, the sample acquisition device 230 comprises a handle 231 with an end 232, optional securing members 233 to slideably fit within a housing (not shown), a hollow elongated shaft 234, and a tip 239 comprising porous material. The sample acquisition device 230 further comprises a release element 240, which comprises a cell extractant, disposed in the interior portion of the shaft 234. Thus, the sample acquisition device 230 provides an enclosure (shaft 234) containing the release element 240. The material comprising the tip 239 is porous enough to permit liquids to flow freely into the interior of the shaft 234 without permitting the release element 240 to pass through the material and out of the tip 239.

In use, sample acquisition device 230 can be used to contact surfaces, preferably dry surfaces, to obtain sample material. After the sample is obtained, the tip 239 of the sample acquisition device 230 is moistened with a liquid (e.g. water or a buffer; optionally, including a detection reagent such as an enzyme and/or an enzyme substrate), thereby permitting an effective amount of the cell extractant to be released from the release element 240 and to contact the sample material. The release of an effective amount of cell extractant from release element 240 permits the sample acquisition device 230 to be used in methods to detect biological analytes from live cells as described herein.

Another embodiment (not shown) of a sample acquisition device including a release element can be derived from the “Specimen Test Unit” disclosed by Nason in U.S. Pat. No. 5,266,266 (hereinafter, referred to as the “Nason patent”). In particular, referring to FIGS. 7-9 of the Nason patent, the handle of the sample acquisition devices described herein can be modified to embody Nason's functional elements of the housing base 14 (which forms reagent chamber 36) and the seal fitting 48, which includes central passage 50 (optional, with housing cap 30) connected to the hollow swab shaft 22. The central passage 50 of the seal fitting 48 can be closed by a break-off nib 52 in the form of an extended rod segment 54 connected to the seal fitting 48 at the inboard end of the passage 50 via a reduced diameter score 56. Thus, in one embodiment of the present disclosure, the sample acquisition device handle comprises a reagent chamber, as described by Nason. The reagent chamber located in the handle of the sample acquisition device of this embodiment includes release element particles (e.g., beads). Thus, the sample acquisition device of this embodiment provides an enclosure (reagent chamber 36) containing the release element. In this embodiment, the release element particles are not suspended in a liquid medium that causes the release of the cell extractant from the release element. The release element particles are proportioned and shaped to allow free passage of the individual particles into and through the central passage 50 and the hollow shaft 22.

In use, the sample acquisition device comprising a handle including a reagent chamber can be used to obtain a sample as described herein. If the sample is a liquid, the break-off nib 52 can be actuated, as described in the Nason patent, enabling the passage of the release element through the shaft to contact the liquid sample in the swab tip, thereby forming a liquid mixture comprising the sample and the release element. The liquid mixture comprising the sample and the release element can be used for the detection of a biological analyte associated with a live cell, as described herein. If the sample is a solid or semi-solid, the tip of the sample acquisition device can be contacted or submersed in a liquid solution and the break-off nib 52 can be actuated, as described in the Nason patent, enabling the passage of the release element through the shaft to contact the liquid sample in the swab tip, thereby forming a liquid mixture comprising the sample and the release element. The liquid mixture comprising the sample and the release element can be used for the detection of a biological analyte associated with a live cell, as described herein.

### Detection Devices:

FIG. 3 shows a cross-sectional view of one embodiment of a housing 320 of a detection device according to the present disclosure. The housing 320 comprises an opening 322 configured to receive a sample acquisition device and at least one wall 324. Disposed in the housing 320 is a release element 340. Thus, the housing 320 provides an enclosure containing the release element 340.

In FIG. 3, the release element 340 is a shaped hydrogel, in the form of a generally spherical bead. It will be appreciated that a bead is just one example of a variety shaped release elements disclosed herein that are suitable for use in housing 320.

In some embodiments (not shown), the release element 340 can be coated onto a solid substrate (e.g., the wall 324 of the housing 320). Nonlimiting examples of other suitable solid substrates (not shown) onto which release elements 340 of the present disclosure can be coated include a polymeric film, a fiber, a nonwoven material, a ceramic particle, paper, and a polymeric bead. Solid substrates can be coated with release element 340 by a variety of processes including; for example, dip coating, knife coating, curtain coating, spraying, kiss coating, gravure coating, offset gravure coating, and/or printing methods such as screen printing and inkjet printing can be used to apply the composition onto the substrate in a pattern if desired. The choice of the coating process will be influenced by the shape and dimensions of the solid substrate and it is within the grasp of a person of ordinary skill in the appropriate art to recognize the suitable process for coating any given solid substrate.

It should be recognized that in this and all other embodiments (for example, the illustrated embodiments of FIGS. 1, 2, 4, 5, 6A-B, 7, and 8), the release element (e.g., release element 340) may include a plurality (for example, at least 2, 3, 4, 5, up to 10, up to 20, up to 50, up to 100, up to 500, up to 1000) of release element bodies such as beads, fibers, ribbons, coated substrates, or the like. For example, release element 340 can comprise up to 2, up to 3, up to 4, up to 5, up to 10, up to 20, up to 50, up to 100, up to 500, up to 1000 or more release element bodies.

The wall 324 of the housing 320 can be cylindrical, for example. It will be appreciated that other useful geometries, some including a plurality of walls 324, are possible and within the grasp of one of ordinary skill in the appropriate art. The housing 320 can be constructed from a variety of materials such as plastic (e.g., polypropylene, polyethylene, polycarbonate) or glass. Preferably, at least a portion of the housing 320 is constructed from materials that have optical properties that allow the transmission of light (e.g., visible light). Suitable materials are well known in devices used for biochemical assays such as ATP tests, for example.

Optionally, housing 320 can comprise a cap (not shown) that can be shaped and dimensioned to cover the opening 322 of the housing 320. It should be recognized that other housings (for example, housings 420 and 520 as shown in FIGS. 4 and 5, respectively and described herein) can also comprise a cap.

In some embodiments, the housing 320 can be used in conjunction with a sample acquisition device (not shown). Optionally, the sample acquisition device may comprise a release element, such as, for example, sample acquisition devices 130 or 230 shown in FIGS. 1 and 2, respectively, and described herein. The release element in the sample acquisition device can comprise the same composition and/or amount of cell extractant as release element 340. The release element in the sample acquisition device can comprise a different composition and/or amount of cell extractant than release element 340. In some embodiments, the sample acquisition device can comprise a somatic cell extractant and the housing 320 can comprise a microbial cell extractant. In some embodiments, the sample acquisition device can comprise a microbial cell extractant and the housing 320 can comprise a somatic cell extractant. It should be recognized that other housings (for example, housings 420 and 520 as shown in FIGS. 4 and 5, respectively and described herein) can similarly comprise a sample acquisition device that may optionally include a release element.

The housing 320 can be used in methods to detect live cells in a sample. During use, the operator can form a liquid (e.g., an aqueous liquid or aqueous solutions containing glycols and/or alcohols) mixture in the housing 320, the mixture comprising a liquid sample and the release element 340. In some embodiments, the mixture can further comprise a detection reagent. The liquid mixture comprising the sample and the release element 440 can be used for the detection of a biological analyte associated with a live microorganism.

FIG. 4 shows a partial cross-section view of one embodiment of a housing 420 of a detection device according to the present disclosure. The housing 420 comprises a wall 424 with an opening 422 configured to receive a sample acquisition device. A frangible seal 460 divides that housing 420 into two portions, the upper compartment 426 and the reaction well 428. Disposed in the reaction well 428 is a release element 440. Thus, the housing 420 provides an enclosure containing the release element 440.

The frangible seal 460 forms a barrier between the upper compartment 426 (which includes the opening 422 of the housing 420) and the reaction well 428. In some embodiments, the frangible seal 460 forms a water-resistant barrier. The frangible seal 460 can be constructed from a variety of frangible materials including, for example polymer films, metal-coated polymer films, metal foils, dissolvable films (e.g., films made of low molecular weight polyvinyl alcohol or hydroxypropyl cellulose (HPC) and combinations thereof.

Frangible seal 460 may be connected to the wall 424 of the housing 420 using a variety of techniques. Suitable techniques for attaching a frangible seal 460 to a wall 424 include, but are not limited to, ultrasonic welding, any thermal bonding technique (e.g., heat and/or pressure applied to melt a portion of the wall 424, the frangible seal 460, or both), adhesive bonding, stapling, and stitching. In one desired embodiment of the present invention, the frangible seal 460 is attached to the wall 424 using an ultrasonic welding process.

The housing 420 can be used in methods to detect cells in a sample. Methods of the present disclosure include the formation of a liquid mixture comprising the sample material and the release element 440 and include the detection of a biological analyte, as described herein.

If the sample is a liquid sample (e.g., water, juice, milk, meat juice, vegetable wash, food extracts, body fluids and secretions, saliva, wound exudate, and blood), the liquid sample can be transferred (e.g., poured or pipetted) directly into the upper compartment 426. A detection reagent can be added to the sample before the sample is transferred to the housing 420. A detection reagent can be added to the sample after the sample is transferred to the housing 420. A detection reagent can be added to the sample while the sample is transferred to the housing 420. The frangible seal 460 can be ruptured (e.g., by piercing it with a pipette tip or a sample acquisition device) before the liquid sample is transferred to the housing 420. The frangible seal 460 can be ruptured after the liquid sample is transferred to the housing 420. The frangible seal 460 can be ruptured while the liquid sample is transferred to the housing 420. When the liquid sample is in the housing 420 and the frangible seal is ruptured, a liquid mixture comprising the sample and the release element 440 is formed. The liquid mixture comprising the sample and the release element 440 can be used for the detection of a biological analyte associated with a live microorganism.

If the sample is a solid sample (e.g., powder, particulates, semi-solids, residue collected on a sample acquisition device, air filter), the housing 420 can advantageously be used as a vessel in which the sample can be mixed with a liquid suspending medium such as, for example, water or a buffer. Preferably, the liquid suspending medium is substantially free of microorganisms. More preferably, the liquid suspending medium is sterile. Before, after or during the process of mixing the solid sample with the liquid suspending medium, a detection reagent can be added to the liquid suspending medium. Either before, after, or during the process of mixing the solid sample with the liquid suspending medium, the frangible seal 460 can be ruptured (e.g., by piercing with a pipette tip or a swab), thus forming a liquid mixture comprising the sample and the release element 440. The liquid mixture comprising the sample and the release element 440 can be used in a method for the detection of a biological analyte associated with a live cell.

FIG. 5 shows a partial cross-section view of one embodiment of a housing 520 of a detection device according to the present disclosure. The housing 520 comprises a wall 524 with an opening 522 configured to receive a sample acquisition device. A frangible seal 560 divides the housing 520 into two portions, the upper compartment 526 and the reaction well 528. Disposed in the upper compartment 526 is a release element 540. The reaction well 528 further includes a detection reagent 570.

In FIG. 5, the release element 540 is positioned on the frangible seal 560, in the upper compartment 526 of the housing 520. Thus, the housing 520 provides and enclosure containing the release element 540. In some embodiments (not shown), the release element 540 may be coupled to the frangible seal 560 or wall 524 of the upper compartment 526. For example, the release element 540 may be adhesively coupled (e.g., via a pressure-sensitive adhesive or water-soluble adhesive) or coated onto one of the surfaces (e.g., the frangible seal 560 and/or the wall 524) that form a portion of the upper compartment 526 of the housing 520.

The reagent well 528 of housing 520 comprises a detection reagent 570. Optionally, the detection reagent 570 can comprise a detection reagent (i.e., a detection reagent may be dissolved and/or suspended in the detection reagent 570). In other embodiments (not shown), the reagent well 528 can comprise a dry detection reagent (e.g., a powder, particles, microparticles, a tablet, a pellet, and the like) instead of the detection reagent 570.

The housing 520 can be used in methods to detect cells in a sample. Methods of the present disclosure include the formation of a liquid mixture comprising the sample material and the release element 440 and include the detection of a biological analyte, as described herein.

If the sample is a liquid sample (e.g., water, juice, milk, meat juice, vegetable wash, food extracts, body fluids and secretions, saliva, wound exudate, and blood), the liquid sample can be transferred (e.g., poured or pipetted) directly into the upper compartment 526, thus forming a liquid mixture comprising the sample and the release element 540. Before, after or during the transfer of the sample into the housing 520, a detection reagent can be added to the liquid sample. Before, after, or during the transfer of the liquid sample to the housing 520, the frangible seal 560 can be ruptured (e.g., by piercing with a pipette tip or a swab). The liquid mixture comprising the sample and the release element 540 can be used for the detection of a biological analyte associated with a live microorganism before and/or after the frangible seal 560 is ruptured.

If the sample is a solid sample (e.g., powder, particulates, semi-solids, residue collected on a sample acquisition device), the housing 520 can advantageously be used as a vessel in which the sample can be mixed with a liquid suspending medium such as, for example, water or a buffer. Preferably, the liquid suspending medium is substantially free of microorganisms. More preferably, the liquid suspending medium is sterile.

Mixing the solid sample with a liquid suspending medium forms a liquid mixture comprising the sample and the release element 540. Before, after or during the process of mixing the solid sample with the liquid suspending medium, a detection reagent can be added to the liquid suspending medium. Before, after, or during the process of mixing the solid sample with the liquid suspending medium, the frangible seal 560 can be ruptured (e.g., by piercing with a pipette tip or a swab). The liquid mixture comprising the sample and the release element 540 can be used for the detection of a biological analyte associated with a live microorganism, as described herein.

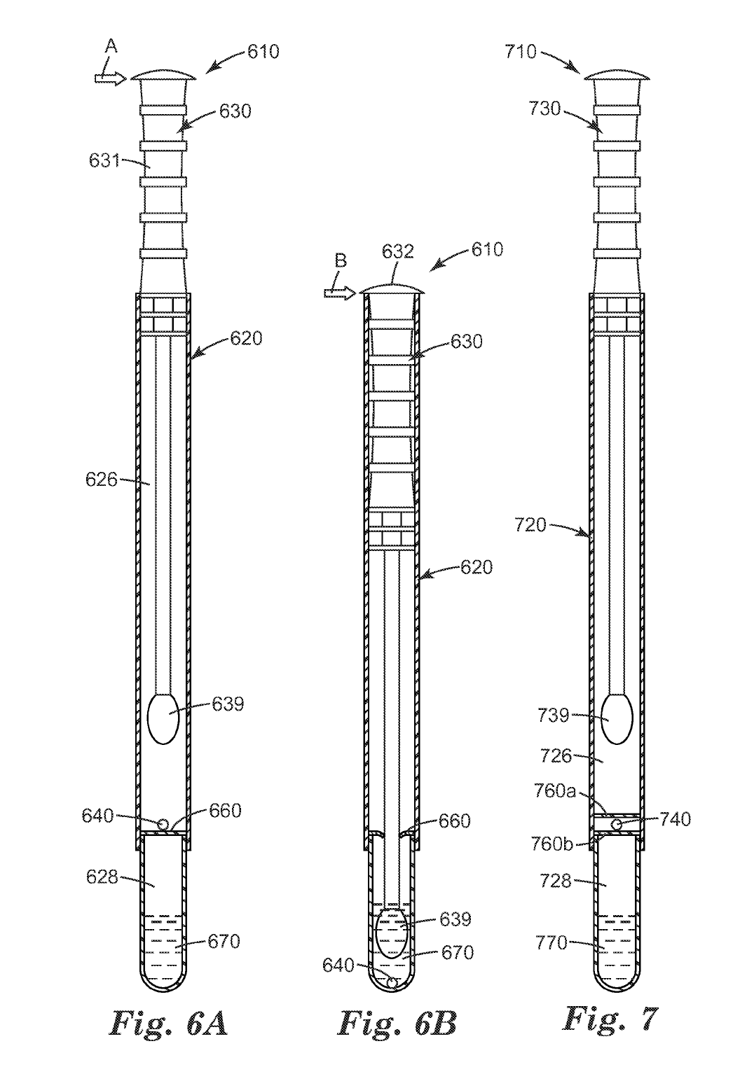

FIGS. 6A-6B show partial cross-section views of a detection device 610 according to the present disclosure. Referring to FIG. 6A, the detection device 610 comprises a housing 620 and a sample acquisition device 630, as described herein. The housing 620 includes a frangible seal 660, a release element 640 disposed in the upper compartment 626, and an optional detection reagent 670 disposed in the reagent well 628. Thus, the housing 620 provides an enclosure containing the release element 640. The detection reagent 670 may further comprise a detection reagent.

The sample acquisition device 630 comprises a handle 631 which can be grasped by the operator while collecting a sample. The sample acquisition device 630 is shown in FIG. 6A in a first position “A”, with the handle 631 substantially extending outside the housing 620. Generally, the handle 631 will be in position “A” during storage of detection device 610. During use, the sample acquisition device 630 is withdrawn from the housing 620 and the tip 629 is contacted with the area or material from which a sample is to be taken. After collecting the sample, the sample acquisition device is reinserted into the housing 620 and, typically, while the housing 620 is held in place, the end 632 of the handle 631 is urged (e.g., with finger pressure) toward the housing 620, moving the sample acquisition device 630 approximately into position “B” and thereby causing the tip 639 to pass through the frangible seal 660 and into the detection reagent 670, if present, in the reaction well 628 (as shown in FIG. 6B). As the tip 639 ruptures the frangible seal 660, the release element 640 is also moved into the reaction well 628. This process forms a liquid mixture that includes a sample and the release element 640. The liquid mixture comprising the sample and the cell extractant can be used for the detection of a biological analyte associated with a live cell, as described herein.

FIG. 7 shows a cross-sectional view of a detection device 710 comprising a housing 720 and a sample acquisition device 730, as described herein. The housing 720 is divided into an upper compartment 726 and a reaction well 728 by frangible seals 760a and 760b. Positioned between frangible seals 760a and 760b is release element 740. Thus, the housing 720 provides an enclosure containing the release element 740. Reaction well 728 comprises a detection reagent 770.

In use, the tip 739 of a sample acquisition device 730 is contacted with a sample material (e.g., a solid, a semisolid, a liquid suspension, a slurry, a liquid, a surface, and the like), as described above. After collecting the sample, the sample acquisition device 730 is reinserted into the housing 720 and the handle is urged into the housing 720, as described above, thereby causing the tip 739 to pass through frangible seals 760a and 760b and into the detection reagent in the reaction well 728. As the tip 739 passes through frangible seals 760a and 760b, the release element 740 is also moved into the detection reagent 770 in the reaction well 728. This process forms a liquid mixture that includes a sample and a release element 740. The liquid mixture comprising the sample and the cell extractant can be used for the detection of a biological analyte associated with a live microorganism, as described herein.

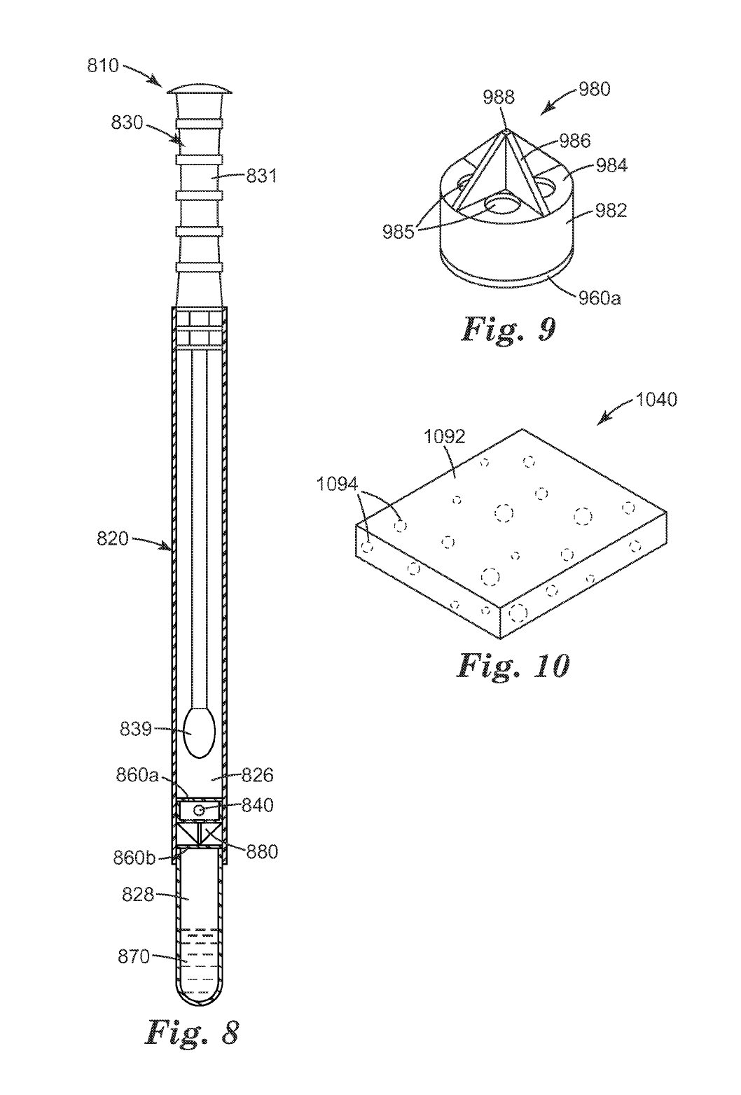

FIG. 8 shows a partial cross-section view of a detection device 810 according to the present disclosure. The detection device 810 comprises a housing 820 and a sample acquisition device 830, both as described herein. A frangible seal 860b, as described herein, divides the housing into two sections, the upper compartment 826 and the reagent chamber 828. The reagent chamber 828 includes a detection reagent 870, which may be a liquid detection reagent 870 (as shown) or a dry detection reagent as described herein. Slideably disposed in the upper compartment 826, proximal the frangible seal 860b, is a carrier 880. The carrier 880 includes a release element 840 and an optional frangible seal 860a. Thus, the carrier 880 provides an enclosure containing the release element 840. The carrier 880 can be, for example, constructed from molded plastic (e.g., polypropylene or polyethylene). In the illustrated embodiment, the frangible seal 860a functions to hold the release element 840 (shown as a hydrogel bead) in the carrier 880 during storage and handling. In some embodiments, the release element 840 is coated onto the carrier 880 and the frangible seal 860a may not be required to retain the release element 840 during storage and handling. In an alternative embodiment (not shown), release element 840 can be positioned on frangible seal 860b, rather than in the conveyor 880. In this embodiment, the conveyor 880 or the tip 839 of the sample acquisition device 830 can be used to puncture the frangible seal 860b and cause the release element 840 to drop into the reagent chamber 828.

In use, the sample acquisition device 830 is removed from the detection device 810 and a sample is collected as described herein on the tip 839. The sample acquisition device 830 is reinserted into the housing 820 and the handle 831 is urged into the housing 820, as described for the detection device in FIG. 6A-B. The tip 839 of the sample acquisition device 830 ruptures frangible seal 860A, if present, and pushes the carrier 880 through frangible seal 860b. The carrier 880 drops into the detection reagent 870 as the tip 839 comprising the sample contacts the detection reagent 870, thereby forming a liquid mixture including the sample and a release element 840. The liquid mixture comprising the sample and the release element 840 can be used for the detection of a biological analyte associated with a live cell, as described herein.

FIG. 9 shows a bottom perspective view of one embodiment of the carrier 980 of FIG. 8. The carrier 980 comprises a cylindrical wall 982 and a base 984. The wall 982 is shaped and proportioned to slideably fit into a housing (not shown). The carrier 980 further comprises optional frangible seal 960a. The base 984 comprises holes 985 and piercing members 986, which form a piercing point 988. The piercing point 988 can facilitate the rupture of a frangible seal in a housing (not shown)

Devices of the present disclosure may include a detection system. In some embodiments, the detection system comprises a detection reagent, such as an enzyme or an enzyme substrate. In certain embodiments, the detection reagent can be used for detecting ATP. The detection reagent may be loaded into a delivery element. Such delivery elements can be used conveniently to store and/or deliver the detection reagent to a liquid mixture, comprising a sample and a cell extractant, for the detection of live cells in the sample.

Delivery elements, as used herein, include encapsulating agents, matrixes, shell structures with a core, and coated substrates, as described herein. A detection reagent comprising a protein, such as an enzyme or an antibody, can be incorporated into the delivery element using the similar processes as those described for the incorporation of cell extractants into a release element. For example, luciferase can be incorporated into a delivery element during the synthesis of a polymer matrix, as described in Preparative Example 4 below. An enzyme can be incorporated into a delivery element after the synthesis of the delivery element. For example, luciferase can be incorporated into a polymer matrix delivery element as described in Preparative Example 8 below.

An enzyme substrate can be incorporated into a delivery element during the synthesis of the delivery element. For example, luciferin can be incorporated into a delivery element during the synthesis of a polymer matrix delivery element, as described in Preparative Example 3 below. An enzyme substrate can be incorporated into a delivery element after the synthesis of the delivery element. For example, luciferin can be incorporated into a polymer matrix delivery element as described in Preparative Example 7 below.

Although proteins may be incorporated into a delivery element (e.g., a hydrogel) during the synthesis of the delivery element, chemicals and or processes (e.g., u.v. curing processes) used in the synthesis process (e.g., polymerization) can potentially cause the loss of some biological activity by certain proteins (e.g. certain enzymes or binding proteins such as antibodies). In contrast, incorporation (e.g., by diffusion) of a detection reagent protein into the delivery element after synthesis of the delivery element can lead to improved retention of the protein's biological activity.

In some applications, it may be desirable that the delivery element containing a detection reagent is in a dry or partially-dried state. Certain delivery elements (e.g., swollen hydrogels) can be dried, for example, by methods known to those skilled in the art, including evaporative processes, drying in convection ovens, microwave ovens, and vacuum ovens as well as freeze-drying. When the dried delivery element is exposed to a liquid or aqueous solution, the detection reagent can diffuse out of the delivery element. The detection reagent can remain essentially dormant in the delivery element until exposed to a liquid or aqueous solution. That is, the detection reagent can be stored within the dry or partially-dried delivery element until the element is exposed to a liquid. This can prevent the waste or loss of the detection reagent when not needed and may improve the stability of moisture sensitive detection reagents that may degrade by hydrolysis, oxidation, or other mechanisms.

### Methods of Detecting Biological Analytes:

Methods of the present disclosure include methods for the detection of biological analytes that are released from live cells including, for example, live microorganisms, after exposure to an effective amount of cell extractant.

Methods of the present disclosure allow an operator instantaneously to form a liquid mixture containing a sample and a release element. In some embodiments, contact of the release element with the liquid mixture triggers the release (e.g., by diffusion) of the cell extractant from the release element into the bulk liquid. Advantageously, in some embodiments, the release of the cell extractant from the release element is triggered by a factor and/or a process step causing the release of the cell extractant. Non-limiting examples of a factor causing the release of the cell extractant include a base, an acid, and an enzyme or a chemical to solublize the release element. Non-limiting examples of processes causing the release of the cell extractant include raising or lowering the pH of the liquid sample, increasing or decreasing the concentration of a salt or a metal ion, adding an enzyme or chemical to solublize the release factor, mechanically disrupting (e.g., compressing or crushing) the release element, and thermally disrupting (e.g., freezing, freeze-thawing, or melting) the release element.

In some embodiments, the methods provide for the operator to, within a predetermined period of time after the liquid mixture is formed, measure the amount of a biological analyte in the mixture to determine the amount of acellular biological analyte in the sample. In some embodiments, the methods provide for the operator to, after a predetermined period of time during which an effective amount of cell extractant is released from the release element into the liquid mixture, measure the amount of a biological analyte to determine the amount of biological analyte from acellular material and live cells in the sample. In some embodiments, the methods provide for the operator, within a first predetermined period of time, to perform a first measurement of the amount of a biological analyte and, within a second predetermined period of time during which an effective amount of cell extractant is released from the release element, perform a second measurement of the amount of biological analyte to detect the presence of live cells in the sample. In some embodiments, the methods can allow the operator to distinguish whether biological analyte in the sample was released from live plant or animal cells or whether it was released from live microbial cells (e.g., bacteria). The present invention is capable of use by operators under the relatively harsh field environment of institutional food preparation services, health care environments and the like.

The detection of the biological analytes involves the use of a detection system. Detection systems for certain biological analytes such as a nucleotide (e.g., ATP), a polynucleotide (e.g., DNA or RNA) or an enzyme (e.g., NADH dehydrogenase or adenylate kinase) are known in the art and can be used according to the present disclosure. Methods of the present disclosure include known detections systems for detecting a biological analyte. Preferably, the accuracy and sensitivity of the detection system is not significantly reduced by the cell extractant. More preferably, the detection system comprises a homogeneous assay.

In some embodiments, the detection system comprises a detection reagent. Detection reagents include, for example, dyes, enzymes, enzyme substrates, binding partners (e.g., an antibody, a monoclonal antibody, a lectin, a receptor), and/or cofactors. In some embodiments, a detection reagent is used for detecting ATP. In some embodiments, the detection system comprises an instrument. Nonlimiting examples of detection instruments include a spectrophotometer, a luminometer, a fluorometer, a plate reader, a thermocycler, an incubator. Thus an analyte associated with a cell (e.g., a living cell) in a sample can be detected colorimetrically, fluorimetrically, or lumimetrically.

Detection systems are known in the art and can be used to detect biological analytes colorimetrically (i.e., by the absorbance and/or scattering of light), fluorescently, or lumimetrically. Examples of the detection of biomolecules by luminescence are described by F. Gorus and E. Schram (Applications of bio- and chemiluminescence in the clinical laboratory, 1979, Clin. Chem. 25:512-519).

An example of a biological analyte detection system is an ATP detection system. The ATP detection system can comprise an enzyme (e.g., luciferase) and an enzyme substrate (e.g., luciferin). The ATP detection system can further comprise a luminometer. In some embodiments, the luminometer can comprise a bench top luminometer, such as the FB-12 single tube luminometer (Berthold Detection Systems USA, Oak Ridge, Tenn.). In some embodiments, the luminometer can comprise a handheld luminometer, such as the NG Luminometer, UNG2 (3M Company, Bridgend, U.K.).

Methods of the present disclosure include the formation of a liquid mixture comprising a sample suspected of containing live cells and a release element. Methods of the present disclosure further include detecting a biological analyte. Detecting a biological analyte can further comprise quantitating the amount of biological analyte in the sample.

In some embodiments, detecting the biological analyte can comprise detecting the analyte directly in a vessel (e.g., a tube, a multi-well plate, and the like) in which the liquid mixture comprising the sample and the release element is formed. In some embodiments, detecting the biological analyte can comprise transferring at least a portion of the liquid mixture to a container other than the vessel in which the liquid mixture comprising the sample and the release element is formed. In some embodiments, detecting the biological analyte may comprise one or more sample preparation processes, such as pH adjustment, dilution, filtration, centrifugation, extraction, and the like. In some embodiments, detecting the analyte can comprise detecting the analyte genetically or immunologically.

In some embodiments, the biological analyte is detected at a single time point. In some embodiments, the biological analyte is detected at two or more time points. When the biological analyte is detected at two or more time points, the amount of biological analyte detected at a first time (e.g., before an effective amount of cell extractant is released from a release element to effect the release of biological analytes from live cells in at least a portion of the sample) point can be compared to the amount of biological analyte detected at a second time point (e.g., after an effective amount of cell extractant is released from a release element to effect the release of biological analytes from live cells in at least a portion of the sample). In some embodiments, the measurement of the biological analyte at one or more time points is performed by an instrument with a processor. In certain preferred embodiments, comparing the amount of biological analyte at a first time point with the amount of biological analyte at a second time point is performed by the processor.

For example, the operator measures the amount of biological analyte in the sample after the liquid mixture including the sample and the release element is formed. The amount of biological analyte in this first measurement (T₀) can indicate the presence of “free” (i.e. acellular) biological analyte and/or biological analyte from nonviable cells in the sample. In some embodiments, the first measurement can be made immediately (e.g., about 1 second) after the liquid mixture including the sample and the release element is formed. In some embodiments, the first measurement can be at least about 5 seconds, at least about 10 seconds, at least about 20 seconds, at least about 30 seconds, at least about 40 seconds, at least about 60 seconds, at least about 80 seconds, at least about 100 seconds, at least about 120 seconds, at least about 150 seconds, at least about 180 seconds, at least about 240 seconds, at least about 5 minutes, at least about 10 minutes, at least about 20 minutes after the liquid mixture including the sample and the release element is formed. These times are exemplary and include only the time up to that the detection of a biological analyte is initiated. Initiating the detection of a biological analyte may include diluting the sample and/or adding a reagent to inhibit the activity of the cell extractant. It will be recognized that certain detection systems (e.g., nucleic acid amplification or ELISA) can generally take several minutes to several hours to complete.

The operator allows the sample to contact the release element comprising the cell extractant for a period of time after the first measurement of biological analyte has been made. After the sample has contacted the release element for a period of time, a second measurement of the biological analyte is made. In some embodiments, the second measurement can be made up to about 0.5 seconds, up to about 1 second, up to about 5 seconds, up to about 10 seconds, up to about 20 seconds, up to about 30 seconds, up to about 40 seconds, up to about 60 seconds, up to about 90 seconds, up to about 120 seconds, up to about 180 seconds, about 300 seconds, at least about 10 minutes, at least about 20 minutes, at least about 60 minutes or longer after the first measurement of the biological analyte. These times are exemplary and include only the interval of time from which the first measurement for detecting the biological analyte is initiated and the time at which the second measurement for detecting the biological analyte is initiated. Initiating the detection of a biological analyte may include diluting the sample and/or adding a reagent to inhibit the activity of the cell extractant.

Preferably, the first measurement of a biological analyte is made about 1 second to about 240 seconds after the liquid mixture including the sample and the release element is formed and the second measurement, which is made after the first measurement, is made about 1.5 seconds to about 540 seconds after the liquid mixture is formed. More preferably, the first measurement of a biological analyte is made about 1 second to about 180 seconds after the liquid mixture is formed and the second measurement, which is made after the first measurement, is made about 1.5 seconds to about 120 seconds after the liquid mixture is formed. Most preferably, the first measurement of a biological analyte is made about 1 second to about 5 seconds after the liquid mixture is formed and the second measurement, which is made after the first measurement, is made about 1.5seconds to about 10 seconds after the liquid mixture is formed.

The operator compares the amount of a biological analyte detected in the first measurement to the amount of biological analyte detected in the second measurement. An increase in the amount of biological analyte detected in the second measurement is indicative of the presence of one or more live cells in the sample.

In certain methods, it may be desirable to detect the presence of live somatic cells (e.g., nonmicrobial cells). In these embodiments, the release element comprises a cell extractant that selectively releases biological analytes from somatic cells. Nonlimiting examples of somatic cell extractants include nonionic detergents, such as non-ionic ethoxylated alkylphenols, including but not limited to the ethoxylated octylphenol Triton X-100 (TX-100) and other ethoxylated alkylphenols; betaine detergents, such as carboxypropylbetaine (CB-18), NP-40, TWEEN, Tergitol, Igepal, commercially available M-NRS (Celsis, Chicago, Ill.), M-PER (Pierce, Rockford, Ill.), CelLytic M (Sigma Aldrich). Cell extractants are preferably chosen not to inactivate the analyte and its detection reagents.

In certain methods, it may be desirable to detect the presence of live microbial cells. In these embodiments, the release element can comprise a cell extractant that selectively releases biological analytes from microbial cells. Nonlimiting examples of microbial cell extractants include quaternary ammonium compounds, including benzalkonium chloride, benzethonium chloride, ‘cetrimide’ (a mixture of dodecyl-, tetradecyl- and hexadecyl-trimethylammoium bromide), cetylpyridium chloride; amines, such as triethylamine (TEA) and triethanolamine (TeolA); bis-Biguanides, including chlorhexidine, alexidine and polyhexamethylene biguanide Dialkyl ammonium salts, including N-(n-dodecyl)-diethanolamine, antibiotics, such as polymyxin B (e.g., polymyxin B1 and polymyxin B2), polymyxin-beta-nonapeptide (PMBN); alkylglucoside or alkylthioglucoside, such as Octyl-β-D-1-thioglucopyranoside (see U.S. Pat. No. 6,174,704 herein incorporated by reference in its entirety); nonionic detergents, such as non-ionic ethoxylated alkylphenols, including but not limited to the ethoxylated octylphenol Triton X-100 (TX-100) and other ethoxylated alkylphenols; betaine detergents, such as carboxypropylbetaine (CB-18); and cationic, antibacterial, pore forming, membrane-active, and/or cell wall-active polymers, such as polylysine, nisin, magainin, melittin, phopholipase A₂, phospholipase A₂ activating peptide (PLAP); bacteriophage; and the like. See e.g., Morbe et al., Microbiol. Res. (1997) vol. 152, pp. 385-394, and U.S. Pat. No. 4,303,752 disclosing ionic surface active compounds which are incorporated herein by reference in their entirety. Cell extractants are preferably chosen not to inactivate the biological analyte and/or a detection reagent used to detect the biological analyte.

In certain alternative methods to detect the presence of live microbial cells in a sample, the sample can be pretreated with a somatic cell extractant for a period of time (e.g., the sample is contacted with a somatic cell extractant for a sufficient period of time to extract somatic cells before a liquid mixture including the sample and a release element comprising a microbial cell extractant is formed). In the alternative embodiment, the amount of biological analyte detected at the first measurement will include any biological analyte that was released by the somatic cells and the amount of additional biological analyte, if any, detected in the second measurement will include biological analyte from live microbial cells in the sample.

### EXAMPLES

The present invention has now been described with reference to several specific embodiments foreseen by the inventor for which enabling descriptions are available. Insubstantial modifications of the invention, including modifications not presently foreseen, may nonetheless constitute equivalents thereto. Thus, the scope of the present invention should not be limited by the details and structures described herein, but rather solely by the following claims, and equivalents thereto.

### Preparative Example 1

### Incorporation of Cell Extractant into Hydrogel Beads During Polymerization of the Hydrogel

Beads were made as described in example 1 of International Patent Publication No. WO 2007/146722, in which the deionized water was replaced with the desired loading solution. A homogeneous precursor composition was prepared by mixing 40 grams of 20-mole ethoxylated trimethylolpropane triacrylate (EO₂₀-TMPTA) (SR415 from Sartomer, Exeter, Pa.), 60 grams deionized (DI) water, and 0.8 grams photoinitiator (IRGACURE 2959 from Ciba Specialty Chemicals, Tarrytown, N.Y.). The precursor composition was poured into a funnel such that the precursor composition exited the funnel through a 2.0 millimeter diameter orifice. Precursor composition fell along the vertical axis of a 0.91 meter long, 51 millimeter diameter quartz tube that extended through a UV exposure zone defined by a light shield and a 240 W/cm irradiator (available from Fusion UV Systems, Gaithersburg, Md.) equipped with a 25-cm long “H” bulb coupled to an integrated back reflector such that the bulb orientation was parallel to falling precursor composition. Below the irradiator, polymeric beads were obtained. The entire process was operated under ambient conditions

The BARDAC 205 M and 208M (blends of quaternary ammonium compounds and alkyl dimethyl benzyl ammonium chloride; Lonza Group Ltd., Valais, Switzerland) hydrogel beads were prepared by mixing 20 grams of EO₂₀-TMPTA, 30 grams of the BARDAC 205M or 208M solution and 0.4 grams of Irgacure 2959 and exposed to UV light to prepare beads as described in example 1 in International Patent Publication No. WO 2007/146722. The beads were prepared using 12.5% and 25% (w/v) solutions of BARDAC 205M and 208M in deionized water. After recovering the beads, they were stored in a jar at room temperature. The beads were designated as shown below:

| | |
|---|---|
| 25% 205M solution bead | 205M-1s |
| 12.5% 205M solution bead | 205M-2s |
| 25% 208M solution bead | 208M-1s |
| 12.5% 208M solution bead | 208M-2s |

### Preparative Example 2

### Incorporation of Cationic Monomers into Hydrogel Beads During Polymerization of the Hydrogel

Polymeric beads with cationic monomers were prepared as described in Example 30 to 34 of International patent WO2007/146722. The precursor composition used for making beads is indicated in Table 1. The various components of the precursor compositions were stirred together in an amber jar until the antimicrobial monomer dissolved.

DMAEMA-C₈Br was formed within three-neck round bottom reaction flask that was fitted with a mechanical stirrer, temperature probe and a condenser. The reaction flask was charged with 234 parts of dimethylaminoethylmethacrylate, 617 part of acetone, 500 parts 1-bromoethane, and 0.5 parts of BHT antioxidant. The mixture was stirred for 24 hours at 35° C. At this point, the reaction mixture was cooled to room temperature and a slightly yellow clear solution was obtained. The solution was transferred to a round bottom flask and acetone was removed by rotary evaporation under vacuum at 40° C. The resulting solids were washed with cold ethyl acetate and dried under vacuum at 40° C. DMAEMA-C₁₀Br and DMAEMA-C₁₂Br were formed using a similar procedure in which the 1-bromooctane was replaced by 1-bromodecane and 1-bromododecane, respectively.

The 3-(acryloamidopropyl)trimethylammonium chloride was obtained by Tokyo Kasei Kogyo Ltd (Japan). Ageflex FA-1Q80MC was obtained from Ciba Specialty Chemicals.

TABLE 1
Beads with antimicrobial Monomer

| | | Antimi- | | | |
|---|---|---|---|---|---|
| | | crobial | | | |
| | | mon- | Propylene | | Irgacure |
| Bead | Cationic monomer | omer | Glycol | SR415 | 2959 |
| C₈-1s | DMAEMA-C₈Br | 1.86 g | 7.44 g | 13.02 g | 0.30 g |
| C₁₀-1s | DMAEMA-C₁₀Br | 1.91 g | 7.60 g | 13.30 g | 0.30 g |
| C₁₂-1s | DMAEMA-C₁₂Br | 1.92 g | 7.68 g | 13.44 g | 0.31 g |
| ATAC- | 3- | 2.34 g | 9.38 g | 17.50 g | 0.40 g |
| 1s | (acryloamidopropyl) | | | | |
| | trimethylammonium | | | | |
| | chloride | | | | |
| Ageflex- | Ageflex FA- | 2.50 g | 10.00 g | 17.50 g | 0.40 g |
| 1s | 1Q80MC | | | | |

### Preparative Example 3

### Incorporation of Luciferin into Hydrogel Beads During Polymerization of the Hydrogel

Hydrogel beads containing luciferin were made similarly by mixing 20 parts of EO₂₀-TMPTA with 30 parts of luciferin (2 mg in 30 ml of 14 mM of phosphate buffer, pH 6.4) and 0.4 parts photoinitiator (IRGACURE 2959) and exposed to UV light to prepare beads as described in example 1 in International Patent Publication No. WO 2007/146722 A1. The beads were then stored in a jar at 4° C. and designated as Luciferin-1s.

### Preparative Example 4

### Incorporation of Luciferase into Hydrogel Beads During Polymerization of the Hydrogel

Hydrogel beads containing luciferase were made by mixing 20 parts of polymer with 30 parts of luciferase (150 μl of 6.8 mg/ml in 30 ml of 14 mM of phosphate buffer, pH 6.4) and 0.4 parts photoinitiator (IRGACURE 2959) and exposed to UV light to prepare beads as described in example 1 in International Patent Publication No. WO 2007/146722 A1. The beads were then stored in a jar at 4° C. and designated as Luciferase-1s.

### Preparative Example 5

### Incorporation of Cell Extractant into Hydrogel Beads After Polymerization of the Hydrogel

Hydrogel beads were prepared as described in example 1 International Patent Publication No. WO 2007/146722. Active beads were prepared by drying as described in example 19 and then soaking in active solution as described in example 23 of International Patent Publication No. WO 2007/146722. One gram of beads was dried at 60° C. for 2 h to remove water from the beads. The dried beads were dipped in 2 grams of BARDAC 205M for at least 3 hrs to overnight at room temperature. After soaking, the beads were poured into a Buchner funnel to drain the beads and then rinsed with 10 to 20 ml of distilled water. The excess water was removed from the surface of the beads by blotting them with a paper towel. The beads were prepared using 10%, 12.5%, 20%, 25%, 50% and 100% (w/v) aqueous solutions of BARDAC 205M, 5%,10%,12.5%, 25% and 50% solutions of 208M, 20% solution of Triclosan (Ciba Specialty Chemicals,), 1% and 5% solutions of chlorohexidine digluconate (CHG; Sigma Aldrich, St. Louis, Mo.) and 0.25% and 0.5% solutions of Cetyltrimethylammoniumbromide (CTAB; Sigma Aldrich). The beads were then stored in a jar at room temperature. The beads were designated as shown below.

| | |
|---|---|
| 100% 205M solution bead | 205M-1p |
| 50% 205M solution bead | 205M-2p |
| 25% 205M solution bead | 205M-3p |
| 20% 205M solution bead | 205M-4p |
| 12.5% 205M solution bead | 205M-5p |
| 10% 205M solution bead | 205M-6p |
| 50% 208M solution bead | 208M-1p |
| 25% 208M solution bead | 208M-2p |
| 12.5% 208M solution bead | 208M-3p |
| 10% 208M solution bead | 208M-4p |
| 5% 208M solution bead | 208M-5 |
| 20% Triclosan solution bead | Triclosan-1p |
| 1% CHG solution bead | CHG-1p |
| 5% CHG solution bead | CHG-2p |
| 0.25% CTAB solution bead | CTAB-1p |
| 0.5% CTAB solution bead | CTAB-2p |

Hydrogel beads of VANTOCIL (Arch Chemicals, Norwalk, Conn.), CARBOSHIELD (Lonza) and a blend of VANTOCIL and CARBOSHIELD were prepared similarly. The dried hydrogel beads were dipped in 50% solution (in distilled water) of VANTOCIL or 100% solution of CARBOSHIELD 1000 or 1:1 mixture of 50% VANTOCIL and 100% CARBOSHIELD solutions. The beads with the mixture of VANTOCIL and CARBOSHIELD resulted in 25% VANTOCIL and 50% CARBOSHIELD beads. The beads were then stored in a jar at room temperature and designated as follows

| | |
|---|---|
| 50% VANTOCIL solution bead | Van-1p |
| 100% CARBOSHIELD solution bead | Carbo-1p |
| 25% VANTOCIL and 50% CARBOSHIELD solution bead | Van- |
| | Carbo-1p |

### Preparative Example 6

### Incorporation of Cell Extractant into Hydrogel Fibers After Polymerization of the Hydrogel

Polymeric fibers were made as described in example 1 of US Patent Application Publication No. US2008/207794. A homogeneous precursor composition was prepared that contained about 500 grams of 40 wt-% 20-mole EO₂₀-TMPTA (SR415 from Sartomer) and 1 wt-% photoinitiator (IRGACURE 2959 from Ciba Specialty Chemicals) in deionized water. The precursor composition was processed as described in example 1 of US Patent Application Publication No. US2008/207794 to make the polymeric fibers.

One gram of fibers was dried at 60° C. for 2 h to remove water from the fibers. The dried fibers were dipped in 2 grams of 50% solution of BARDAC 205M for at least 3 hrs to overnight at room temperature. After soaking, the fibers were poured into a Buchner funnel to drain the fibers and then rinsed with 10 to 20 ml of distilled water. The excess water was removed from the surface of the fibers by blotting them with a paper towel. The fibers were then stored in a jar at room temperature.

### Preparative Example 7

### Incorporation of Luciferin into Hydrogel Beads After Polymerization of the Hydrogel

Hydrogel beads (1× gram) were dried at 60° C. for 2 h and dipped in 2× grams of luciferin solution (2 mg in 30 ml of 14 mM of phosphate buffer, pH6.4) for at least 16 h at 4° C. After soaking, the beads were poured into a Buchner funnel to drain the beads and then rinsed with distilled water. The excess water was removed from the surface of the beads by blotting them with a paper towel. The beads were then stored in a jar at 4° C. and designated as Lucifein-1p

### Preparative Example 8

### Incorporation of Enzymes into Hydrogel Beads After Polymerization of the Hydrogel

Hydrogel beads (1× gram) were dried at 60° C. for 2 h and dipped in 2× grams of luciferase solution (150 μl of 6.8 mg/ml luciferase in 30 ml of 14 mM of phosphate buffer, pH6.4) for at least 16 h at 4° C. After soaking, the beads were poured into a Buchner funnel to drain the beads and then rinsed with distilled water. The excess water was removed from the surface of the beads by blotting them with a paper towel. Hydrogel beads containing lysozyme or lysostaphin were prepared similarly by soaking in 2× grams of 50 mM TRIS pH 8.0 solution containing 0.5 mg/ml lysozyme or 50 μg/ml lysostaphin. The beads were then stored in a jar at 4° C. and designated as Luciferase-1p, Lysozyme-1p and Lysostaphin-1p.

### Preparative Example 9

### Preparation of Microtablets Containing Cell Extractants

Microtablets were formed from a mixture containing lysis reagent (Benzalkonium chloride, Alfa Aesar, Ward Hill, Mass.; cetyltrimethylammonium bromide, CTAB, Sigma-Aldrich. St. Louis, Mo.; chlorohexidine dihydrochloride, MP Biomedicals, Solon, Ohio; or chlorohexidine diacetate, MP Biomedicals), mannitol (Sigma-Aldrich), L-leucine (Nutrabio.com. Middlesex, N.J.) and CAB-O-SIL® TS-530 (Cabot Corporation, Billerica, Mass.) (Table 2) using a hand operated Arbor Press. The mannitol acts as a diluent/binder and its slow dissolution rate helps in sustained release of the active. Cab-O-Sil is a glidant/anti-caking agent that enhances the flow of a granular mixture by reducing interparticle friction. L-Leucine is a water-soluble lubricant and an anti-adherent that prevents binding of the microtabletting powder to the press.

TABLE 2
Reagent mixture for microtabletting

| | 0.025% active | 0.05% active |
|---|---|---|
| Reagents | microtablet | microtablet |
| Lysis Reagent | 50 mg | 100 mg |
| Cab-O-Sil ® TS-530 | 5 mg | 5 mg |
| L-Leucine | 100 mg | 100 mg |
| Mannitol | 1845 mg | 1795 mg |

The reagents except leucine were weighed out and added to a 50-ml ball-mill tube and the ball-mill tube was placed in dry ice for 20 minutes. The reagents were ball-milled for 2×40 seconds at 16^{-s} frequency and added to a glass scintillation vial. The mixture was then vortexed on a Vortex-Genie (Fisher Scientific, Bohemia, N.Y.) for 2 minutes. L-Leucine (jet-milled to <10 μm) was weighed out and added to the vial and vortexed for 2 minutes to provide a well mixed powder exhibiting substantially uniform distribution of the reagents. The resulting mixture was formed into microtablets using a single leverage lab Arbor Press (Dake, Grand Haven, Mich.) fitted with a custom made 2 mm diameter stainless steel punch and die set equipped with spacers for adjusting fill volume. Control microtablets were made similarly with a mixture containing mannitol, leucine and Cab-O-Sil but no lysis reagent.

The Arbor Press was operated using an electronic torque wrench (Model #7767A12 from Mc-Master Can, Atlanta, Ga.). The fill volume was adjusted to obtain a compressed microtablet weight of 2 milligrams. Each microtablet contained either 0.025% or 0.05% of the desired lysis reagent. The microtablets were compressed at a pressure of 155 MPa.

### Preparative Example 10

### Preparation of Microtablets Containing Luciferase and Luciferin

Microtablets were formed from a mixture containing luciferase and luciferin, sorbitol (Sigma-Aldrich), leucine and Cab-O-Sil (Table 3) using a hand operated Arbor Press. Twenty ml of UltraGlo luciferase (9 mg/lit, Promega, Madison, Wis.) and luciferin (0.05 mg/lit, Promega)) in 16 mM ADA (N-(2-Acetamido) Iminodiacetic Acid; N-(Carbamoylmethyl)Iminodiacetic Acid) buffer and 20 ml of luciferase (7.8 mg/lit, 3M Bridgend, UK) and luciferin (5.5 mg/lit, Promega) in 14 mM in Phosphate buffer were lyophilized.

TABLE 3
Reagent mixture for enzyme microtabletting

| Reagents | Luciferase-Luciferin | UltraGlo luciferase-Luciferin |
|---|---|---|
| Luciferase/Luciferin | 3.98 g | 1.8 g |
| Cab-O-Sil ® TS- | 13.5 mg | 8.2 mg |
| 530 | | |
| Leucine | 0.27 g | 0.164 g |
| Sorbitol | 1.16 g | 1.30 g |

The lyophilized enzyme mixture was placed in a mortar and ground with a pestle and added to a scintillation vial. Pre-ball milled sorbitol (sieved to <300 μm) was added to the glass scintillation vial and the formulation was vortexed for 2 minutes. Later Cab-O-Sil was added and vortexed for 2 minutes. L-Leucine (jet-milled to <10 μm) was weighed out and added to the vial and vortexed for 2 minutes to provide a well mixed powder exhibiting substantially uniform distribution of the reagents. The resulting mixture was formed into microtablets using a single leverage lab Arbor Press fitted with a custom made 3 mm diameter stainless steel punch and die set equipped with spacers for adjusting fill volume. The Arbor Press was operated using an electronic torque wrench. The fill volume was adjusted to obtain a compressed microtablet weight of 20 or 30 milligrams. The microtablets were compressed at a pressure of 155 MPa.

### Preparative Example 11

### Preparation of Films Containing Extractants

A 3% polyvinyl alcohol solution (1.5% of PVOH-26-88 and 1.5% of PVOH 403) was prepared in deionized water and the solution was agitated on a shaker in a warm bath for 24 hours to allow the PVOH to fully dissolve. An antimicrobial film forming solution containing 0.5% carboquat, 0.5% VANTOCIL and 0.5% GLUCOPON 425N was made in the 3% PVOH solution. Polyethylene terephthalate (PET) films were coated with the antimicrobial solution using a Meyer rod #6. The coating was allowed to dry on the substrate at room temperature and the dried films were stored at room temperature. The coated film, dried film was die-cut into circular disks having approximately 7 mm diameter. Negative controls disks were prepared by coating PET film with a 3% PVOH solution containing no cell extractant, drying the coated film, and die-cutting the coated film into 7 mm disks.

### Preparative Example 12

### Preparation of Various Matrices Containing Extractants

Various matrices were dipped in the extractant solution containing polyvinyl alcohol, VANTOCIL and CARBOSHIELD or 5% benzalkonium chloride solution (Alfa Aesar). The matrices were removed and dried at 70° C. for about an hour and stored at room temperature. The matrices used were Grade 54, 22 μm Quantitative Filter Paper, Grade 4, 20-25 μm Qualitative Filter Paper, Grade 30, Glass-Fiber Filter Paper, Grade GB005, a thick (1.5 mm) highly absorbent blotting paper (all obtained from Whatman, Inc, Florham Park, N.J.), Zeta Plus Virosorb 1MDS discs (CUNO, Inc, Meriden, Conn.) and 0.45 μm MF-Millipore membrane (Millipore, Billerica, Mass.)

### Example 1

### Effect of BARDAC 205M Disinfectant-Loaded Hydrogel Beads on the Release of ATP from S. aureus and E. coli Cells

The microbial species used in the examples (Table 4) were obtained from ATCC (Manassas, Va.). 3M™ Clean-Trace™ Surface ATP system and NG Luminometer UNG2 were obtained from 3M Company (St. Paul, Minn.). Rayon-tipped applicators were obtained from Puritan Medical Products (Guilford, Me.). Beads containing BARDAC 205M were made according to Preparative Example 5.

TABLE 4
Microorganisms used in examples

| Microorganism | ATCC No. |
|---|---|
| Candida albicans | MYA-2876 |
| Candida albicans | 10231 |
| Corynebacterium xerosis | 373 |
| Enterococcus faecalis | 49332 |
| Enterococcus faecalis | 700802 |
| Enterococcus faecium | 6569 |
| Enterococcus faecium | 700221 |
| Escherichia coli | 51183 |
| Kocuria kristinae | BAA-752 |
| Micrococcus luteus | 540 |
| Pseudomonas aeruginosa | 9027 |
| Salmonella enterica subsp. enterica | 4931 |
| Staphylococcus aureus | 6538 |
| Staphylococcus epidermidis | 14990 |
| Streptococcus pneumoniae | 6301 |

Pure cultures of the bacterial strains were inoculated into tryptic soy broth and were grown overnight at 37° C. Swabs from some of the Clean-Trace surface ATP hygiene tests, which include microbial cell extractants, were replaced with sterile rayon-tipped applicators, which do not include microbial cell extractants. Various amounts (approximately 10⁶, 10⁷ and 10⁸, colony-forming units (CFU) per milliliter, respectively) of bacteria were suspended in Butterfield's buffer and cell suspensions were added directly to the Clean-Trace surface ATP swabs (10 microliters) or the rayon-tipped applicators (100 microliters). Each swab or applicator was activated by pushing it into the reagent chamber according to the manufacturer's instructions. The test unit was immediately inserted into the reading chamber of a NG Luminometer, UNG2 and an initial (T₀) measurement of Relative Light Units (RLUs) was recorded. One BARDAC 205M-containing hydrogel bead, 205M-1p, was added to some of the test units and subsequent RLU measurements were recorded at 20 sec interval using the “Unplanned Testing” mode of the luminometer until the number of RLUs reached a plateau. The data were downloaded using the software provided with the NG luminometer. 205M-1p beads were able to lyse bacteria and release ATP from cells, as shown by the data in Table 5. The relative light units (RLU) increased over time with BARDAC 205M beads, while without beads the background did not increase. Experiments using the Clean-Trace surface ATP swabs showed that the RLU reached maximum within 20 seconds and then began to decrease.

TABLE 5
Detection of ATP from microbial cells exposed to microbial cell extractants released from hydrogels.

| | S. aureus | | | | | | E. coli | | | | | |
|---|---|---|---|---|---|---|---|---|---|---|---|---|
| | 10⁵ CFU | | | 10⁶ CFU | | | 10⁵ CFU | | | 10⁶ CFU | | |
| Time | RA | RA | CT | RA | RA | CT | RA | RA | CT | RA | RA | CT |
| (sec) | 0 bead | 1 bead | 0 bead | 0 bead | 1 bead | 0 bead | 0 bead | 1 bead | 0 bead | 0 bead | 1 bead | 0 bead |
| 0 | 64 | 226 | 1175 | 1183 | 1647 | 8140 | 28 | 308 | 1235 | 228 | 338 | 7557 |
| 20 | 71 | 236 | 1183 | 1161 | 1709 | 8215 | 29 | 310 | 1243 | 230 | 345 | 7684 |
| 40 | 84 | 288 | 1185 | 1175 | 2042 | 8262 | 30 | 317 | 1250 | 243 | 656 | 7764 |
| 80 | 92 | 301 | 1166 | 1179 | 2158 | 8053 | 31 | 326 | 1251 | 245 | 763 | 7772 |
| 120 | NR | 334 | NR | NR | 2237 | NR | 30 | 343 | 1249 | 244 | 973 | 7781 |
| 160 | NR | 463 | NR | NR | 2955 | NR | 28 | 353 | NR | 246 | 1463 | 7504 |
| 200 | NR | 643 | NR | NR | 5612 | NR | 31 | 428 | NR | 243 | 2036 | NR |
| 240 | NR | 776 | NR | NR | 6807 | NR | NR | 531 | NR | NR | 2570 | NR |
| 280 | NR | 852 | NR | NR | 6919 | NR | NR | 629 | NR | NR | 3614 | NR |
| 320 | NR | 899 | NR | NR | 7050 | NR | NR | 639 | NR | NR | 4687 | NR |
| 360 | NR | 963 | NR | NR | 7303 | NR | NR | 633 | NR | NR | 5078 | NR |
| 400 | NR | 996 | NR | NR | 7345 | NR | NR | NR | NR | NR | 5288 | NR |

Values expressed in the table are relative light units (RLUs).
RA = rayon-tipped applicator,
CT = Clean-Trace surface ATP swab,
NR = not recorded.
BARDAC 205M beads, 205M-1p if present, were added to the sample immediately after the T₀ measurement was obtained.

### Example 2

### Effect of VANTOCIL and CARBOSHIELD Disinfectant-Loaded Hydrogel Beads on the Release of ATP from S. aureus

A S. aureus overnight culture was prepared as described in Example 1. Hydrogel beads containing VANTOCIL and/or CARBOSHIELD were prepared as described in Preparative Example 5. The luciferase/luciferin liquid reagent solution (300 μl) was removed from Clean-Trace surface ATP hygiene test units and transferred to 1.5 ml microfuge tubes. The bacterial culture was diluted to 10⁷ CFU/ml in Butterfield's buffer and 10 microliters of the diluted suspension were added directly to individual microfuge tubes (i.e., approximately 10⁵ CFU per tube). Immediately after adding the bacterial suspension, the tube was placed into a bench-top luminometer (FB-12 single tube luminometer, Berthold Detection Systems USA, Oak Ridge, Tenn.) and an initial (T₀) measurement of RLUs was recorded. The initial (and all subsequent luminescence measurements) were obtained from the luminometer using FB12 Sirius PC software that was provided with the luminometer. The light signal was integrated for 1 second and the results are expressed in RLU/sec.

A hydrogel bead containing VANTOCIL (Van-1p), CARBOSHIELD (Carbo-1p), or both VANTOCIL and CARBOSHIELD (Van-Carbo-1p) was added to individual tubes and RLU measurements were recorded at 10 sec intervals until the number of RLUs reached a plateau (Table 3).

The hydrogel beads, containing individual disinfectants or a disinfectant mixture, extracted ATP from the S. aureus cells and the ATP reacted with the ATP-detection reagents of the Clean-Trace surface ATP units, as shown in Table 6. The relative light units (RLU) increased over time in the tubes that received the disinfectant-loaded beads, while the tubes without beads did not show a significant increase in RLU over time.

TABLE 6
Detection of ATP released from S. aureus cells after exposure to
VANTOCIL- and/or CARBOSHIELD-loaded hydrogel beads.

| | | | | VANTOCIL + |
|---|---|---|---|---|
| | | VANTOCIL | CARBOSHIELD | CARBOSHIELD |
| Time | | Bead | Bead | Bead |
| (sec) | No Bead | (Van-1p) | (Carbo-1p) | (Van-Carbo-1p) |
| 0 | 840 | 994 | 1354 | 5150 |
| 50 | 910 | 2809 | 2745 | 5202 |
| 100 | 940 | 5529 | 6868 | 6228 |
| 200 | 950 | 9246 | 12292 | 9243 |
| 300 | 920 | 13413 | 15110 | 14341 |
| 400 | 910 | 19723 | 17107 | 19337 |
| 600 | 780 | 35195 | 22725 | 29997 |
| 800 | NR | 50421 | 28719 | 38939 |
| 1000 | NR | 59389 | 32822 | 46965 |
| 1200 | NR | 59872 | 33252 | 51271 |
| 1600 | NR | 56717 | 33401 | 60293 |
| 1800 | NR | 52527 | 31483 | 63154 |

NR = not recorded.
Beads containing extractants, if present, were added to the sample immediately after the T₀ measurement was obtained.

### Example 3

### Effect of the Number of Disinfectant-Loaded Beads on the Release of ATP from S. aureus and E. coli Cells

S. aureus and E. coli overnight cultures were prepared as described in Example 1. 3M Clean-Trace surface ATP system swabs were replaced with sterile rayon-tipped applicators, as described in Example 1. The bacterial suspensions were diluted to approximately 10⁷ CFU/ml in Butterfield's buffer. One hundred-microliter aliquots of the suspension were added directly to the swabs. BARDAC 205M hydrogel beads were prepared as described in Preparative Example 5. Up to three hydrogel beads (i.e., 0 bead, 1 bead, or 3 beads) were added to individual test units and each applicator was inserted into a Clean-Trace surface ATP test unit to activate ATP detection according to the manufacturer's instructions. The test unit was immediately inserted into the reading chamber of a NG Luminometer, UNG2 and RLU measurements were recorded at 20 sec intervals using the “Unplanned Testing” mode of the luminometer until the number of RLUs reached a plateau. The results are shown in Table 7. The data indicate that the BARDAC 205M beads, 205M-1p, permeabilized the bacteria, causing release of ATP from cells. The relative light units (RLU) increased over time in the samples containing the BARDAC beads, with a larger increase observed in a short period of time with higher number of beads. In contrast, the samples without the beads did not show a similar increase in RLU.

TABLE 7
Detection of ATP from microbial cells exposed to various
amounts of BARDAC 205M hydrogel beads. BARDAC
205M beads, 205M-1p, if present, were added to the sample
immediately before the first measurement was obtained.

| | S. aureus | | | E. coli | | |
|---|---|---|---|---|---|---|
| Time (sec) | 0 bead | 1 bead | 3 beads | 0 bead | 1 bead | 3 beads |
| 10 | 1066 | 1647 | 3143 | 837 | 338 | 1651 |
| 20 | 1051 | 1709 | 4574 | 892 | 345 | 2031 |
| 40 | 1058 | 2042 | 5885 | 940 | 656 | 2524 |
| 80 | 1055 | 2158 | 6836 | 962 | 763 | 2956 |
| 120 | 1063 | 2237 | 7509 | 965 | 973 | 3368 |
| 160 | 1047 | 2955 | 8230 | 1020 | 1463 | 4263 |
| 200 | 1048 | 5612 | 8610 | 1052 | 2036 | 5048 |
| 240 | 1051 | 6807 | 8851 | 1067 | 2570 | 5695 |
| 280 | 1043 | 6919 | 8993 | 1090 | 3614 | 6232 |
| 320 | 1039 | 7050 | 9117 | 1091 | 4687 | 6682 |
| 360 | 1033 | 7303 | 9164 | 1127 | 5078 | 6975 |
| 400 | 1025 | 7345 | 9171 | 1127 | 5288 | 7266 |

### Example 4

### Detection of ATP from Microbial Cells Exposed to Various Amounts of a Microbial Cell Extractant

S. aureus and E. coli overnight cultures were prepared as described in Example 1. Immediately before use in these tests, the bacterial suspensions were diluted in Butterfield's buffer to concentrations of approximately 10⁶ and 10⁷ CFU per milliliter. Luciferase/luciferin reagent (300 μl) from Clean-Trace surface ATP system was removed and added to 1.5 ml microfuge tubes. Ten-microliter amounts of the bacterial suspensions were added directly to individual microfuge tubes containing the reagents. BARDAC 205M hydrogel beads were prepared as described in Preparative Example 5. Up to three hydrogel beads (i.e., 0 beads, 1 bead, 2 beads or 3 beads) were added to each tube. Relative Light Units (RLUs) were recorded at 10 sec interval in a bench top luminometer (FB-12 single tube luminometer with software), as described in Example 2. The results of the experiments are shown in Table 8. The results indicate that the BARDAC 205M beads, 205M-1p, were able to lyse bacteria and release ATP from cells. The relative light units (RLU) increased over time in tubes containing at least one BARDAC 205M bead, with a larger increase observed in a short period of time with higher number of beads. Tubes containing no beads did not show a significant increase in RLU's.

TABLE 8
Detection of ATP from microbial cells exposed to various amounts of BARDAC 205M hydrogel beads.

| | S. aureus | | | | | E. coli | | | | | | | | |
|---|---|---|---|---|---|---|---|---|---|---|---|---|---|---|
| | 10⁵ CFU | | | | | 10⁵ CFU | | | | | 10⁶ CFU | | | |
| Time | RA | RA | RA | RA | CT | RA | RA | RA | RA | CT | RA | RA | RA | RA |
| (sec) | 0 bead | 1 bead | 2 beads | 3 beads | 0 bead | 0 bead | 1 bead | 2 beads | 3 beads | 0 bead | 0 bead | 1 beads | 2 beads | 3 beads |
| 10 | 470 | 1770 | 2147 | 1888 | 21489 | 1371 | 3208 | 5537 | 8996 | 41489 | 1820 | 6646 | 12765 | 18981 |
| 20 | 500 | 2500 | 2528 | 4185 | 35610 | 1486 | 3330 | 11498 | 38219 | 45610 | 1865 | 9682 | 24253 | 136641 |
| 40 | 55 | 3315 | 4894 | 26452 | 50678 | 1495 | 5716 | 46091 | 60362 | 53111 | 1920 | 12470 | 69865 | 172179 |
| 80 | 571 | 5771 | 17148 | 41192 | 55568 | 1502 | 46047 | 53283 | 59372 | 55412 | 1980 | 30875 | 146756 | 179238 |
| 120 | 608 | 19088 | 32480 | 51329 | 48785 | 1500 | 51490 | 52499 | 59344 | 49655 | 1940 | 85141 | 187122 | 170591 |
| 160 | 596 | 39596 | 42698 | 55421 | NR | 1495 | 53915 | 51643 | 55508 | NR | 1895 | 150016 | 186277 | 148221 |
| 200 | NR | 44421 | 50054 | 56714 | NR | NR | 50884 | 50461 | 51048 | NR | NR | 165112 | 185182 | 136720 |
| 240 | NR | 49942 | 56378 | 55674 | NR | NR | NR | NR | NR | NR | NR | NR | NR | NR |
| 280 | NR | 50510 | 51713 | 54544 | NR | NR | NR | NR | NR | NR | NR | NR | NR | NR |

Values expressed in the table are relative light units (RLUs).
RA = rayon-tipped applicator,
CT = Clean-Trace surface ATP swab,
NR = not recorded.
BARDAC 205M beads, 205M-1p, if present, were added to the sample immediately before the first measurement was obtained.

### Example 5

### Detection of ATP from Suspensions of Live and Dead Microbial Cells Exposed to Hydrogel Beads Containing BARDAC 205M Antimicrobial

S. aureus and E. coli overnight cultures were prepared as described in Example 1. One milliliter of the overnight culture in tryptic soy broth (approximately 10⁹ CFU/ml) was boiled for 10 min to lyse the cells. Both the live and the dead cell suspensions were diluted to approximately 10⁷ and 10⁸ CFU/mL in Butterfield's buffer. 3M Clean-Trace surface ATP system swabs were replaced with sterile rayon-tipped applicators, as described in Example 1. Ten microliter amounts of live, dead, or mixtures of both live and dead bacterial suspensions were added directly to the rayon applicators or Clean-Trace surface ATP swabs. A BARDAC 205M hydrogel bead, 205M-1p, was added to the test units and each applicator or swab was inserted into a Clean-Trace surface ATP test unit to activate ATP detection according to the manufacturer's instructions. The test unit was inserted into a NG Luminometer, UNG2 instrument and RLU measurements were recorded at 15 sec intervals using the “Unplanned Testing” mode of the luminometer until the number of RLUs reached a plateau. The results are shown in Table 9. The RLU observed in samples containing dead cells reached maximum within about 30 sec and the addition of BARDAC beads did not result in a significant change in measurable RLUs. In samples containing both live and dead cells, the addition of BARDAC beads caused the RLU to increase relatively slowly over a period of several minutes, indicating that the beads caused the release of ATP from live cells. In contrast, tubes containing the Clean-Trace surface ATP swabs (which contain a cell extractant), showed an initial increase in RLU until a maximum was reached within about 30 seconds to 1 min.

TABLE 9
Detection of ATP from live and dead microbial cells exposed to
BARDAC 205M hydrogel beads.

| | S. aureus | | | | | E. coli | | | | |
|---|---|---|---|---|---|---|---|---|---|---|
| Time | Dead | Mixture | Live | Dead | Mixture | Dead | Mixture | Live | Dead | Mixture |
| (sec) | RA | RA | RA | CT | CT | RA | RA | RA | CT | CT |
| 15 | 3255 | 3330 | 1570 | 3657 | 17763 | 6817 | 8035 | 2070 | 6983 | 11136 |
| 30 | 3267 | 3460 | 2216 | 3691 | 20681 | 6787 | 8200 | 2112 | 7112 | 11278 |
| 45 | 3294 | 4636 | 2771 | 3708 | 22099 | 6756 | 8351 | 2255 | 7221 | 11323 |
| 60 | 3285 | 5143 | 3369 | 3738 | 22834 | 6749 | 8794 | 2322 | 7280 | 11352 |
| 90 | 3291 | 6369 | 4138 | 3792 | 22678 | 6780 | 10422 | 2373 | 7479 | 11319 |
| 120 | 3298 | 9254 | 4531 | 3853 | 22603 | 6761 | 12584 | 2412 | 7584 | 11310 |
| 150 | 3252 | 10760 | 5360 | 3898 | 22472 | 6756 | 13755 | 2420 | 7726 | 11344 |
| 180 | 3229 | 11535 | 9135 | 3922 | 22180 | 6827 | 14407 | 2423 | 7833 | 11219 |
| 210 | 3197 | 12577 | 9484 | 3967 | 22035 | 6862 | 14599 | 2475 | 7928 | 11153 |
| 240 | 3205 | 12801 | 9564 | 3988 | 21565 | 6851 | 14712 | 2472 | 8020 | 11098 |

Values expressed in the table are relative light units (RLUs).
RA = rayon-tipped applicator,
CT = Clean-Trace surface ATP swab.
BARDAC 205M beads (205-1p), if present, were added to the sample immediately before the first measurement was obtained.

### Example 6

### Detection of ATP from Suspensions of Microbial Cells Exposed to Hydrogel Beads Containing BARDAC 205M Antimicrobial in the Presence of Added Pure ATP

S. aureus and E. coli overnight cultures were prepared as described in Example 1. Immediately before use in these tests, the bacterial suspensions were diluted in Butterfield's buffer to concentrations of approximately 10⁸ CFU per milliliter. Luciferase/luciferin reagent (300 μl) from Clean-Trace surface ATP system was removed and added to 1.5 ml microfuge tubes. 100 nM solution of ATP (Sigma-Aldrich) was prepared in sterile water. Ten-microliter of ATP solution was added to individual microfuge tubes containing the reagents. Ten-microliter of the bacterial suspensions was added to some tubes containing reagents and ATP. BARDAC 205M hydrogel beads were prepared as described in Preparative Example 5 and one bead, 205M-1p, was added to some tubes. Relative Light Units (RLUs) were recorded at 10 sec interval in a bench top luminometer (FB-12 single tube luminometer with software), as described in Example 2. The results of the experiments are shown in Table 10. The results indicate that the addition of bacteria to pure ATP containing solution gave increased signal in the presence of BARDAC 205M beads. The extractants from beads were able to release ATP from cells leading to increased ATP levels which contribute to increased signal over the pure ATP background. Tubes containing no beads and bacteria did not show a significant increase in RLU's over that of pure ATP alone.

TABLE 10
Detection of ATP from microbial cells exposed to BARDAC
205M hydrogel beads in the presence of added pure ATP.

| | ATP 1 picomole | | ATP 1 picomole | |
|---|---|---|---|---|
| | No Bead | | 1 Bead | |
| Time | ATP | ATP + 10⁶ CFU | ATP | ATP + 10⁶ CFU |
| (sec) | alone | S. aureus | alone | S. aureus |
| 10 | 26985 | 28131 | 26890 | 31657 |
| 20 | 27223 | 28572 | 26823 | 32850 |
| 30 | 27423 | 28610 | 26931 | 124994 |
| 40 | 27325 | 28425 | 26980 | 184209 |
| 50 | 27030 | 28025 | 26640 | 243044 |
| 60 | 26995 | 27986 | 26525 | 340044 |
| 70 | NR | NR | NR | 466805 |
| 80 | NR | NR | NR | 561999 |
| 90 | NR | NR | NR | 600158 |
| 100 | NR | NR | NR | 631060 |

Values expressed in the table are relative light units (RLUs).
NR = not recorded.
BARDAC 205M beads, 205M-1p, if present, were added to the sample immediately before the first measurement was obtained.

### Example 7

### Detection of Live Microbial ATP in Milk

S. aureus overnight cultures were prepared as described in Example 1. BARDAC 205M beads were prepared as described in Preparative Example 5. Fresh, unpasteurized milk was obtained from a farm in River Falls, Wis. The milk was diluted with Butterfield's buffer (100-fold and 1000-fold). One hundred microliters of the diluted milk was mixed with 100 μl of luciferase/luciferin reagent from the Clean-Trace surface ATP system in a 1.5 ml tube and initial (T₀) luminescence measurements were recorded in a bench top luminometer (FB-12 single tube luminometer with software) as described in Example 2. After several measurements, one BARDAC 205M bead, 205M-1p, was added to milk and subsequent luminescence measurements were recorded at 10-second intervals. To other samples, S. aureus (approximately 10⁵ cells in 10 μL Butterfield's buffer) was added and, after taking the initial luminescence measurements, one 205M-1p bead was added to the sample. Subsequent luminescence measurements were recorded at 10-second intervals. The results are shown in Table 11. The data indicate that BARDAC beads were able to lyse bacteria spiked into milk and release ATP from cells, resulting in higher luminescence readings. The samples without added bacteria did not show a similar increase in luminescence after the BARDAC beads were added.

TABLE 11
Detection of S. aureus in milk samples.

| Time | 1:100 | 1:100 | 1:1000 | 1:1000 |
|---|---|---|---|---|
| (sec) | (no bacteria) | (with bacteria) | (no bacteria) | (with bacteria) |
| 0 | 19247 | 20015 | 7770 | 8600 |
| 10 | 19338 | 21230 | 7760 | 8810 |
| 20 | 19950 | 21460 | 7590 | 8330 |
| 30 | 19530 | 21000 | 7580 | 8200 |
| 40 | 18850 | 21140 | 7590 | 8810 |
| 50 | 19570 | 25390 | 7570 | 10800 |
| 60 | 21420 | 32190 | 8430 | 16420 |
| 70 | 21230 | 38250 | 8700 | 24090 |
| 80 | 21520 | 41876 | 8630 | 25180 |
| 90 | 21190 | 42910 | 8380 | 26310 |
| 100 | 21530 | 43830 | 8320 | 26580 |
| 110 | 21340 | 43840 | 8290 | 26880 |

BARDAC 205M bead, 205M-1p was added to the tubes immediately after the T₄₀ measurement was obtained. All measurements are reported in relative light units (RLU's).

### Example 8

### Distinguishing Microbial ATP from Somatic ATP

CRFK feline kidney cells (CCL-94, ATCC) were grown Dulbecco's Modified Eagle's Medium (DMEM) with 8% serum under CO₂ atmosphere at 37° C. to achieve 70% confluence. The medium was removed from the bottles and the cell monolayers were washed and were trypsinized (0.25% trypsin) for about 5 min. The detached cells were diluted with fresh medium and centrifuged at 3K for 5 min. The cells were further washed twice and resuspended in phosphate-buffered saline (PBS). The cells were diluted with PBS to get the desired cell concentration. One hundred microliters of cells were mixed with 100 μl of luciferase/luciferin reagent from Clean-Trace surface ATP system in a 1.5 ml tube. In one experiment, the tube was placed into a bench-top luminometer (FB-12 single tube luminometer with software), as described in Example 2, and initial luminescence measurements were recorded. After several initial measurements, one BARDAC 205M bead, 205M-1p, was added to the cell suspension and the luminescence was monitored at 10 sec intervals. In another experiment, S. aureus (approximately 10⁵ or 10⁶ cells in 10 μL of Butterfield's buffer) was added to the tube before the luminescence measurements were started. The results are shown in Table 10. The data indicate that BARDAC beads were able to cause the release of ATP from both mammalian cells and bacterial cells, resulting in an increased luminescence after the beads were added. In another experiment, the luminescence was monitored in a sample containing CRFK cells and a BARDAC bead. After 3 minutes, S. aureus cells were added to the same sample and luminescence was monitored for additional two minutes. The results, shown in Table 12, indicate that the amount of luminescence increased upon addition of S. aureus cells.

TABLE 12
Detection of ATP from somatic and microbial cells exposed to BARDAC 205M hydrogel bead.
BARDAC 205M hydrogel bead, 205M-1p, was added to the tubes immediately after the
T₄₀ measurement was obtained.

| | Experiment | | | | | | |
|---|---|---|---|---|---|---|---|
| | | | | | 5 | 6 | 7 |
| Time | 1 | 2 | 3 | 4 | CRFK (10⁴) + | CRFK (10⁴) + | CRFK (10⁵) + |
| (sec) | CRFK (10⁴) | CRFK (10⁵) | S. aureus (10⁵) | S. aureus (10⁶) | S. aureus (10⁵) | S. aureus (10⁵) | S. aureus (10⁶) |
| 0 | 31180 | 597030 | 1080 | 6030 | 33000 | 37769 | 583640 |
| 20 | 31870 | 593150 | 990 | 5990 | 33710 | 35757 | 585310 |
| 40 | 30100 | 585960 | 1090 | 6026 | 32790 | 33610 | 586920 |
| 60 | 31390 | 675559 | 3810 | 14413 | 32243 | 33130 | 868317 |
| 80 | 49970 | 678860 | 8450 | 23190 | 55110 | 49860 | 900480 |
| 100 | 49100 | 683520 | 10410 | 33890 | 80139 | 49150 | 918520 |
| 120 | 46380 | 697660 | 15889 | 45110 | 88900 | 47210 | 913270 |
| 140 | 45792 | 706010 | 32510 | 61800 | 100000 | 46025 | 903490 |
| 160 | 45691 | 714020 | 32950 | 80450 | 98450 | 45435 | 900860 |
| 180 | NR | NR | NR | NR | NR | 91048 | NR |
| 200 | NR | NR | NR | NR | NR | 101580 | NR |
| 220 | NR | NR | NR | NR | NR | 103230 | NR |
| 240 | NR | NR | NR | NR | NR | 99530 | NR |
| 260 | NR | NR | NR | NR | NR | 97403 | NR |
| 280 | NR | NR | NR | NR | NR | 97293 | NR |
| 300 | NR | NR | NR | NR | NR | 95340 | NR |

Values expressed in the table are relative light units (RLUs).
In Experiment 6, the S. aureus cells were added immediately after T = 160 measurement.
NR = not recorded.

### Example 9

### Detection of ATP from Live Microbial Cells in Food Extracts

Various food extracts (Spinach, Banana, and ground turkey) were prepared by adding 10 g to 100 ml of PBS in a stomacher bag and stomaching the food samples in a stomacher. 100 μl of spinach and banana extract and 100 μl diluted turkey extract (10-fold and 100-fold) were mixed with 100 μl of luciferase/luciferin reagent from Clean-Trace surface ATP system in a 1.5 ml microfuge tube and background readings were taken in a bench top luminometer (20/20n single tube luminometer, Turner Biosystems, Sunnyvale, Calif.). The initial (and all subsequent luminescence measurements) were obtained from the luminometer using 20/20n SIS software that was provided with the luminometer. The light signal was integrated for 1 second and the results are expressed in RLU/sec. After several readings, one BARDAC 205M bead, 205M-1p was added to the food extract and ATP release was monitored at 10 sec interval. The background levels were very high with banana and turkey extract and the levels increased upon addition of BARDAC bead. After 2 minutes, S. aureus cells (10⁵) were added to the same samples containing food extract and BARDAC bead and ATP release was monitored for additional four minutes. The ATP level increased upon addition of S. aureus cells (Table 13).

TABLE 13
Detection of ATP in food extracts.

| Time | Spinach | Banana | Turkey Extract | Turkey Extract |
|---|---|---|---|---|
| (sec) | Extract | Extract | (1:100) | (1:10) |
| 0 | 1063 | 150260 | 132670 | 997953 |
| 30 | 1081 | 130724 | 158942 | 1168784 |
| 60 | 1079 | 117705 | 172726 | 1284126 |
| 90 | 1093 | 105374 | 176684 | 1320036 |
| 120 | 1288 | 114530 | 155486 | 1599607 |
| 150 | 1316 | 121609 | 156589 | 1656526 |
| 180 | 1325 | 128329 | 157589 | 1746661 |
| 210 | 1391 | 140298 | 159553 | 1798493 |
| 240 | 10925 | 173838 | 177211 | 1930924 |
| 270 | 14730 | 176112 | 200387 | 2010237 |
| 300 | 18046 | 178565 | 212250 | 2088844 |
| 330 | 19607 | 182871 | 222775 | 2135284 |
| 360 | 20349 | 186227 | 229216 | 2178602 |
| 390 | 20549 | 190752 | 233637 | 2216695 |
| 420 | 20603 | 193788 | 238308 | 2265087 |
| 450 | 20600 | 197347 | 241146 | 2297345 |

BARDAC 205M bead, 205M-1p, was added to the tubes immediately after the T₁₀₀ measurement was obtained.
S. aureus cells were added to the tubes immediately after the T₂₂₀ measurement was obtained. All measurements are reported in relative light units (RLU's).

### Example 10

### Detection of ATP from Microbial Cells in Water

Overnight cultures of S. aureus were prepared as described in Example 1. Cooling tower water samples were obtained from two local cooling towers. One hundred microliters of water from each cooling tower was mixed with 100 μL of luciferin/luciferase reagent from Clean-Trace surface ATP system in individual 1.5 ml microfuge tubes. Luminescence was measured in a bench top luminometer (20/20n single tube luminometer with software) as described in Example 9, at 10-second intervals. After several measurements, one BARDAC 205M bead, 205M-1p, was added to the water sample and additional luminescence measurements were recorded to determine whether ATP was released from indigenous cells in the water samples. To other samples of cooling water from the same water towers, approximately 10⁵ CFU of S. aureus (suspended in 10 microliters of Butterfield's buffer) were added into individual 1.5 ml tubes containing the luciferin/luciferase reagent. The luminescence was measured in a bench top luminometer (20/20n single tube luminometer). After taking background (T₀) readings, one BARDAC 205M bead, 205M-1p, was added to the sample and luminescence was recorded at 10 second intervals. The results are shown in Table 14. The data indicate that the BARDAC beads were able to lyse bacteria spiked into water and release ATP from cells, causing an increase in luminescence over time.

TABLE 14
Detection of S. aureus in cooling tower water.

| Time | Cooling | Cooling Tower 1 + | Cooling | Cooling Tower 2 + |
|---|---|---|---|---|
| (sec) | Tower 1 | S. aureus | Tower 2 | S. aureus |
| 0 | 2652 | 3351 | 430 | 1211 |
| 10 | 2724 | 3387 | 427 | 1204 |
| 20 | 2768 | 3486 | 442 | 1202 |
| 30 | 2767 | 3525 | 440 | 1221 |
| 40 | 2922 | 3901 | 434 | 1270 |
| 50 | 2940 | 4371 | 621 | 2164 |
| 60 | 2997 | 5400 | 648 | 3151 |
| 70 | 3044 | 6586 | 666 | 4794 |
| 80 | 3110 | 7391 | 694 | 7809 |
| 90 | 3175 | 8014 | 725 | 10195 |
| 100 | 3214 | 8589 | 740 | 11972 |
| 110 | 3321 | 9228 | 772 | 13247 |

BARDAC 205M bead, 205M-1p, was added to the tubes containing cooling tower water samples immediately after recording T₀ measurement.
One 205M-1p bead was added to each tube containing cooling tower water spiked with S. aureus immediately after recording 40-second luminescence measurement.
All measurements are reported in relative light units (RLU's).

### Example 11

### Detection of ATP from Suspensions of Live Microbial Cells Exposed to Aqueous Extractants and Hydrogel Beads Containing Extractants

BARDAC 205M and 208M beads were produced as described in Preparative Example 5. 1 g of BARDAC 205M beads, 205M-1p, were added to 100 ml of distilled water and the water-soluble antimicrobial components were allowed to diffuse out of the beads and into the bulk solvent for 45 min. The beads were removed and the antimicrobial solution (“bead extract”) was saved. The amount of quaternary ammonium chloride (QAC) released was estimated using LaMotte QAC Test Kit Model QT-DR (LaMotte Company, Chester town, Md.). The amount of QAC released at the end of 45 min was 240 ppm.

A lysis solution (0.07% w/v Chlorhexidine digluconate (CHG, Sigma Aldrich) and 0.16% w/v Triton-X 100, Sigma Aldrich) was prepared in distilled water. A S. aureus overnight culture was prepared as described in Example 1 and the cells were diluted in Butterfield's buffer. One hundred microliters of luciferin/luciferase reagent from Clean-Trace surface ATP system was added to 1.5 ml microfuge tubes containing approximately 10⁵ cells. The lysis solution (25 or 50 μl) or bead extract (25 or 50 μl) was added to one of the microfuge tubes and the resulting luminescence was monitored in a bench top luminometer (20/20n single tube luminometer with software) as described in Example 9. To another set of samples one BARDAC 205M or 208M bead was added and the luminescence was monitored similarly. The results are shown in Table 15. The data indicate that the luminescence generated by the release of ATP from the bacteria was very gradual in samples that received the BARDAC beads. In contrast, samples that received either the lysis solution or the bead extract showed a rapid increase in luminescence, corresponding to a rapid release of ATP from the bacteria.

TABLE 15
Detection of ATP from cells exposed to a cell extractant
contained in a hydrogel or in an aqueous solution. All
measurements are reported in relative light units (RLU's).

| | | | | | 205M- | 205M- |
|---|---|---|---|---|---|---|
| | CHG Lysis | CHG Lysis | | | 1p Bead | 1p Bead |
| Time | Soln. | Soln. | 205M- | 208M- | Extract | Extract |
| (sec) | (25 μL) | (50 μL) | 1p bead | 1p bead | (25 μL) | (50 μL) |
| 0 | 1650 | 2243 | 853 | 881 | 918 | 932 |
| 10 | 15445 | 18232 | 1579 | 1930 | 5502 | 15288 |
| 20 | 16067 | 18771 | 2206 | 3453 | 10133 | 22579 |
| 30 | 16222 | 19156 | 3119 | 4881 | 17951 | 25554 |
| 40 | 16449 | 19314 | 4034 | 6583 | 26698 | 25795 |
| 50 | 16578 | 19501 | 4821 | 8215 | 28928 | 25964 |
| 60 | 16810 | 19629 | 5550 | 9814 | 29397 | 25895 |
| 80 | 16940 | 19839 | 7538 | 12910 | 30943 | 26203 |
| 100 | 17162 | 19903 | 8738 | 14074 | 32032 | 26125 |
| 120 | 17251 | 20050 | 9690 | 15049 | 32854 | 26204 |
| 140 | 17413 | 20180 | 10363 | 16259 | 33441 | 26137 |
| 160 | 17375 | 20233 | 10919 | 16737 | 33647 | 26042 |
| 180 | 17330 | 20076 | 11190 | 17096 | 33663 | 26041 |

### Example 12

### Detection of ATP from Suspensions of Microbial Cells Exposed to Hydrogel Beads Containing Various Amounts of Extractants

Hydrogel beads with various amounts of BARDAC 205M or 208M were prepared as described in Preparative Example 5. S. aureus and E. coli overnight cultures were prepared as described in Example 1. One hundred microliters of luciferin/luciferase reagent from Clean-Trace surface ATP system was added to individual 1.5 ml microfuge tubes containing approximately 10⁵ CFU of one of the respective bacterial cultures. One bead or Clean-Trace surface ATP swab was added to each tube. Luminescence, resulting from the release of ATP from the cells, was recorded at 10 sec intervals in a bench top luminometer (20/20n single tube luminometer with software) as described in Example 9. The results are shown in Tables 16 and 17. The data indicate that ATP release was very gradual in the samples containing the beads. In contrast, samples containing the swabs (which contain a cell extractant solution) showed a very rapid release of ATP from the cells.

TABLE 16
Detection of S. aureus using hydrogel beads containing
BARDAC 205M or BARDAC 208M antimicrobial mixtures.

| Time | 205M-1p | 205M-2p | 208M-1p | 208M-2p | |
|---|---|---|---|---|---|
| (sec) | bead | bead | bead | bead | CT Swab |
| 0 | 377 | 537 | 484 | 427 | 489 |
| 10 | 1126 | 1816 | 2055 | 951 | 17746 |
| 20 | 1215 | 2624 | 6585 | 1116 | 20330 |
| 30 | 1299 | 4738 | 15094 | 1474 | 21886 |
| 40 | 1492 | 8709 | 21706 | 2035 | 23172 |
| 50 | 1870 | 13511 | 26156 | 2845 | 23444 |
| 60 | 2339 | 18283 | 29355 | 4013 | 23483 |
| 80 | 3622 | 29767 | 32316 | 10224 | 23580 |
| 100 | 4933 | 33298 | 32894 | 14878 | 23544 |
| 120 | 6434 | 34126 | 31614 | 19264 | 23389 |
| 140 | 8439 | 33810 | 30164 | 23320 | 23407 |
| 160 | 10420 | 31938 | 28664 | 27478 | 23282 |
| 180 | 13013 | 30078 | 27085 | 29058 | 23197 |

Hydrogel beads containing BARDAC mixtures were added to the tubes immediately after the T₀ measurement was recorded.
All measurements are reported in relative light units (RLU's).

TABLE 17
Detection of E. coli using hydrogel beads containing
BARDAC 205M or BARDAC 208M antimicrobial mixtures.

| Time | 205M-1p | 205M-2p | 208M-1p | 208M-2p | |
|---|---|---|---|---|---|
| (sec) | bead | bead | bead | bead | CT Swab |
| 0 | 484 | 508 | 635 | 685 | 886 |
| 10 | 699 | 1427 | 2507 | 1464 | 40823 |
| 20 | 717 | 1656 | 3038 | 1615 | 42986 |
| 30 | 728 | 1996 | 3888 | 1975 | 43125 |
| 40 | 770 | 2982 | 5681 | 2525 | 43274 |
| 50 | 936 | 5250 | 9546 | 3614 | 43275 |
| 60 | 1020 | 8762 | 15512 | 5606 | 43084 |
| 80 | 1321 | 17693 | 28302 | 14018 | 42869 |
| 100 | 1678 | 24646 | 39101 | 20923 | 42779 |
| 120 | 2185 | 27352 | 40693 | 27997 | 42677 |
| 140 | 2757 | 28165 | 40612 | 34621 | 42512 |
| 160 | 3436 | 28131 | 39926 | 36797 | 42360 |
| 180 | 4193 | 28010 | 38988 | 37846 | 42215 |

Hydrogel beads containing BARDAC mixtures were added to the tubes immediately after the T₀ measurement was recorded.
All measurements are reported in relative light units (RLU's).

### Example 13

### Release of ATP from S. aureus Exposed to Various Antimicrobial-Loaded Hydrogel Beads

Hydrogel beads with various amounts of BARDAC 205M or 208M were prepared as described in Preparative Example 1. A S. aureus overnight culture was prepared as described in Example 1. Microfuge tubes (1.5 mL) were prepared by adding 100 microliters of luciferin/luciferase reagent from Clean-Trace surface ATP system. 100 microliters of the diluted suspension were added directly to individual microfuge tubes (i.e., approximately 10⁵ CFU per tube). Immediately after adding the bacterial suspension, the tube was placed into a bench-top luminometer (20/20n single tube luminometer with software), as described in Example 9, and an initial (T₀) measurement of RLUs was recorded. A hydrogel bead containing extractants was added to individual tubes and RLU measurements were recorded at 10 sec intervals until the number of RLUs reached a plateau or began to decrease (Table 18). The data indicate that all four of the bead formulations caused the release of ATP from the microbial cells.

TABLE 18
Release of ATP from S. aureus after exposure of the bacteria to
antimicrobial-loaded hydrogels.

| Time | 205M-2s | 208M-2s | 205M-1s | |
|---|---|---|---|---|
| (sec) | bead | bead | bead | 208M-1s bead |
| 0 | 1099 | 990 | 2053 | 1198 |
| 10 | 2025 | 2073 | 3573 | 1228 |
| 20 | 9442 | 3074 | 5921 | 1313 |
| 30 | 16070 | 4063 | 8757 | 1517 |
| 40 | 22844 | 5136 | 12056 | 1761 |
| 50 | 27610 | 6186 | 14748 | 2090 |
| 60 | 29653 | 7222 | 16417 | 2481 |
| 70 | 29906 | 8484 | 17095 | 2802 |
| 80 | 29453 | 9420 | 16979 | 3224 |
| 90 | 28449 | 10259 | 16524 | 3618 |
| 100 | 27396 | 11176 | 15723 | 3987 |
| 110 | 26152 | 11765 | 15062 | 4355 |
| 120 | 25039 | 12601 | 14586 | 4699 |

All data are reported in relative light units (RLU's).
BARDAC hydrogel beads were added to the sample immediately after the T₀ measurement was obtained.

### Example 14

### Release of ATP from Various Microbial Cells Exposed to Antimicrobial-Loaded Hydrogel Beads

Hydrogel beads with various amounts of BARDAC 205M or 208M were prepared as described in Preparative Example 5. Cultures of S. aureus, P. aeruginosa and S. epidermidis were prepared as described in Example 1. Microfuge tubes (1.5 mL) were prepared by adding 100 microliters of luciferin/luciferase reagent from Clean-Trace surface ATP system. 100 microliters of the diluted suspension were added directly to individual microfuge tubes (i.e., approximately 10⁵ CFU per tube). Immediately after adding the bacterial suspension, the tube was placed into a bench-top luminometer (20/20n single tube luminometer with software), as described in Example 9, and an initial (T₀) measurement of RLUs was recorded. A hydrogel bead containing extractants was added to individual tubes and RLU measurements were recorded at 10 sec intervals until the number of RLUs reached a plateau or began to decrease (Table 19). The data indicate that all of the bead formulations caused the release of ATP from the microbial cells.

TABLE 19
Release of ATP from microbial cells after exposure of the bacteria to antimicrobial-loaded hydrogels.
All data are reported in relative light units (RLU's). BARDAC hydrogel beads were added
to the sample immediately after the T₀ measurement was obtained.

| | S. aureus | | | | P. aeruginosa | | | | S. epidermidis | |
|---|---|---|---|---|---|---|---|---|---|---|
| Time | 205M- | 208M- | 205M- | 208M- | 205M- | 208M- | 205M- | 208M- | 205M- | 208M- |
| (sec) | 5p bead | 3p bead | 3p bad | 2p bead | 5p bead | 3p bead | 3p bad | 2p bead | 5p bead | 3p bead |
| 0 | 1099 | 990 | 2053 | 1198 | 5799 | 2922 | 2523 | 1699 | 2273 | 2117 |
| 10 | 2025 | 2073 | 3573 | 1228 | 7426 | 3112 | 15190 | 11977 | 3217 | 2383 |
| 20 | 9442 | 3074 | 5921 | 1313 | 9107 | 3197 | 13717 | 11271 | 6444 | 4132 |
| 30 | 16070 | 4063 | 8757 | 1517 | 11267 | 3369 | 12320 | 10279 | 11060 | 6840 |
| 40 | 22844 | 5136 | 12056 | 1761 | 14585 | 3735 | 10884 | 8971 | 15496 | 10587 |
| 50 | 27610 | 6186 | 14748 | 2090 | 17849 | 4337 | 9583 | 7989 | 19211 | 13437 |
| 60 | 29653 | 7222 | 16417 | 2481 | 20063 | 4934 | 8343 | 6987 | 21743 | 14011 |
| 70 | 29906 | 8484 | 17095 | 2802 | 21050 | 5511 | 7325 | 6255 | 23296 | 13493 |
| 80 | 29453 | 9420 | 16979 | 3224 | 20662 | 5938 | 6423 | 5509 | 23954 | 12429 |
| 90 | 28449 | 10259 | 16524 | 3618 | 20369 | 6255 | 5262 | 4913 | 24056 | 11839 |
| 100 | 27396 | 11176 | 15723 | 3987 | 19632 | 6340 | 4677 | 4389 | 23700 | 11231 |

| | S. epidermidis | | S. enterica subsp. enterica | | | |
|---|---|---|---|---|---|---|
| Time | 205M- | 208M- | 205M- | 208M- | 205M- | 208M- |
| (sec) | 3p bead | 2p bead | 5p bead | 3p bead | 3p bead | 2p bead |
| 0 | 4378 | 445 | 1091 | 4265 | 5164 | 3142 |
| 10 | 6376 | 424 | 1080 | 8676 | 8570 | 5409 |
| 20 | 8468 | 444 | 1230 | 9309 | 9208 | 8460 |
| 30 | 10863 | 471 | 1701 | 9235 | 9244 | 9642 |
| 40 | 12958 | 480 | 2275 | 8658 | 8804 | 9708 |
| 50 | 14564 | 499 | 2879 | 8076 | 8248 | 9369 |
| 60 | 15991 | 532 | 3496 | 6929 | 7644 | 8971 |
| 70 | 16231 | 665 | 4057 | 6370 | 7109 | 8388 |
| 80 | 16383 | 917 | 4687 | 5922 | 6551 | 7471 |
| 90 | 16044 | 996 | 5966 | 5428 | 6121 | 6652 |
| 100 | 15708 | 1053 | 6247 | 5026 | 5759 | 6007 |

### Example 15

### Release of ATP from Various Microbial Cells Exposed to BARDAC 205M Hydrogel Beads

Hydrogel bead with 50% solution of BARDAC 205M was prepared as described in Preparative Example 5. Cultures of a number of different microorganisms were prepared as described in Example 1. Microfuge tubes (1.5 mL) were prepared by adding 100 microliters of luciferin/luciferase reagent from Clean-Trace surface ATP system. 100 microliters of the diluted suspension were added directly to individual microfuge tubes (i.e., approximately 10⁵ or 10⁶ or 10⁷ CFU per tube). Immediately after adding the bacterial suspension, the tube was placed into a bench-top luminometer (20/20n single tube luminometer with software), as described in Example 9, and an initial (T₀) measurement of RLUs was recorded. A hydrogel bead made from 50% BARDAC 205M solution, 205M-2p, was added to individual tubes and RLU measurements were recorded at 10 sec intervals until the number of RLUs reached a plateau or began to decrease (Table 20). The data indicate that the hydrogel bead containing BARDAC 205M caused the release of ATP from a variety of microbial cells.

TABLE 20
Release of ATP from microbial cells after exposure of the bacteria to BARDAC 205M, 205M-2p, hydrogel beads. All data are
reported in relative light units (RLU's). 205M-2p beads were added to the sample immediately after the T₀ measurement was obtained.

| | 10⁵ CFU | | | | | | | 10⁶ CFU | | | | |
|---|---|---|---|---|---|---|---|---|---|---|---|---|
| | | | K. | E. | | | E. | C. | S. | | 10⁷ CFU | |
| Time | C. albicans | C. albicans | kristinae | faecium | E. faecium | E. faecalis | faecalis | xerosis | pneumoniae | S. aureus | S. aureus | M. luteus |
| (sec) | MYA-2876 | 10231 | BAA-752 | 6569 | 700221 | 49332 | 700802 | 373 | 6301 | 6538 | 6538 | 540 |
| 0 | 5161 | 4125 | 3762 | 145661 | 43780 | 1649 | 36858 | 3482 | 3306 | 1394 | 12079 | 44909 |
| 10 | 16987 | 11776 | 4000 | 153959 | 155023 | 14727 | 44451 | 16180 | 8462 | 2308 | 25805 | 52335 |
| 20 | 21356 | 21991 | 50137 | 170058 | 285666 | 29188 | 63119 | 24878 | 11582 | 3091 | 35028 | 57498 |
| 30 | 28823 | 44325 | 82543 | 209621 | 349995 | 50927 | 123300 | 31381 | 15338 | 5774 | 51282 | 66746 |
| 40 | 43630 | 67484 | 128571 | 260697 | 386112 | 77724 | 212171 | 37995 | 20488 | 10499 | 67125 | 93143 |
| 50 | 65132 | 86962 | 194474 | 301252 | 408647 | 107231 | 300942 | 47178 | 26440 | 17387 | 93337 | 142442 |
| 60 | 86624 | 105048 | 267643 | 333434 | 425346 | 134214 | 365570 | 60455 | 32002 | 27294 | 222624 | 199757 |
| 70 | 107992 | 124236 | 341048 | 358913 | 449577 | 157280 | 408127 | 75323 | 37780 | 38335 | 319768 | 257819 |
| 80 | 131101 | 144606 | 411850 | 379094 | 459167 | 176794 | 456630 | 90074 | 44291 | 49969 | 401580 | 317016 |
| 90 | 157399 | 166654 | 477954 | 395504 | 467340 | 192530 | 470824 | 104404 | 51766 | 61759 | 491648 | 378876 |
| 100 | 190305 | 187641 | 537507 | 408552 | 474223 | 216918 | 483047 | 120709 | 60659 | 73799 | 575341 | 444460 |
| 120 | 228718 | 209062 | 592031 | 419596 | 480639 | 226425 | 492008 | 138375 | 70014 | 85640 | 675291 | 506650 |
| 130 | 317509 | 232116 | 639725 | 428420 | 485000 | 235454 | 500264 | 157147 | 79893 | 96589 | 877112 | 559625 |
| 140 | 363503 | 255940 | 684210 | 436263 | 490791 | 243147 | 506623 | 175508 | 90109 | 106625 | 950544 | 602616 |
| 150 | 410236 | 277256 | 723642 | 441677 | 492648 | 250465 | 512667 | 191248 | 100082 | 115289 | 1020667 | 637557 |
| 160 | 460327 | 296434 | 758988 | 445742 | 493961 | 256537 | 517304 | 210910 | 110172 | 121803 | 1085813 | 664924 |
| 170 | 510335 | 313496 | 791605 | 449138 | 494542 | 264996 | 521471 | 215701 | 119760 | 126891 | 1131039 | 688074 |
| 180 | 559811 | 328745 | 819710 | 451383 | 494909 | 267500 | 524029 | 218404 | 129408 | 130700 | 1164549 | 707790 |

### Example 16

### Detection of ATP from Suspensions of Microbial Cells Exposed to BARDAC 205M Containing Hydrogel Beads with Continuous Mixing and no Mixing

Hydrogel bead with 50% solution of BARDAC 205M, 205M-2p, was prepared as described in Preparative Example 5. S. aureus and E. coli overnight cultures were prepared as described in Example 1. One hundred microliters of luciferin/luciferase reagent from Clean-Trace surface ATP system was added to individual 1.5 ml microfuge tubes containing approximately 10⁵ or 10⁶ CFU of one of the respective bacterial cultures. Immediately after adding the bacterial suspension, the tube was placed into a bench-top luminometer (20/20n single tube luminometer with software), as described in Example 9, and an initial (T₀) measurement of RLUs was recorded. One 205M-1p bead was added to each tube. One set of tubes were vortexed for 5 sec between each reading and luminescence resulting from the release of ATP from the cells, was recorded at 10 sec intervals. The other set of tubes were not vortexed, but allowed to sit for 5 sec between each readings. The results are shown in Table 21. The data indicate that ATP release was very rapidly in tubes that were mixed and very gradually in the samples that were not mixed.

TABLE 21
Detection of S. aureus and E. coli using hydrogel beads containing
BARDAC 205M antimicrobial mixtures. BARDAC 205M bead, 205M-2p was
added to the tubes immediately after the T₀ measurement was recorded. For the
vortexing experiment, the tubes were vortexed for 5 sec before each
measurement. For no vortexing experiment, the tubes were allowed to sit for 5
sec before recording each measurement. All measurements are reported in
relative light units (RLU's).

| | S. aureus | | | | E. coli | | | |
|---|---|---|---|---|---|---|---|---|
| | | | 10⁶ CFU | | | | 10⁶ CFU | |
| Time | 10⁵ CFU | | | No | 10⁵ CFU | | | No |
| (sec) | Vortex | No vortex | Vortex | Vortex | Vortex | No vortex | Vortex | vortex |
| 0 | 305 | 498 | 2005 | 2091 | 463 | 580 | 1906 | 1823 |
| 10 | 1006 | 826 | 9195 | 3734 | 1445 | 1488 | 13743 | 6104 |
| 20 | 2864 | 1010 | 42528 | 12846 | 2197 | 1615 | 18709 | 6867 |
| 30 | 14239 | 1359 | 223585 | 23113 | 4232 | 1775 | 29616 | 7363 |
| 40 | 31719 | 2832 | 387510 | 54554 | 12623 | 2082 | 112254 | 10903 |
| 50 | 53347 | 6246 | 570830 | 107449 | 17823 | 2493 | 193775 | 12743 |
| 60 | 69178 | 11550 | 643850 | 182751 | 18410 | 5780 | 195720 | 14176 |
| 70 | 74075 | 19119 | 654632 | 258945 | 17600 | 7299 | 192598 | 16919 |
| 80 | 74932 | 27536 | 644469 | 327138 | 16939 | 10271 | 188490 | 22830 |
| 90 | 73404 | 35364 | 637619 | 407092 | 16507 | 12478 | 182579 | 35515 |
| 100 | 71450 | 42173 | 618062 | 475468 | 15725 | 15344 | 176829 | 55633 |
| 110 | 67412 | 49205 | 588024 | 563239 | 15062 | 18152 | 172002 | 77675 |
| 120 | 62889 | 55253 | 604301 | 613548 | 13983 | 20871 | 175739 | 103648 |
| 130 | 58353 | 61832 | 583681 | 678871 | 13893 | 23163 | 169994 | 147703 |
| 140 | 55479 | 67893 | 574342 | 754416 | 13204 | 24771 | 170803 | 174745 |
| 150 | 52797 | 72663 | 580001 | 829087 | 12325 | 26561 | 167078 | 193287 |
| 160 | 50302 | 76902 | 557410 | 878127 | 12565 | 27572 | 156931 | 223821 |

### Example 17

### Detection of ATP from Suspensions of Microbial Cells Exposed to Crushed and Uncrushed BARDAC 205M Containing Hydrogel Beads

S. aureus and E. coli overnight cultures were prepared as described in Example 1. One hundred microliters of luciferin/luciferase reagent from Clean-Trace surface ATP system was added to individual 1.5 ml microfuge tubes containing approximately 10⁵ or 10⁶ CFU of one of the respective bacterial cultures. Immediately after adding the bacterial suspension, the tube was placed into a bench-top luminometer (20/20n single tube luminometer with software), as described in Example 9, and an initial (T₀) measurement of RLUs was recorded. One BARDAC 205M bead, 205M-2p was added to each tube and in one set of tubes the beads were crushed using the blunt end of a sterile cotton swab. Luminescence, resulting from the release of ATP from the cells, was recorded at 10 sec intervals. The results are shown in Table 22. The data indicate that the crushed beads rapidly released ATP from cells unlike uncrushed beads which showed a gradual increase in ATP levels.

TABLE 22
Detection of S. aureus and E. coli using hydrogel beads containing BARDAC 205M
antimicrobial mixtures. BARDAC bead, 205M-2p was added to the tubes immediately
after the T₀ measurement was recorded. For crushed bead experiment the bead was
crushed immediately after T₀ measurement with the blunt end of a sterile cotton
swab. All measurements are reported in relative light units (RLU's).

| | S. aureus | | | | E. coli | | | |
|---|---|---|---|---|---|---|---|---|
| | 10⁵ CFU | | 10⁶ CFU | | 10⁵ CFU | | 10⁶ CFU | |
| Time | Uncrushed | Crushed | Uncrushed | Crushed | Uncrushed | Crushed | Uncrushed | Crushed |
| (sec) | Bead | Bead | Bead | Bead | Bead | Bead | Bead | Bead |
| 0 | 755 | 569 | 1434 | 1685 | 661 | 921 | 1547 | 1548 |
| 10 | 1717 | 23912 | 7813 | 90362 | 3065 | 20831 | 17151 | 95027 |
| 20 | 1826 | 44857 | 9584 | 160602 | 5298 | 23054 | 36596 | 165658 |
| 30 | 2106 | 50007 | 12476 | 211406 | 6841 | 23123 | 57201 | 205345 |
| 40 | 2582 | 49632 | 17628 | 255960 | 7973 | 22404 | 75033 | 245350 |
| 50 | 3504 | 47961 | 24936 | 278480 | 9347 | 21510 | 91107 | 282125 |
| 60 | 5103 | 45779 | 38234 | 281923 | 10930 | 20218 | 132156 | 235422 |
| 70 | 7299 | 43708 | 54223 | 276518 | 12637 | 18474 | 155682 | 202187 |
| 80 | 10201 | 41601 | 68084 | 266337 | 14294 | 16633 | 177966 | 175465 |
| 90 | 13371 | 39292 | 86533 | 253028 | 16003 | 14940 | 200829 | 152480 |
| 100 | 16581 | 37091 | 113368 | 237359 | 17748 | NR | 222834 | NR |
| 110 | 19865 | NR | 142674 | NR | 19399 | NR | 244224 | NR |
| 120 | 25670 | NR | 171218 | NR | 22426 | NR | 288422 | NR |
| 130 | 28060 | NR | 197768 | NR | 23497 | NR | 308373 | NR |
| 140 | 30086 | NR | 223874 | NR | 24529 | NR | 322147 | NR |
| 150 | 31676 | NR | 251004 | NR | 25028 | NR | 331004 | NR |
| 160 | 33231 | NR | 274659 | NR | 25531 | NR | 335211 | NR |
| 170 | 34626 | NR | 299417 | NR | 25843 | NR | 336701 | NR |
| 180 | 35942 | NR | 323084 | NR | 26050 | NR | 340089 | NR |
| 190 | 36809 | NR | 348944 | NR | 26157 | NR | 339987 | NR |
| 200 | 37804 | NR | 370478 | NR | 26469 | NR | 340442 | NR |
| 210 | 38582 | NR | 388143 | NR | 26451 | NR | 340842 | NR |
| 220 | 39364 | NR | 404821 | NR | 26615 | NR | 341181 | NR |
| 230 | 39905 | NR | 416442 | NR | 26824 | NR | 340627 | NR |
| 240 | 40344 | NR | 427181 | NR | 26766 | NR | 338149 | NR |

NR = Not recorded.

### Example 18

### Detection of ATP from Suspensions of Microbial Cells exposed to Hydrogel Beads Containing Various Extractants

Hydrogel beads with various amounts of chlorhexidine digluconate (CHG) or Cetyl trimethylammonium bromide (CTAB) and Triclosan were prepared as described in Preparative Example 5. S. aureus and E. coli overnight cultures were prepared as described in Example 1. One hundred microliters of luciferin/luciferase reagent from Clean-Trace surface ATP system was added to individual 1.5 ml microfuge tubes containing approximately 10⁶ CFU of one of the respective bacterial cultures. Immediately after adding the bacterial suspension, the tube was placed into a bench-top luminometer (20/20n single tube luminometer with software), as described in Example 9, and an initial (T₀) measurement of RLUs was recorded. One bead containing the extractant was added to each tube. Luminescence, resulting from the release of ATP from the cells, was recorded at 10 sec intervals. The results are shown in Tables 23. The data indicate that CHG, CTAB and Triclosan beads were able to release ATP from cells.

TABLE 23
Detection of S. aureus and E. coli using hydrogel beads containing various extractants.
Beads containing extractants were added to the tubes immediately after the T₀ measurement was recorded.
All measurements are reported in relative light units (RLU's).

| | S. aureus | | | | | E. coli | | | | |
|---|---|---|---|---|---|---|---|---|---|---|
| Time (sec) | CHG-1p | CHG-2p | CTAB-1p | CTAB-2p | Triclosan-1p | CHG-1p | CHG-2p | CTAB-1p | CTAB-2p | Triclosan-1p |
| 0 | 532 | 1875 | 985 | 937 | 1425 | 1049 | 1211 | 844 | 1197 | 650 |
| 10 | 2950 | 10184 | 1244 | 1160 | 2906 | 2193 | 8234 | 911 | 1561 | 1594 |
| 20 | 5045 | 14078 | 1322 | 1259 | 3067 | 3038 | 14288 | 973 | 1584 | 1906 |
| 30 | 8615 | 17165 | 1492 | 1368 | 3201 | 4335 | 21251 | 989 | 1624 | 2265 |
| 40 | 10248 | 19891 | 1810 | 1476 | 3453 | 5894 | 30499 | 1036 | 1703 | 2756 |
| 50 | 11790 | 22362 | 1959 | 1609 | 3768 | 7690 | 40819 | 1117 | 1748 | 3424 |
| 60 | 13420 | 24836 | 2102 | 1734 | 4495 | 9548 | 65544 | 1177 | 1814 | 4145 |
| 70 | 15024 | 27116 | 2256 | 1866 | 4874 | 11558 | 79557 | 1242 | 1930 | 4868 |
| 80 | 16697 | 29353 | 2401 | 1986 | 5273 | 13641 | 93910 | 1327 | 1995 | 5656 |
| 90 | 18293 | 31330 | 2587 | 2131 | 5691 | 15757 | 108312 | 1391 | 2095 | 6415 |
| 100 | 19924 | 33402 | 2741 | 2279 | 6138 | 17862 | 122120 | 1608 | 2169 | 7150 |
| 110 | 21562 | 35642 | 2957 | 2400 | 6620 | 20219 | 135239 | 1685 | 2308 | 7944 |
| 120 | 23225 | 37478 | 3098 | 2596 | 7035 | 22528 | 159694 | 1792 | 2421 | 8738 |
| 130 | 24904 | 39402 | 3298 | 2768 | 7385 | 25077 | 170749 | 1875 | 2515 | 9425 |
| 140 | 26453 | 41107 | 3477 | 2971 | 7858 | 27749 | 181638 | 1984 | 2626 | 10142 |
| 150 | 28095 | 42896 | 3720 | 3158 | 8281 | 30435 | 191090 | 2087 | 2761 | 10924 |
| 160 | 29673 | 44621 | 3925 | 3322 | 8688 | 33344 | 200990 | 2314 | 2873 | 11619 |
| 180 | 31203 | 46286 | 4353 | 3545 | 9111 | 36249 | 210178 | 2423 | 3026 | 12374 |
| 190 | 32666 | 47911 | 4598 | 3779 | 9585 | 39263 | 218677 | 2538 | 3158 | 13198 |
| 200 | 34244 | 49513 | 4854 | 4006 | 10310 | 42268 | 226765 | 2625 | 3280 | 13868 |
| 210 | 35728 | 51022 | 5084 | 4239 | 10779 | 45511 | 234148 | 2742 | 3436 | 14597 |
| 220 | 37071 | 52500 | 5318 | 4480 | 11286 | 48709 | 241269 | 2868 | 3603 | 15161 |
| 230 | 38586 | 53808 | 5571 | 4691 | 11761 | 51994 | 247902 | 3042 | 3755 | 15938 |
| 240 | 40048 | 55096 | 6135 | 4933 | 12147 | 55230 | 254243 | 3138 | 3933 | 16572 |

### Example 19

### Detection of ATP from Suspensions of Microbial Cells Exposed to Hydrogel Beads Containing Cationic Monomers

Hydrogel beads with cationic monomers were prepared as described in Preparative Example 2. S. aureus and E. coli overnight cultures were prepared as described in Example 1. One hundred microliters of luciferin/luciferase reagent from Clean-Trace surface ATP system was added to individual 1.5 ml microfuge tubes containing approximately 10⁶ CFU of one of the respective bacterial cultures. Immediately after adding the bacterial suspension, the tube was placed into a bench-top luminometer (20/20n single tube luminometer with software), as described in Example 9, and an initial (T₀) measurement of RLUs was recorded. One bead containing the extractant was added to each tube. Luminescence, resulting from the release of ATP from the cells, was recorded at 10 sec intervals. The results are shown in Table 24. The data indicate that beads containing cationic monomers were able to release ATP from cells.

TABLE 24
Detection of S. aureus and E. coli using hydrogel beads containing cationic monomers.
Beads containing extractants were added to the tubes immediately after the T₀ measurement was recorded.
All measurements are reported in relative light units (RLU's).

| Time | S. aureus | | | | | E. coli | | | | |
|---|---|---|---|---|---|---|---|---|---|---|
| (sec) | C₈-1s | C₁₀-1s | C₁₂-1s | ATAC-1s | Ageflex-1s | C₈-1s | C₁₀-1s | C₁₂-1s | ATAC-1s | Ageflex-1s |
| 0 | 1632 | 1388 | 945 | 1379 | 1319 | 790 | 945 | 1056 | 760 | 982 |
| 10 | 2388 | 4136 | 1886 | 2540 | 3427 | 1596 | 1845 | 1974 | 1159 | 1527 |
| 20 | 2995 | 6939 | 2556 | 2661 | 3771 | 1831 | 1889 | 2518 | 1199 | 1539 |
| 30 | 3563 | 8198 | 2717 | 2784 | 3917 | 2009 | 1928 | 2898 | 1219 | 1555 |
| 40 | 4128 | 9390 | 2801 | 2902 | 4064 | 2091 | 3206 | 3059 | 1298 | 1597 |
| 50 | 4728 | 10603 | 2825 | 3073 | 4237 | 2206 | 3602 | 3187 | 1314 | 1618 |
| 60 | 5346 | 11779 | 2875 | 3192 | 4424 | 2256 | 3919 | 3241 | 1366 | 1648 |
| 70 | 5814 | 12945 | 2912 | 3340 | 4579 | 2284 | 4257 | 3319 | 1356 | 1686 |
| 80 | 6318 | 14071 | 2906 | 3535 | 4900 | 2321 | 4531 | 3337 | 1392 | 1705 |
| 90 | 6873 | 15223 | 2951 | 3696 | 5039 | 2364 | 5074 | 3405 | 1401 | 1741 |
| 100 | 7300 | 16414 | 2945 | 3836 | 5196 | 2396 | 5329 | 3369 | 1498 | 1737 |
| 110 | 7741 | 17496 | 2984 | 4009 | 5330 | 2402 | 5598 | 3334 | 1530 | 1764 |
| 120 | 8153 | 18497 | 3001 | 4135 | 5395 | 2450 | 5872 | 3327 | 1541 | 1790 |
| 130 | 8629 | 19601 | 3018 | 4261 | 5593 | 2468 | 6104 | 3279 | 1616 | 1824 |
| 140 | 8948 | 20727 | 3060 | 4439 | 5877 | 2498 | 6380 | 3242 | 1639 | 1840 |
| 150 | 9415 | 22776 | 3090 | 4592 | 5949 | 2545 | 6866 | 3229 | 1702 | 1864 |
| 160 | 9702 | 24009 | 3105 | 4692 | 6058 | 2545 | 7134 | 3197 | 1736 | 1881 |
| 170 | 10085 | 25035 | 3048 | 4880 | 6176 | 2548 | 7379 | 3102 | 1757 | 1893 |
| 180 | 10429 | 26159 | NR | 4995 | 6316 | 2568 | 7886 | NR | 1844 | 1956 |
| 190 | 10738 | 28310 | NR | 5109 | 6347 | 2514 | 8091 | NR | 1846 | 1956 |
| 200 | 11060 | 29416 | NR | 5257 | 6584 | 2499 | 8293 | NR | 1849 | 1967 |
| 210 | 11351 | 30469 | NR | 5394 | 6681 | 2457 | 8579 | NR | 1949 | 1999 |
| 220 | 11589 | 31536 | NR | 5563 | 6764 | 2475 | 8748 | NR | 1930 | 2030 |
| 230 | 12012 | 32585 | NR | 5617 | 6818 | 2474 | 8801 | NR | 2043 | 2061 |
| 240 | 12265 | 33561 | NR | 5739 | 6859 | 2461 | 9048 | NR | 2022 | 2080 |

NR = not recorded

### Example 20

### Detection of ATP from Suspensions of Microbial Cells Exposed to Hydrogel Fibers Containing Microbial Extractant

Hydrogel fibers were prepared as described in Preparative Example 6. S. aureus and E. coli overnight cultures were prepared as described in Example 1. One hundred microliters of luciferin/luciferase reagent from Clean-Trace surface ATP system was added to individual 1.5 ml microfuge tubes containing approximately 10⁵ or 10⁶ CFU of one of the respective bacterial cultures. Immediately after adding the bacterial suspension, the tube was placed into a bench-top luminometer (20/20n single tube luminometer with software), as described in Example 9, and an initial (Tₒ) measurement of RLUs was recorded. About 5 mg of hydrogel fiber containing the extractant was added to each tube. Luminescence, resulting from the release of ATP from the cells, was recorded at 10 sec intervals. The results are shown in Table 25. The data indicate that fibers containing microbial extractant were able to release ATP from cells.

TABLE 25
Detection of S. aureus and E. coli using hydrogel
fibers containing BARDAC 205M.

| | | |
|---|---|---|
| Time | S. aureus | E. coli |

| (sec) | 10⁵ CFU | 10⁶ CFU | 10⁵ CFU | 10⁶ CFU |
|---|---|---|---|---|
| 0 | 438 | 1167 | 533 | 1169 |
| 10 | 2381 | 8279 | 22776 | 13951 |
| 20 | 2677 | 8273 | 26139 | 16023 |
| 30 | 3216 | 11174 | 26044 | 18415 |
| 40 | 4049 | 14556 | 25732 | 23670 |
| 50 | 4999 | 18989 | 25341 | 27481 |
| 60 | 6098 | 25040 | 24953 | 30280 |
| 70 | 7078 | 33423 | 24659 | 33077 |
| 80 | 8034 | 52090 | 24236 | 35107 |
| 90 | 8896 | 68418 | 23803 | 37464 |
| 100 | 9694 | 74989 | 23569 | 40172 |
| 110 | 10412 | 84991 | 23203 | 42787 |
| 120 | 10951 | 92328 | 22786 | 45949 |
| 130 | 11458 | 105210 | 22422 | 54125 |
| 140 | 11984 | 108477 | 22265 | 68429 |
| 150 | 12440 | 118505 | 21981 | 76109 |
| 160 | 12771 | 124434 | 21639 | 85564 |
| 170 | 13184 | 136390 | 21339 | 101311 |
| 180 | 13655 | 141752 | 21122 | 112249 |
| 190 | 13948 | 145560 | 20828 | 143322 |
| 200 | 14372 | 148799 | 20517 | 159694 |
| 210 | 14740 | 152368 | 20395 | 173869 |
| 220 | 15273 | 155312 | 20118 | 190660 |
| 230 | 15785 | 158528 | 19992 | 201130 |
| 240 | 16178 | 161061 | 19649 | 211916 |

About 5 mg of BARDAC 205M fibers were added to the tubes immediately after the T₀ measurement was recorded.
All measurements are reported in relative light units (RLU's).

### Example 21

### Detection of ATP from Suspensions of Live Microbial Cells Exposed to Aqueous Extractant

BARDAC 205M was diluted in water to achieve 0.1%, 0.5%, and 1% solution in water. S. aureus and E. coli overnight culture was prepared as described in Example 1 and the cells were diluted in Butterfield's buffer. One hundred microliters of luciferin/luciferase reagent from Clean-Trace surface ATP system was added to 1.5 ml microfuge tubes containing approximately 10⁵ cells. Immediately after adding the bacterial suspension, the tube was placed into a bench-top luminometer (20/20n single tube luminometer with software), as described in Example 9, and an initial (T₀) measurement of RLUs was recorded. One to 5 microliters of BARDAC 205M solution was added to each of the microfuge tubes and the resulting luminescence was monitored in a bench top luminometer (20/20n single tube luminometer). The results are shown in Table 26. The effective concentration of BARDAC 205M to achieve good signal was between 0.0025 to 0.005%.

TABLE 26
Detection of ATP from cells exposed to a cell extractant in an aqueous solution. About 1 to 5 microliter of BARDAC
205M solution was added to the tubes immediately after the T₀ measurement was recorded.
All measurements are reported in relative light units (RLU's).

| Time | SA 6538 10⁵ CFU | | | | | | EC 51183 10⁵ CFU | | | | | |
|---|---|---|---|---|---|---|---|---|---|---|---|---|
| (sec) | 0.0005% | 0.001% | 0.0025% | 0.005% | 0.010% | 0.025% | 0.0005% | 0.001% | 0.0025% | 0.005% | 0.010% | 0.025% |
| 0 | 1061 | 1838 | 1865 | 1004 | 1955 | 1715 | 683 | 780 | 865 | 985 | 955 | 351 |
| 10 | 1664 | 3423 | 9589 | 63723 | 31953 | 6330 | 1778 | 3667 | 9463 | 43499 | 44723 | 347 |
| 20 | 1854 | 3594 | 15966 | 78217 | 43709 | 2533 | 1910 | 3864 | 11324 | 52764 | 46923 | 345 |
| 30 | 2361 | 3883 | 22870 | 80657 | 46535 | 1147 | 1990 | 4008 | 14362 | 53255 | 47778 | 323 |
| 40 | 3183 | 4222 | 28830 | 81722 | 47465 | 608 | 2116 | 4070 | 18241 | 53005 | 48015 | 319 |
| 50 | 4027 | 4484 | 38514 | 82869 | 47918 | 422 | 2164 | 4161 | 25951 | 52903 | 48099 | 321 |
| 60 | 4845 | 4781 | 42670 | 83720 | 47981 | 362 | 2244 | 4234 | 30264 | 52739 | 48339 | 324 |
| 90 | 7545 | 5324 | 53874 | 85074 | 47857 | 288 | 2761 | 4532 | 48528 | 51730 | 48074 | 293 |
| 120 | 9243 | 5881 | 61800 | 85793 | 47444 | 268 | 3668 | 5188 | 62269 | 50529 | 47462 | 283 |
| 150 | 11310 | 6426 | 65541 | 85879 | 46911 | 246 | 4711 | 6340 | 62410 | 49477 | 46867 | 281 |
| 180 | 12450 | 6798 | 66409 | 86130 | 46364 | 235 | 5523 | 7518 | 61184 | 48891 | 46615 | 261 |
| 210 | 14732 | 7383 | 66799 | 86055 | 45803 | 222 | 6957 | 10025 | 59894 | 48064 | 45890 | 253 |
| 240 | 16539 | 8024 | 66567 | 86118 | 44948 | 202 | 8577 | 13338 | 58471 | 47184 | 45411 | 226 |

### Example 22

### Luciferin Hydrogel Beads

Hydrogel beads containing luciferin were made either using direct method (Preparative Example 3) or by post-absorption (Preparative Example 7).

Microfuge tubes were set up containing 100 μl of PBS, 10 μl of 1 μM ATP and 1 μl of 6.8 μg/ml luciferase. Background reading was taken in a bench top luminometer (20/20n single tube luminometer with software), as described in Example 9, and hydrogel beads containing luciferin were added to the tube and reading was followed at 10 sec interval. The post-absorbed beads were more active than the preparative beads (Table 27).

TABLE 27
ATP bioluminescence using luciferin hydrogel beads.

| Time (sec) | Luciferin-1s bead | Luciferin-1p bead |
|---|---|---|
| 0 | 135 | 114 |
| 10 | 33587 | 366562 |
| 20 | 32895 | 365667 |
| 30 | 32297 | 360779 |
| 40 | 31914 | 356761 |
| 50 | 31721 | 353358 |
| 60 | 31524 | 348912 |

Luciferin bead was added to the sample immediately after the T₀ measurement was obtained.

### Example 23

### Luciferase Hydrogel Beads

Hydrogel beads containing luciferase were made either using direct method (Preparative Example 4) or by post-absorption (Preparative Example 8).

Microfuge tubes were set up containing 100 microliter of luciferase assay substrate buffer (Promega Corporation, Madison, Wis.) Background reading was taken in a bench top luminometer (20/20n single tube luminometer with software, as described in Example 9) and hydrogel beads containing luciferase were added to the tube and reading was followed at 10 sec interval. Both types of beads showed good activity (Table 28).

TABLE 28
ATP bioluminescence using luciferase hydrogel beads.

| Time (sec) | Luciferase-1s bead | Luciferase-1p bead |
|---|---|---|
| 0 | 85 | 112 |
| 10 | 2757674 | 2564219 |
| 20 | 4790253 | 2342682 |
| 30 | 7079855 | 2201900 |
| 40 | 12865862 | 2142650 |
| 50 | 16588018 | 2048034 |
| 60 | 21054562 | 1958730 |
| 70 | 26456702 | 1886521 |

Luciferase bead was added to the sample immediately after the T₀ measurement was obtained.

In a similar experiment, effect of increasing number of post-absorbed luciferase beads was tested. Microfuge tubes containing 100 microliter of luciferase assay substrate buffer (Promega) were set up and luciferase hydrogel beads (1-4 beads per tube) were added. The luminescence was monitored immediately in a bench top luminometer (FB-12 single tube luminometer with software as described in Example 2). The experiment was done in triplicates. The results, shown in Table 29, indicate a generally linear relationship between the number of beads per tube and the amount of luciferase activity.

TABLE 29
Detection of luciferase activity in hydrogel beads.

| | 0 beads | 1 bead | 2 beads | 3 beads | 4 beads |
|---|---|---|---|---|---|
| Trial 1 | 1379 | 2148034 | 3302458 | 4734298 | 5130662 |
| Trial 2 | 609 | 1858030 | 2975657 | 4364022 | 5090202 |
| Trial 3 | 602 | 1788521 | 2806418 | 4144277 | 4831947 |
| Average | 863 | 1931528 | 3028178 | 4414199 | 5017604 |

Luciferase-1p beads containing luciferase enzyme were added to the tubes containing luciferase assay buffer and measurements were obtained.
All measurements are reported in relative light units (RLU's).

### Example 24

### Effect of Cell Extractant-Loaded Microtablets on the Release of ATP from S. aureus and E. coli

Cell extractant-loaded microtablets were prepared as described in Preparative Example 9. S. aureus ATCC 6538 and E. coli ATCC 51183 were obtained from ATCC (Manassas, Va.). 3M™ Clean-Trace™ Surface ATP system and NG Luminometer UNG2 were obtained from 3M Company (St. Paul, Minn.). Pure cultures of the bacterial strains were inoculated into tryptic soy broth and were grown overnight at 37° C. The luciferase/luciferin liquid reagent solution (100 μl) was removed from Clean-Trace surface ATP hygiene test units and transferred to 1.5 ml microfuge tubes. The bacterial culture was diluted to approximately 10⁷ CFU/ml in Butterfield's buffer and 100 microliters of the diluted suspension were added directly to individual microfuge tubes (i.e., approximately 10⁶ CFU per tube). Immediately after adding the bacterial suspension, the tube was placed into a bench-top luminometer (20/20n single tube luminometer, Turner Biosystems, Sunnyvale, Calif.) and an initial (T₀) measurement of the relative light units (RLUs) was recorded. The initial (and all subsequent luminescence measurements) were obtained from the luminometer using 20/20n SIS software that was provided with the luminometer. The light signal was integrated for 1 second and the results are expressed in RLU/sec.

After taking T₀ measurement, a microtablet containing a cell extractant was added to individual tubes and RLU measurements were recorded at 10 sec intervals until the number of RLUs reached a plateau (Tables 30 and 31). The microtablets containing cell extractants, extracted ATP from the S. aureus and E. coli and the ATP reacted with the ATP-detection reagents to elicit bioluminescence. The RLU increased over time in the tubes that received the extractant-loaded microtablets, while the tubes with control microtablets did not show a significant increase in RLU over time.

TABLE 30
Detection of ATP released from S. aureus cells (10⁶ cfu) after exposure to extractant-loaded microtablet.
Values expressed in the table are relative light units (RLUs).
Microtablets were added to the sample immediately after the T₀ measurement was obtained.

| | | 0.025% | 0.05% | 0.025% | 0.05% | | | | |
|---|---|---|---|---|---|---|---|---|---|
| Time | | Benzalkonium | benzalkonium | CHG | CHG | 0.025% | 0.05% | 0.025% CHG | 0.05% CHG |
| (sec) | Control | chloride | chloride | diacetate | diacetate | CTAB | CTAB | dihydrochloride | dihydrochloride |
| 0 | 2302 | 2145 | 2992 | 2333 | 2206 | 2836 | 2803 | 2681 | 2996 |
| 10 | 3208 | 58360 | 6827 | 6609 | 8624 | 9273 | 7602 | 49220 | 8095 |
| 20 | 4192 | 120557 | 14320 | 7922 | 13240 | 14728 | 11728 | 49513 | 9649 |
| 30 | 5153 | 134708 | 22609 | 9468 | 18915 | 21521 | 18646 | 49360 | 11690 |
| 40 | 5797 | 142357 | 31837 | 11007 | 25334 | 29616 | 26815 | 49655 | 13764 |
| 50 | 5742 | 147199 | 41950 | 12607 | 32168 | 38379 | 34375 | 50029 | 15886 |
| 60 | 5718 | 151117 | 52219 | 14333 | 38462 | 47791 | 38733 | 50372 | 18101 |
| 90 | 5668 | 159451 | 81746 | 20375 | 53812 | 90012 | 28143 | 51017 | 24547 |
| 120 | 5563 | 163527 | 102420 | 28118 | 65078 | 107772 | 16482 | 51810 | 31124 |
| 150 | 5462 | 167094 | 121184 | 41756 | 73732 | 113692 | 9466 | 52648 | 38821 |
| 180 | 5424 | 169073 | 128600 | 54355 | 83217 | 113419 | 6195 | 53693 | 47340 |
| 210 | 5385 | 171166 | 132192 | 66778 | 89249 | 111501 | 4430 | 54717 | 56551 |
| 240 | 5332 | 172314 | 135585 | 76906 | 94657 | 109286 | 3303 | 55899 | 66612 |
| 270 | 5257 | 173148 | 137086 | 85446 | 100242 | 107535 | 2577 | 56831 | 76267 |
| 300 | 5221 | 173786 | 138250 | 93022 | 107421 | 105793 | 2056 | 57798 | 85881 |
| 330 | 5197 | 174002 | 139081 | 99662 | 112682 | 104114 | 1726 | 58507 | 95772 |
| 360 | 5107 | 174300 | 139278 | 105602 | 119313 | 102119 | 1446 | 59266 | 105455 |

TABLE 31
Detection of ATP released from E. coli cells (10⁶ cfu) after exposure to extractant-loaded microtablet.
Values expressed in the table are relative light units (RLUs).
Microtablets were added to the sample immediately after the T₀ measurement was obtained.

| | | 0.025% | 0.05% | 0.025% | 0.05% | | | | |
|---|---|---|---|---|---|---|---|---|---|
| Time | | Benzalkonium | benzalkonium | CHG | CHG | 0.025% | 0.05% | 0.025% CHG | 0.05% CHG |
| (sec) | Control | chloride | chloride | diacetate | diacetate | CTAB | CTAB | dihydrochloride | dihydrochloride |
| 0 | 3306 | 3769 | 3496 | 3051 | 3234 | 3738 | 3459 | 3524 | 3419 |
| 10 | 4374 | 5349 | 7897 | 8129 | 21045 | 5651 | 6046 | 9557 | 18146 |
| 20 | 4947 | 7446 | 8187 | 12163 | 29288 | 5873 | 8650 | 13507 | 34814 |
| 30 | 5272 | 8418 | 8624 | 17088 | 39518 | 6051 | 18491 | 18721 | 52153 |
| 40 | 5283 | 9412 | 9716 | 21900 | 49580 | 6264 | 35172 | 24608 | 68275 |
| 50 | 5216 | 11453 | 12367 | 26365 | 58158 | 6546 | 52404 | 30820 | 82285 |
| 60 | 5211 | 15426 | 17098 | 30359 | 65487 | 6961 | 55549 | 37310 | 94083 |
| 90 | 5174 | 29462 | 34295 | 39178 | 80896 | 9708 | 59149 | 56255 | 120491 |
| 120 | 5048 | 45443 | 43522 | 45151 | 90847 | 16102 | 60198 | 72270 | 134727 |
| 150 | 4983 | 60626 | 45011 | 49431 | 96726 | 25494 | 60458 | 84317 | 141245 |
| 180 | 4940 | 62090 | 45233 | 52166 | 99766 | 34815 | 60720 | 92892 | 143950 |
| 210 | 4942 | 62468 | 45195 | 53847 | 101186 | 42080 | 60511 | 99304 | 144682 |
| 240 | 4859 | 62570 | 44691 | 54921 | 101333 | 47364 | 60333 | 103261 | 144474 |
| 270 | 4790 | 62439 | 44503 | 55346 | 101205 | 50942 | 60349 | 105683 | 143755 |
| 300 | 4697 | 62188 | 44006 | 55675 | 100840 | 53777 | 60199 | 107040 | 142961 |
| 330 | 4706 | 62128 | 43847 | 55579 | 100259 | 55668 | 60159 | 108470 | 141678 |
| 360 | 4682 | 61760 | 42032 | 55682 | 99524 | 57214 | 59652 | 109255 | 140753 |

### Example 25

### Effect of Cell Extractant-Loaded Microtablets on the Release of ATP from S. aureus and E. coli

Cell extractant-loaded microtablets were prepared as described in Preparative Example 9. 3M™ Clean-Trace™ Surface ATP system and NG Luminometer UNG2 were obtained from 3M Company (St. Paul, Minn.). Polyester-tipped applicators (ATP controlled) were obtained from Puritan Medical Products (Guilford, Me.).

S. aureus overnight cultures were prepared as described in Example 2. Swabs from the Clean-Trace surface ATP hygiene tests, which include microbial cell extractants, were replaced with polyester-tipped applicators, which do not include microbial cell extractants. The bacterial culture was diluted to 10⁷ CFU/ml in Butterfield's buffer and 100 microliters of the diluted suspension were added directly to the swabs (i.e., approximately 10⁶ CFU per swab). One microtablet containing cell extractant was added to the test units and the swab was activated by pushing it into the reagent chamber according to the manufacturer's instructions. The test unit was immediately inserted into the reading chamber of a NG Luminometer, UNG2 and RLU measurements were recorded at 10 sec interval using Unplanned Testing mode until the number of RLUs reached a plateau. The data was downloaded using the software provided with the NG luminometer. The microtablets containing cell extractants were able to lyse bacteria and release ATP from cells. The relative light units (RLU) increased over time in the tubes that received the extractant-loaded microtablets, while the tubes with control microtablets did not show a significant increase in RLU over time (Table 32)

TABLE 32
Detection of ATP from S. aureus cells (circa 10⁶ cfu) after exposure to
extractant-loaded microtablet. Values expressed in the table are relative light units
(RLUs). Microtablet containing extractants or control microtablet was added to the
sample and readings were taken at defined intervals.

| | | 0.025% | 0.05% | 0.025% | | |
|---|---|---|---|---|---|---|
| Time | | Benzalkonium | benzalkonium | CHG | 0.025% | 0.025% CHG |
| (sec) | Control | chloride | chloride | diacetate | CTAB | dihydrochloride |
| 10 | 117 | 773 | 434 | 788 | 322 | 1726 |
| 20 | 114 | 912 | 463 | 848 | 416 | 1939 |
| 30 | 120 | 1039 | 489 | 975 | 524 | 2311 |
| 40 | 128 | 1167 | 533 | 1119 | 582 | 3181 |
| 50 | 129 | 1313 | 592 | 1228 | 599 | 3645 |
| 60 | 127 | 1461 | 4678 | 1333 | 610 | 4099 |
| 90 | 134 | 2109 | 4737 | 1546 | 618 | 5318 |
| 120 | 145 | 3447 | 4644 | 1738 | 612 | 6056 |
| 150 | 155 | 4937 | 4751 | 1925 | 616 | 6486 |
| 180 | 157 | 6980 | 5342 | 9270 | 624 | 16563 |
| 210 | 165 | 9166 | 5433 | 15747 | 2371 | 27414 |
| 240 | 168 | 10481 | 5407 | 20262 | 18471 | 29035 |
| 270 | 172 | 11561 | 5417 | 22166 | 22864 | 29156 |
| 300 | 171 | 11930 | 5411 | 24080 | 23182 | 27773 |

### Example 26

### ATP Bioluminescence Using Luciferin-Luciferase Microtablets

Microtablets containing luciferase and luciferin were prepared as described in Preparative Example 10. Microfuge tubes were set up containing 190 μl of Butterfield's buffer. Ten microliters of 1 μM ATP (Sigma-Aldrich) solution in sterile water was added to the tube. The microtablets containing luciferase and luciferin were added to the tube and the tube was placed into a bench-top luminometer (20/20n single tube luminometer). Measurement of RLUs was recorded at 10 sec interval using 20/20n SIS software. The light signal was integrated for 1 second and the results are expressed in RLU/sec. The bioluminescence (RLU) increased with addition of microtablets while without microtablets the back ground did not increase. ATP bioluminescence was also measured using the formulation used for lyophilization. ATP bioluminescence gradually increased in tubes with enzyme microtablets, while the relative light units peaked with in 10 to 20 sec with liquid formulation (Table 32 and Table 33).

TABLE 33
Detection of ATP bioluminescence from 10 picomoles of ATP after
exposure to luciferin-UltraGlo luciferase loaded microtablet.

| | | UltraGlo- | | |
|---|---|---|---|---|
| Time | UltraGlo-Luciferin | Luciferin | UltraGlo | UltraGlo |
| (sec) | microtablet_3 mg | microtablet_6 mg | formulation_20 μl | formulation_40 μl |
| 10 | 12005 | 30627 | 164874 | 389466 |
| 20 | 23626 | 80702 | 176134 | 431573 |
| 30 | 35128 | 125164 | 177319 | 441088 |
| 40 | 44406 | 162179 | 176850 | 442968 |
| 50 | 51503 | 194618 | 176254 | 441859 |
| 60 | 56965 | 226842 | 175169 | 440080 |
| 90 | 78549 | 312400 | 172680 | 432370 |
| 120 | 94078 | 363253 | 170322 | 423435 |
| 150 | 111429 | 419982 | 167591 | 414718 |
| 180 | 123502 | 455129 | 165407 | 406245 |
| 210 | 125966 | 489231 | 162992 | 397613 |
| 240 | 126877 | 489512 | 160530 | 389522 |

Values expressed in the table are relative light units (RLUs).
Microtablet containing luciferin-luciferase was added to the sample and readings were taken at defined intervals.

TABLE 34
Detection of ATP bioluminescence from 10 picomoles of
ATP after exposure to luciferin-luciferase loaded microtablet.

| | Luciferase- | Luciferase- | | |
|---|---|---|---|---|
| Time | luciferin | luciferin | Luciferase | Luciferase |
| (sec) | microtablet_2 mg | microtablet_4 mg | formulation_40 μl | formulation_60 μl |
| 10 | 1521 | 1888 | 36788 | 93019 |
| 20 | 3360 | 4727 | 37001 | 92668 |
| 30 | 5371 | 8894 | 37018 | 92410 |
| 40 | 7681 | 14991 | 37033 | 91830 |
| 50 | 10335 | 22650 | 37036 | 91144 |
| 60 | 13247 | 30920 | 37108 | 90621 |
| 90 | 21744 | 46708 | 36945 | 89408 |
| 120 | 31571 | 59101 | 36939 | 88201 |
| 150 | 39176 | 63330 | 36854 | 86833 |
| 180 | 40622 | 62910 | 36793 | 85631 |
| 210 | 41255 | 61634 | 36753 | 84791 |
| 240 | 40855 | 59926 | 36662 | 83584 |

Values expressed in the table are relative light units (RLUs).
Microtablet containing luciferin-luciferase was added to the sample and readings were taken at defined intervals.

### Example 27

### Effect of Cell Extractant-Loaded Films on the Release of ATP from S. aureus and E. coli

S. aureus ATCC 6538 and E. coli ATCC 51183 were obtained from the American Type Culture Collection (Manassas, Va.). 3M™ Clean-Trace™ Surface ATP system swabs were obtained from 3M Company (St. Paul, Minn.). A bench top luminometer (20/20n single tube luminometer) with 20/20n SIS software was obtained from Turner Biosystems (Sunnyvale, Calif.). Cell extractant-loaded disks and negative control disks were prepared as described in Preparative Example 11.

Reagent (300 μl) from Clean Trace ATP system was removed and added to 1.5 ml microfuge tube. Pure cultures of the bacterial strains were inoculated into tryptic soy broth and were grown overnight at 37° C. The bacteria were diluted in Butterfield's diluent to obtain suspensions containing approximately 10⁷ and 10⁸ colony-forming units (CFU) per milliliter, respectively. Ten microliter aliquots of the diluted suspensions were added to separate microfuge tubes containing the ATP detection reagent, thereby resulting in tubes containing approximately 10⁵ and 10⁶ CFU, respectively.

Immediately after adding the bacterial suspension, the microfuge tube was placed into the luminometer and an initial (Tₒ) measurement of RLUs was recorded. The initial measurement and all subsequent luminescence measurements were obtained from the luminometer using the 20/20n SIS software. The light signal was integrated for 1 second and all results are expressed in RLU/sec.

After taking T₀ measurement, a disk containing cell extractant was added to the tube and RLU measurements were recorded at 10 sec intervals until the number of RLUs reached a plateau (Table 35). The film containing cell extractants cause the release of ATP from the S. aureus and E. coli and the ATP reacted with the ATP-detection reagents to elicit bioluminescence. Tubes receiving the extractant-loaded films showed a relatively large increase in RLU during the observation period. The magnitude of the increase was related to the number of bacteria inoculated into the tube. In contrast, tubes receiving the negative control film showed a relatively small increase in RLU during the observation period.

TABLE 35
Detection of ATP from microbial cells exposed to microbial cell
extractants released from coated films. Values expressed in the table
are relative light units (RLUs). A single of control or coated films
(~7 mm) was added to the sample immediately after the
T₀ measurement was obtained.

| | S. aureus | | | E. coli | | |
|---|---|---|---|---|---|---|
| | Control | VANTOCIL/ | | Control | VANTOCIL/ | |
| Time | film | CARBOSHIELD film | | film | CARBOSHIELD film | |
| (sec) | 10⁶ cfu | 10⁵ cfu | 10⁶ cfu | 10⁶ cfu | 10⁵ cfu | 10⁶ cfu |
| 0 | 1307 | 504 | 1371 | 1115 | 571 | 1036 |
| 10 | 3126 | 4966 | 4590 | 2283 | 2123 | 3643 |
| 20 | 3096 | 5468 | 4856 | 2324 | 2093 | 3624 |
| 30 | 3253 | 8166 | 5852 | 2620 | 2238 | 3785 |
| 40 | 3317 | 11018 | 9235 | 2754 | 2513 | 3830 |
| 50 | 3256 | 16987 | 15716 | 2805 | 2806 | 4055 |
| 60 | 3397 | 19263 | 26021 | 2684 | 2987 | 4201 |
| 90 | 3470 | 26137 | 59849 | 2910 | 3846 | 5480 |
| 120 | 3562 | 32194 | 92486 | 3009 | 5449 | 7869 |
| 150 | 3675 | 35334 | 118780 | 3042 | 7497 | 12877 |
| 180 | 3702 | 38054 | 144637 | 3065 | 10387 | 17589 |
| 210 | 3838 | 41171 | 168549 | 3326 | 11867 | 21509 |
| 240 | 3840 | 43361 | 187939 | 3294 | 12410 | 24923 |
| 270 | 3954 | 45679 | 204903 | 3237 | 12964 | 27259 |
| 300 | 3948 | 47729 | 220871 | 3225 | 12965 | 29268 |
| 330 | 3922 | 49905 | 234051 | 3102 | 12982 | 31175 |
| 360 | 3958 | 51834 | 246106 | 3135 | 12909 | 32592 |

### Example 28

### Effect of Cell Extractant-Loaded Matrices on the Release of ATP from S. aureus and E. coli

Cell extractant-loaded matrices were prepared as described in Preparative Example 12. S. aureus and E. coli overnight cultures were prepared as described in Example 2. Reagent (600 μl) from Clean Trace ATP system was removed and added to 1.5 ml microfuge tube. About 10⁶ cfu of bacteria in Butterfield's buffer (10 μl) were added directly to the microfuge tube. Immediately after adding the bacterial suspension, a disk (ca. 7 mm) of various matrices containing cell extractant was added to the tube. The tube was immediately placed into a bench-top luminometer (20/20n single tube luminometer) and RLU measurements were recorded at 10 sec intervals until the number of RLUs reached a plateau (Tables 36-39). All luminescence measurements were obtained from the luminometer using 20/20n SIS software. The light signal was integrated for 1 second and the results are expressed in RLU/sec.

All the matrices, except grade 54 filter paper and grade 30 glass-fiber filter paper, coated with VANTOCIL-CARBOSHIELD solution with a binder showed a gradual release of ATP as the RLU increased over time. In case of matrices coated with 5% benzalkonium chloride, only few matrices (grade 4, grade 54 and GB005 blotting paper) showed some gradual release of ATP.

TABLE 36
Detection of ATP from S. aureus cells (approximately 10⁶ cfu) exposed to
5% benzalkonium chloride released from various matrices.
Values expressed in the table are relative light units (RLUs).
A single disk of the matrix (circa 7 mm diameter)
was added to the sample and readings were obtained at defined intervals.

| | | Grade | | | | |
|---|---|---|---|---|---|---|
| | Grade 4 | 54 | Grade 30 | MF- | GB005 | Zeta Plus |
| Time | Filter | Filter | Glass-Fiber | Millipore | Blotting | Virosorb |
| (sec) | paper | paper | Filter paper | membrane | paper | 1MDS |
| 10 | 31466 | 8271 | 2064 | 17446 | 19450 | 1619 |
| 20 | 62365 | 13536 | 883 | 2199 | 32138 | 1677 |
| 30 | 81948 | 17652 | 658 | 926 | 37630 | 1809 |
| 40 | 96812 | 21624 | 548 | 673 | 39797 | 1996 |
| 50 | 108944 | 25571 | 487 | 595 | 40721 | 2165 |
| 60 | 119634 | 29431 | 437 | 575 | 41498 | 2341 |
| 90 | 152643 | 40449 | 374 | 530 | 44448 | 3003 |
| 120 | 179784 | 51917 | 338 | 468 | 47270 | 3909 |
| 150 | 189754 | 62779 | 298 | 440 | 50865 | 4808 |
| 180 | 182587 | 72922 | 271 | 416 | 54810 | 5912 |
| 210 | 181958 | 82731 | 255 | 403 | 58285 | 7000 |
| 240 | 187022 | 93057 | 236 | 390 | 61388 | 8209 |
| 270 | 187087 | 103203 | 221 | 370 | 64577 | 9411 |
| 300 | 186538 | 113069 | 213 | 351 | 68662 | 10470 |
| 330 | 187524 | 122326 | 191 | 353 | 72843 | 11554 |
| 360 | 185621 | 130957 | 177 | 353 | 77992 | 12501 |

TABLE 37
Detection of ATP from E. coli cells (approximately 10⁶ cfu) exposed to
5% benzalkonium chloride released from various matrices. Values
expressed in the table are relative light units (RLUs). A single disk of
the matrix (circa 7 mm diameter) was added to the sample and readings
were obtained at defined intervals.

| | | Grade | | | | |
|---|---|---|---|---|---|---|
| | Grade 4 | 54 | Grade 30 | MF- | GB005 | Zeta Plus |
| Time | Filter | Filter | Glass-Fiber | Millipore | Blotting | Virosorb |
| (sec) | paper | paper | Filter paper | membrane | paper | 1MDS |
| 10 | 3625 | 2662 | 4937 | 4020 | 11249 | 1210 |
| 20 | 4907 | 3170 | 667 | 5694 | 18274 | 1567 |
| 30 | 6010 | 4043 | 323 | 561 | 21017 | 1812 |
| 40 | 6800 | 5067 | 258 | 795 | 21276 | 1999 |
| 50 | 7610 | 5886 | 212 | 537 | 20460 | 2176 |
| 60 | 8197 | 6752 | 206 | 456 | 19435 | 2378 |
| 90 | 9164 | 8756 | 180 | 121 | 16832 | 3013 |
| 120 | 9248 | 11323 | 158 | 117 | 14796 | 3998 |
| 150 | 9395 | 13223 | 146 | 112 | 12938 | 4956 |
| 180 | 9650 | 15189 | 137 | 103 | 11157 | 5989 |
| 210 | 9895 | 16945 | 133 | 102 | 9492 | 7045 |
| 240 | 10223 | 18641 | 124 | NR | 8020 | 8267 |
| 270 | 10558 | 20398 | 122 | NR | 6796 | 9456 |
| 300 | 10863 | 22129 | 118 | NR | 5788 | 10345 |
| 330 | 11215 | 23812 | 115 | NR | NR | 11545 |
| 360 | 11615 | 25398 | 120 | NR | NR | 12456 |

NR = not recorded

TABLE 38
Detection of ATP from S. aureus cells (approximately 10⁶ cfu) exposed to
VANTOCIL-CARBOSHIELD mixture released from various matrices.
Values expressed in the table are relative light units (RLUs).
A single disk of the matrix (circa 7 mm diameter) was added to the
sample and readings were obtained at defined intervals.

| | | Grade | | | | |
|---|---|---|---|---|---|---|
| | Grade 4 | 54 | Grade 30 | MF- | GB005 | Zeta Plus |
| Time | Filter | Filter | Glass-Fiber | Millipore | Blotting | Virosorb |
| (sec) | paper | paper | Filter paper | membrane | paper | 1MDS |
| 10 | 12533 | 122215 | 162572 | 22783 | 10089 | 2211 |
| 20 | 23884 | 166427 | 157524 | 44677 | 13877 | 3386 |
| 30 | 33479 | 186833 | 153505 | 61466 | 17157 | 4691 |
| 40 | 43074 | 192538 | 150667 | 72716 | 19794 | 6025 |
| 50 | 52869 | 196746 | 148459 | 80181 | 22125 | 7791 |
| 60 | 61801 | 196665 | 147092 | 86114 | 24406 | 9932 |
| 90 | 83487 | 193554 | 141504 | 99206 | 31155 | 21740 |
| 120 | 100308 | 189883 | 136611 | 108051 | 38947 | 34102 |
| 150 | 115974 | 185320 | 131514 | 113662 | 47406 | 43757 |
| 180 | 130275 | 180843 | 126167 | 117474 | 57022 | 51508 |
| 210 | 142159 | 177298 | 121535 | 119146 | 66161 | 57963 |
| 240 | 151160 | 173070 | 116543 | 120224 | 75526 | 62805 |
| 270 | 158786 | 168729 | 111283 | 120061 | 84389 | 67113 |
| 300 | 164282 | 164768 | 106385 | 120028 | 92369 | 70505 |
| 330 | 168307 | 160618 | 102496 | 119186 | 99493 | 73547 |
| 360 | 171095 | 156418 | 99095 | 118766 | 105782 | 75573 |

TABLE 39
Detection of ATP from E. coli cells (approximately 10⁶ cfu) exposed to
VANTOCIL-CARBOSHIELD mixture released from various matrices.
Values expressed in the table are relative light units (RLUs).
A single disk of the matrix (circa 7 mm diameter) was added to the
sample immediately and readings were obtained at defined intervals.

| | | Grade | | | | |
|---|---|---|---|---|---|---|
| | Grade 4 | 54 | Grade 30 | MF- | GB005 | Zeta Plus |
| Time | Filter | Filter | Glass-Fiber | Millipore | Blotting | Virosorb |
| (sec) | paper | paper | Filter paper | membrane | paper | 1MDS |
| 10 | 19039 | 201835 | 58807 | 12473 | 19479 | 2596 |
| 20 | 24026 | 214879 | 86175 | 15083 | 17871 | 4578 |
| 30 | 30338 | 213890 | 100026 | 18254 | 18198 | 8567 |
| 40 | 36529 | 212007 | 108645 | 21372 | 19214 | 14154 |
| 50 | 42453 | 210568 | 116805 | 24365 | 20563 | 20359 |
| 60 | 47578 | 208763 | 123915 | 27176 | 22210 | 26566 |
| 90 | 60095 | 202697 | 139268 | 35702 | 27659 | 40841 |
| 120 | 69576 | 197701 | 146390 | 43155 | 34776 | 51014 |
| 150 | 76577 | 192634 | 160193 | 49634 | 42290 | 58057 |
| 180 | 81739 | 186981 | 174138 | 55092 | 49748 | 63562 |
| 210 | 85113 | 181744 | 186111 | 59506 | 57996 | 67315 |
| 240 | 86920 | 176901 | 194237 | 63133 | 66248 | 70338 |
| 270 | 88241 | 171903 | 199627 | 66202 | 75518 | 72479 |
| 300 | 88692 | 166875 | 201583 | 68956 | 84828 | 74107 |
| 330 | 88609 | 162292 | 203241 | 71460 | 93669 | 75174 |
| 360 | 87900 | 157370 | 203570 | 73508 | 100857 | 75421 |

### Example 29

### Effect of Chlorohexidine Gluconate (CHG) Gel on the Release of ATP from S. aureus and E. coli

S. aureus and E. coli overnight cultures were prepared as described in Example 2. CHG Tegaderm containing a gel with 2% CHG was obtained from 3M, St. Paul. Reagent (600 μl) from Clean Trace ATP system was removed and added to 1.5 ml microfuge tube. About 10⁶ cfu of bacteria in Butterfield's buffer (10 μl) were added directly to the microfuge tube. Immediately after adding the bacterial suspension, a known amount of CHG gel was added to the tube. The tube was immediately placed into a bench-top luminometer (20/20n single tube luminometer) and RLU measurements were recorded at 10 sec intervals until the number of RLUs reached a plateau (Table 40). All luminescence measurements were obtained from the luminometer using 20/20n SIS software. The light signal was integrated for 1 second and the results are expressed in RLU/sec.

The CHG gel extracted ATP from the S. aureus and E. coli and the ATP reacted with the ATP-detection reagents to elicit bioluminescence. The RLU increased over time indicating release of ATP from cells.

TABLE 40
Detection of ATP from microbial cells exposed to CHG gel.

| | |
|---|---|
| S. aureus (~10⁶ cfu) | E. coli (~10⁶ cfu) |

| Time (sec) | 80 mg | 220 mg | 80 mg | 220 mg |
|---|---|---|---|---|
| 10 | 7032 | 7217 | 12983 | 14405 |
| 20 | 8026 | 7975 | 13538 | 15851 |
| 30 | 8864 | 8775 | 14060 | 17227 |
| 40 | 9703 | 9508 | 14490 | 18274 |
| 50 | 10806 | 10289 | 14840 | 19171 |
| 60 | 12003 | 11099 | 15153 | 20104 |
| 90 | 15391 | 14039 | 15923 | 23031 |
| 120 | 18840 | 17534 | 17047 | 26481 |
| 150 | 22200 | 21207 | 18173 | 30719 |
| 180 | 25352 | 25043 | 19427 | 35459 |
| 210 | 28213 | 28886 | 20803 | 40422 |
| 240 | 30582 | 32715 | 22077 | 45534 |
| 270 | 33018 | 36253 | 23241 | 50560 |
| 300 | 35178 | 39658 | 24532 | 55358 |
| 330 | 37187 | 42648 | 25729 | 60054 |
| 360 | 39290 | 45777 | 26965 | 64312 |

Values expressed in the table are relative light units (RLUs).
CHG gel was added to the samples and measurements were obtained at defined intervals.

### Example 30

### ATP Bioluminescence Assay

Microfuge tubes were set up containing 190 μl of Butterfield's buffer. Ten microliters of 1 μM ATP (Sigma-Aldrich) solution in sterile water was added to the tube. A solution containing luciferase (7.8 mg/lit, 3M Bridgend, UK) and luciferin (5.5 mg/lit, Promega) in 14 mM in Phosphate buffer was prepared. A known amount of the luciferin-luciferase solution was added to the tube and the tube was placed into a bench-top luminometer (20/20n single tube luminometer). Measurement of RLUs was recorded at 10 sec interval using 20/20n SIS software. The light signal was integrated for 1 second and the results are expressed in RLU/sec. The relative light units peaked with in 10 to 20 sec with liquid formulation (Table 41).

TABLE 41
Detection of ATP bioluminescence from 10 picomoles of ATP.

| Time | Luciferase | Luciferase |
|---|---|---|
| (sec) | formulation_40 μl | formulation_60 μl |
| 10 | 36788 | 93019 |
| 20 | 37001 | 92668 |
| 30 | 37018 | 92410 |
| 40 | 37033 | 91830 |
| 50 | 37036 | 91144 |
| 60 | 37108 | 90621 |
| 90 | 36945 | 89408 |
| 120 | 36939 | 88201 |
| 150 | 36854 | 86833 |
| 180 | 36793 | 85631 |
| 210 | 36753 | 84791 |
| 240 | 36662 | 83584 |

Values expressed in the table are relative light units (RLUs).
Luciferin-luciferase solution was added to the sample and readings were taken at defined intervals.

Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1. An article for detecting cells in a sample, the article comprising:
a housing with an opening configured to receive a sample acquisition device;
a sample acquisition device; and
a release element comprising a cell extractant.

2. The article of claim 1, wherein the release element comprises an encapsulating agent.

3. The article of claim 2, wherein the encapsulating agent comprises a matrix.

4. The article of claim 3, wherein the matrix is a pre-formed matrix.

5. The article of claim 4, wherein the pre-formed matrix comprises a hydrogel.

6. The article of claim 4, wherein the pre-formed matrix comprises a nonwoven material.

7. The article of claim 5, wherein the nonwoven material is formed from a material selected from a group consisting of cellulose, glass, polyester, polyalkylene, polystyrene, and derivatives or combinations of any of the foregoing.

8. The article of claim 3, wherein the matrix comprises an admixture of the cell extractant and an excipient.

9. The article of claim 8, wherein the matrix comprises a tablet.

10. The article of claim 9, wherein the tablet further comprises an outer coating.

11. The article of claim 2, wherein the encapsulating agent further comprises a binder.

12. The article of claim 1, wherein the release element is disposed in the housing.

13. The article of claim 1, wherein the release element is disposed on the sample acquisition device.

14. The article of claim 13, wherein the sample acquisition device comprises a hollow shaft and wherein the release element is disposed in the hollow shaft.

15. The article of claim 1, wherein the sample acquisition device further comprises a reagent chamber.

16. The article of claim 15, wherein the reagent chamber comprises a detection reagent.

17. The article of claim 1, wherein the cell extractant is selected from the group consisting of a quaternary amine, a biguanide, a nonionic surfactant, a cationic surfactant, a phenolic, a cytolytic peptide, and an enzyme.

18. The article of claim 1, where the cell extractant is a microbial cell extractant.

19. The article of claim 1, further comprising a somatic cell extractant.

20. The article of claim 1, wherein the housing further comprises a frangible barrier that forms a compartment in the housing.

21. The article of claim 20, wherein the compartment comprises a detection reagent.

22. The article of claim 16, wherein the detection reagent is selected from the group consisting of an enzyme, an enzyme substrate, an indicator dye, a stain, an antibody, and a polynucleotide.

23. The article of claim 16, wherein the detection reagent comprises a reagent for detecting ATP.

24. The article of claim 23, wherein the detection reagent comprises luciferase or luciferin.

25. The article of claim 16, wherein the detection reagent comprises a reagent for detecting adenylate kinase.

26. The article of claim 20, wherein the frangible barrier comprises the release element comprising the cell extractant.

27. The article of claim 20, wherein the compartment comprises the release element.

28. An article for detecting cells in a sample, the article comprising:
a housing with an opening configured to receive a sample;
a release element comprising a cell extractant; and
a delivery element comprising a detection reagent.

29. The article of claim 28, wherein the release element and the delivery element are disposed in the housing

30. A sample acquisition device with a release element comprising a cell extractant disposed thereon.

31. A kit comprising a housing with an opening configured to receive a sample, a release element comprising a cell extractant, and a detection system.

32. The kit of claim 31, further comprising a sample acquisition device, wherein the opening of the housing is configured to receive the sample acquisition device.

33. The kit of claim 31, further comprising a delivery element comprising a detection reagent.

34. The kit of claim 31, wherein the cell extractant is a microbial cell extractant.

35. The kit of claim 34, further comprising a somatic cell extractant.

36. A method of detecting cells in a sample, the method comprising:
providing a release element comprising a cell extractant, and a sample suspected of containing cells;
forming a liquid mixture comprising the sample and the release element; and
detecting an analyte in the liquid mixture.

37. A method of detecting cells in a sample, the method comprising:
providing,
a sample acquisition device;
a housing with an opening configured to receive the sample acquisition device, and a release element comprising a cell extractant disposed therein;
obtaining sample material with the sample acquisition device;
forming in the housing a liquid mixture comprising the sample material and the release element; and
detecting an analyte in the liquid mixture.

38. The method of claim 36, further comprising providing a detection system and wherein detecting an analyte comprises using the detection system.

39. The method of claim 36, wherein detecting an analyte comprises detecting an analyte associated with a microbial cell.

40. The method of claim 39, wherein detecting an analyte comprises detecting an enzyme released from a live cell in the sample.

41. The method of claim 36, further comprising the steps of providing a somatic cell extractant and contacting the sample with the somatic cell extractant.

42. The method of claim 36, wherein detecting an analyte comprises quantifying an amount of the analyte.

43. The method of claim 42, wherein the amount of the analyte is quantified two or more times.

44. The method of claim 43, wherein the amount of analyte detected at a first time point is compared to the amount of analyte detected at a second time point.

45. The method of claim 36, wherein detecting an analyte comprises detecting ATP from cells.

46. The method of claim 36, wherein detecting an analyte comprises detecting the analyte immunologically or genetically.

47. The method of claim 36, wherein detecting an analyte comprises detecting colorimetrically, fluorimetrically, or lumimetrically.

48. The method of claim 36, further comprising the step of releasing the cell extractant from the release element using a release factor.
